(19) United States
(12) Patent Application Publication
(10) Pub. No.: US 2026/0355262 A1
(43) Pub. Date: Nov. 12, 2026

(54) Probiotic Derived Compounds that Inhibit Biofilm Formation and Improve Antibiotic Sensitivity Against Staphylococcus Aureus and Uses Thereof

(71) Applicants: Marquette University, Milwaukee, WI (US); Microbial Discovery Group, Oak Creek, WI (US); Wisconsin Alumni Research Foundation, Madison, WI (US)

(72) Inventors: Kyle R. Leistikow, Cudahy, WI (US); Krassimira R. Hristova, Wauwatosa, WI (US); Cameron R. Currie, Burlington (CA); Daniel S. May, Chestertown, MD (US)

(21) Appl. No.: 19/488,539

(22) PCT Filed: May 30, 2024
(86) PCT No.: PCT/US2024/031793

§ 371 Date: Nov. 26, 2025

Related U.S. Application Data
(60) Provisional application No. 63/469,776, filed on May 30, 2023.
(60) Provisional application No. 63/537,148, filed on Sep. 7, 2023.

(51) Int. Cl. A61K 35/742; A01N 63/22; A01P 1/00; A61K 31/43; A61K 31/505; A61K 31/7036; A61K 38/12; A61P 31/04; C12N 1/20

(52) U.S. Cl. CPC A61K 35/742; A01N 63/22; A01P 1/00; A61K 31/43; A61K 31/505; A61K 31/7036; A61K 38/12; A61P 31/04; C12N 1/20

(57) ABSTRACT

Disclosed are novel extracts, compositions, and methods with Bacillus subtilis probiotic compounds for countering multidrug-resistant (MDR) pathogens, such as Staphylococcus aureus, including anti-biofilm, anti-bacterial, and anti-quorum sensing for MDR pathogens.

### CROSS-REFERENCE TO RELATED APPLICATIONS

This application claims the benefit of priority to U.S. Provisional Application No. 63/469,776, filed May 30, 2023, and U.S. Provisional Application No. 63/537,148, filed Sep. 7, 2023, each of which is incorporated by reference herein as if set forth in its entirety.

### GOVERNMENT RIGHTS

This invention was made with government support under grant number W9132T2220001 and W9132T2320003 awarded by the Department of Defense, Department of the Army, Office of Army Research, and grant number AI142720, awarded by the National Institutes of Health. The government has certain rights in this invention.

### BACKGROUND

Multidrug-resistant (MDR) bacterial infections are a top ten global public health threat, causing 2.8 million infections and 35,000 deaths annually in the US alone. In 2019, antimicrobial resistance accounted for an estimated 700,000 deaths worldwide, with 2.5 million infections in the United States that cost the economy more than $55 billion. According to the NIH, approximately 60% of all bacterial infections are caused by biofilm formation, an aggregated heterogenous community of cells that increases antibiotic resistance 1000-fold.

Staphylococcus aureus (S. aureus) is one of the most clinically important MDR pathogens in the world (cause of pneumonia, sepsis, endocarditis, and soft tissue infections) with infections leading to high rates of morbidity and mortality in both humans and animals. For S. aureus, 90% of all infections are attributed to biofilm formation. S. aureus's ability to form biofilm protects individual cells from antibiotics and promotes the transfer of antibiotic resistance genes. In addition to the obvious human health implications, even a 1% improvement in S. aureus infection costs would yield an annual cost savings of $5.8 million per year in the United States alone. There is a critical need to develop novel therapeutic and prophylactic strategies to inhibit biofilm growth and disassemble mature biofilm formation for MDR pathogens like S. aureus.

### SUMMARY

An aspect of the present disclosure are compositions containing a Bacillus subtilis 6D1 cell free extract (CFE). The compositions may further include an antibiotic. The Bacillus subtilis 6D1-CFE P1 is a 75% to 100% P1 glycoprotein cell-free fraction. In embodiments, the compositions are antimicrobial compositions. In embodiments, the compositions have anti-biofilm activity in a pathogenic bacterium.

A further aspect is a method of obtaining a B. subtilis 6D1 cell-free extract. The method includes preparing a cell-free extract from an isolated Bacillus subtilis culture and fractionating the cell-free extract with ethyl acetate to obtain fractionated extract with anti-biofilm activity. The method further includes separating the fractionated extract to obtain an active B. subtilis 6D1 CFE P1 glycoprotein cell free fraction. The method may further include purifying the B. subtilis 6D1 CFE.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

It should be understood that the drawings described below are for illustration purposes only and are not intended to limit the scope of the present disclosure in any way.

FIGS. 1A-IF demonstrate that Bacillus subtilis 6D1 cell free extract (CFE) inhibits S. aureus biofilm but not planktonic growth. Compared to 0.5× minimum inhibitory concentrations (MIC) of Sulfamethoxazole/Trimethoprim, addition of 10% v/v B. subtilis 6D1 CFE inhibits biofilm growth (FIG. 1B), but not planktonic growth (FIG. 1A). (***) P<0.001, (****) P<0.0001 compared to the untreated control. Competition experiments performed in planktonic and biofilm conditions for 24 hours show B. subtilis 6D1 outcompetes S. aureus in a biofilm but not in a planktonic environment (FIG. 1C), (**) P<0.01. Syto9 staining visualized by confocal microscopy confirmed antibiofilm activity of 10% v/v B. subtilis 6D1 CFE (FIG. 1D) compared to an untreated control of S. aureus ATCC 29213 (FIG. 1E). Macroscopic observation of S. aureus ATCC 29213 biofilms grown for 24 hours and washed prior to Syto9 staining (FIG. 1F).

FIGS. 2A-2H illustrate Bacillus subtilis 6D1 cell free extracts (CFEs) that reduce S. aureus ATCC 29213 biofilm growth, disassemble mature biofilm, and improve biofilm inhibition when applied in conjunction with low doses of antibiotics. S. aureus (5×10⁵ CFU/ml) was grown on polystyrene plates at 37 °C for 1, 6, 12, 18, or 24 hours in the presence or absence of B. subtilis 6D1 CFE (10% v/v) (FIG. 2A). S. aureus (5×10⁵ CFU/ml) was grown on polystyrene plates at 37 °C for 0, 1, 6, 12, or 18 h, followed by treatment with B. subtilis 6D1 CFE (10% v/v) at 37 °C for up to 24 h (FIG. 2B). B. subtilis 6D1 CFE (20%, 10%, 5%, or 2.5% v/v) was applied concurrently with S. aureus at TO prior to staining biofilm at 24 h (FIG. 2C). B. subtilis 6D1 CFE (20%, 10%, 5%, or 2.5% v/v) was applied to S. aureus 24 h biofilms and incubated at 37 °C shaking at 100 rpm for 1 hour (FIG. 2D). B. subtilis 6D1 CFE (10% v/v) applied in conjunction with 0.5 μg/ml antibiotic at 0 h prior to measuring S. aureus biofilm growth at 24 h (FIGS. 2E-2G). B. subtilis 6D1 CFE harboring low molecular weight metabolites (<3 kDa) obtained through centrifugal filtration maintain antibiofilm activity against S. aureus ATCC 29213. Bacillus CFE fractions (>30 kDa, <30 kDa, and <3 kDa) obtained via centrifugal filtration were measured for antibiofilm activity to narrow the size range of potentially active compounds (FIG. 2H). (*) P<0.05; (**) P<0.01; (***) P<0.001; (****) P<0.0001.

FIGS. 3A-3B illustrates B. subtilis 6D1 modulates gene expression associated with S. aureus quorum sensing. B. subtilis 6D1 (blue) and 10% v/v B. subtilis 6D1 cell free extract (teal) were applied to S. aureus ATCC 29213 for 24 hours before RNA extraction and expression analysis via RT-qPCR (FIG. 3A). Proposed model of S. aureus QS interference by B. subtilis 6D1, B. subtilis heterologous AIPs bind to AgrC and trigger phosphorylation and activation of DNA binding protein AgrA (FIG. 3B). (*) P<0.05; (**) P<0.01; (***) P<0.001 compared to the untreated control.

FIGS. 4A-4I illustrate whole genome sequencing of B. subtilis 6D1 that reveals potentially unique biosynthetic gene clusters (BCGs). The assembled B. subtilis 6D1 genome (FIG. 4A) was analyzed with AntiSmash 6.0. Phylogenetic analysis comparing B. subtilis 6D1 (highlighted) to 98 similarly identified Refseq genomes using 500 random single copy protein coding genes (FIG. 4B). AntiSmash V6.0 and Clinker were used to visualize and compare reference BGCs (top) to those found in B. subtilis 6D1 (bottom) for subtilosin A (FIG. 4C), sporulation killing factor (FIG. 4D), bacilysin (FIG. 4E), bacillibactin (FIG. 4F), and bacillaene (FIG. 4G). Commercial HPLC grade fengycin obtained from B. subtilis increases S. aureus biofilm formation in a concentration and strain specific manner (FIG. 4H). (*) P<0.05 (**) P<0.01 (***) P<0.001 compared to the control. The assembled B. subtilis 6D1 genome (FIG. 4A) compared to reference biosynthetic gene clusters (FIG. 4I).

FIGS. 5A-5D. B. subtilis 6D1 derived peptides inhibit biofilm growth in all Agr backgrounds and an Agr null derivative. B. subtilis 6D1 ethyl acetate extracts exhibited dose dependent antibiofilm activity against S. aureus strains with different AgrC receptors (agrI-IV) and a strain incapable of producing AgrD (RN7206) (*) P<0.0001 (FIG. 5A). Antibiofilm activity of B. subtilis 6D1 ethyl acetate extracts (500 μg/ml) further separated by CombiFlash fractionation. (***) P<0.001, (****) P<0.0001 (FIG. 5B). LC-MS/MS spectra of B. subtilis 6D1 fraction 100P1 m/z values that matched known molecules in the GNPS database. Black spectra represent peaks identified in fraction P1-100% and green spectra represent GNPS database spectra. The x-axis represents m/z and the y-axis represents the relative abundance of the various ions (FIG. 5C). Uncharacterized sub-network identified in B. subtilis 6D1 fraction 100% P1. LC-MS/MS spectra of B. subtilis 6D1 fraction 100P1 m/z values that did not match any known molecules in the GNPS database; the x-axis represents m/z and the y-axis represents the relative abundance of the various ions (FIG. 5D).

FIGS. 6A-6F illustrate B. subtilis 6D1 100P1 fraction exhibits unique antibiofilm activity compared to commercial HPLC grade surfactin obtained from B. subtilis. DMSO applied up to 5% v/v did not alter S. aureus ATCC 29213 biofilm growth or increase cell death compared to an untreated control. Scale bar=10 μm (FIG. 6A). Antibiofilm activity of titrated concentrations of commercial surfactin and B. subtilis 6D1 fraction 100% P1 against S. aureus ATCC 29213 (FIG. 6B). Live (SYTO9)/dead (PI) staining and confocal image analysis comparing the arithmetic mean fluorescence intensity of S. aureus ATCC 29213 biofilms treated with 500 μg/ml commercial surfactin and B. subtilis 6D1 fraction 100% P1, scale bar=70 μm (FIG. 6C). Antibiofilm activity of commercial surfactin and B. subtilis 6D1 fraction 100% P1 against S. aureus strains with different AgrC receptors (agrI-IV) and a strain incapable of producing AgrD (RN7206) (FIG. 6D). Antibiofilm activity of commercial surfactin and B. subtilis 6D1 fraction 100% P1 against clinical S. aureus (FIG. 6E) and S. epidermidis strains (FIG. 6F). (*) P<0.05; (**) P<0.01; (***) P<0.001; (****) P<0.0001

FIGS. 7A-7F illustrate B. subtilis 6D1 reduces S. aureus ATCC 29213 virulence in a human intestinal cell line. Vero and HT29 cells pretreated for two hours with increasing concentrations of B. subtilis 6D1 CFE reduce cytotoxicity of S. aureus ATCC 29213 (Sa29213) induced cell death; data normalized to Sa29213 positive control (FIG. 7A, FIG. 7D). Cytotoxicity of Vero cells pretreated for two hours with increasing concentrations of fraction 100% P1 and surfactin delivered in DMSO in the presence (FIG. 7B) and absence (FIG. 7C) of a S. aureus ATCC 29213 challenge; data normalized to LDH positive control. Pretreatment of HT29 cells for two hours with B. subtilis 6D1 CFE increases production of the anti-inflammatory cytokine IL-10 (FIG. 7E) and inhibits the production of the pro-inflammatory cytokine TNFα brought on by S. aureus 29213 challenge (FIG. 7F) (*) P<0.05; (**) P<0.01; (***) P<0.001 (****) P<0.0001, ND=not detected.

FIG. 8 is a schematic experimental design investigating the ecological effects of S. aureus ATCC 29213 evolution after long-term exposure to B. subtilis 6D1 derived cell free extracts harboring quorum sensing interference (QSI) activity.

FIGS. 9A-9D illustrate B. subtilis 6D1 CFE maintains antibiofilm activity against all evolved S. aureus populations regardless of treatment or lifestyle. Average population size as determined by CFU/ml across five independently evolved replicates after 1 and 11 days of treatment exposure (FIG. 9A). Average population size as determined by CFU/bead across all five independently evolved replicates after 1 and 11 days of treatment exposure (FIG. 9B). Biofilm growth inhibition quantified using CV staining. 10% v/v B. subtilis 6D1 CFE was applied to S. aureus populations at TO and incubated statically at 37 °C for 24 hours. Residual biofilm biomass was quantified by crystal violet staining and averaged by treatment/lifestyle (C) Biofilm dispersal quantified using CV straining. Either PBS or 10% v/v B. subtilis 6D1 CFE was applied to S. aureus 24-hour biofilms and incubated at 37 °C shaking at 100 rpm for 2 hours. Residual biofilm biomass was quantified by crystal violet staining and averaged by treatment/lifestyle (FIG. 9D). (****) P<0.0001, (***) P<0.001.

FIGS. 10A-10D illustrate B. subtilis 6D1 CFE inhibits S. aureus auto aggregation. Auto-aggregation rate of control lineages in the absence (FIG. 10A) and presence (FIG. 10B) of 10% v/v B. subtilis 6D1 CFE averaged across replicates by treatment/lifestyle. Auto aggregation rate of B. subtilis CFE exposed lineages in the absence (FIG. 10C) and presence (FIG. 10D) of 10% v/v B. subtilis 6D1 CFE averaged across replicates by treatment/lifestyle. (****) P<0.0001, (***) P<0.001, (*) P<0.05.

FIGS. 11A-11B illustrate S. aureus populations evolved in the presence of B. subtilis 6D1 CFE are less competitive in a biofilm environment against B. subtilis 6D1. Planktonic competition outcomes between B. subtilis 6D1 (Bs) and independently evolved S. aureus populations (FIG. 11A). Biofilm competition outcomes between B. subtilis 6D1 and independently evolved S. aureus populations (FIG. 11B). Each circle represents a single population tested in triplicate. Competitions with the ancestor were performed in quadruplicate using four separately prepared S. aureus ATCC 29213 cultures. (***) P<0.001; (**) P<0.01; (*) P<0.05.

FIGS. 12A-12B illustrate S. aureus populations evolved in the presence of B. subtilis 6D1 CFE are less virulent than the ancestor. Vero cell cytotoxicity measured via lactate dehydrogenase release after two-hour infection with S. aureus populations (FIG. 12A) Vero cell morphology observed at 20× magnification after 2 hour exposure to representative S. aureus populations evolved in the presence or absence of Bs CFE (FIG. 12B) Black circles highlight blunted morphologies indicative of unhealthy Vero cells. Independently evolved populations were assayed in triplicate and averaged per treatment-lifestyle. Data were normalized to Vero cell lysis positive control (*) P<0.05.

FIGS. 13A-13M illustrate antibiotic susceptibility testing of S. aureus lineages that reveals treatment and lifestyle-associated phenotypic sensitivity patterns. Kirby Bauer (KB) disc diffusion assays were used to compare antibiotic sensitivities of the S. aureus ATCC 29213 ancestor and evolved populations with nucleic acid synthesis inhibitors Sulfamethoxazole/Trimethoprim (FIG. 13A), Levofloxacin (FIG. 13B), and Rifampin (FIG. 13C), protein synthesis inhibitors Gentamicin (FIG. 13D), Erythromycin (FIG. 13E), and Tetracycline (FIG. 13F), and cell wall synthesis inhibitors Cefotaxime (FIG. 13G), Ceftazidime (FIG. 13H), and Vancomycin (FIG. 13I). (S)usceptible and (R)esistance thresholds as defined by Sensi-disc protocols for Staphylococcus are plotted as dashed lines. Bars represent mean±SD averaged across replicates by treatment/lifestyle. Bars represent mean±SD averaged across replicates by treatment/lifestyle (****) P<0.0001 (***) P<0.001; (**) P<0.01; (*) P<0.05. Media composition also reveals treatment and lifestyle dependent 24-hour growth patterns. Planktonic cell density achieved after 24-hour incubation at 37 °C in modified TSB (FIG. 13J) and MHB (FIG. 13K) averaged across replicates by treatment/lifestyle. (****) P<0.0001. Evolved lineages produce resistant satellite colonies after 72 hours exposure to Rifampin and Vancomycin (FIG. 13L). Mutations and frequency across S. aureus evolved lineages are identified in FIG. 13M.

FIG. 14A-B illustrates population sequencing that reveals treatment and lifestyle-associated molecular targets of S. aureus evolution. Mutations identified by whole-population genome sequencing of S. aureus lineages evolved in the presence and absence of 10% v/v B. subtilis 6D1 CFE revealed these populations were protected from developing mutations in both competence and drug resistance pathways seen in similarly evolved control lineages (FIG. 14A). Five populations per treatment were sequenced after 11 days of experimental evolution. Mutations identified in the evolved populations that were not present in the ancestor are denoted by colored boxes. Shading indicates the frequency of mutation detection in the entire evolved population. Mean plasmid read coverage of each S. aureus population (black dot), normalized to the mean chromosome read coverage and averaged by treatment/lifestyle (FIG. 14B). Differences were analyzed using a non-parametric Kruskal-Wallis test followed by Dunn's multiple comparisons where (**) P<0.01; (*) P<0.05

FIG. 15 depicts missense mutations (red) altering protein residues incurred during S. aureus ATCC 29213 evolution mapped on Alphafold predicted protein structures.

FIG. 16 illustrates plasmid read coverage follows a treatment and lifestyle dependent pattern. Mean plasmid read coverage of each S. aureus population (black dot) was normalized to the mean chromosome read coverage per lineage and averaged by treatment/lifestyle.

FIGS. 17A-D illustrate that B. subtilis 6D1 exhibits antibiofilm activity against S. aureus and harbors unique genetic traits not observed in closely related B. subtilis strains. FIG. 17A shows the antibiofilm activity of 16 Bacillus cell-free extracts against S. aureus 29213. Error bars represent mean±SD of 8 independent replicates. FIG. 17B depicts the assembled B. subtilis 6D1 genome. FIG. 17C illustrates that B. subtilis 6D1 shares >95% identity with a group of B. subtilis genomes (purple) and is more distantly related to common laboratory type strains (dots with arrows). FIG. 17D shows the results from pangenome analysis, which identified 156 unique genes in B. subtilis 6D1 that were not detected in any strain from this closely related group. Singleton gene clusters identified range from 0-233, number of gene clusters identified range from 0-5000.

FIGS. 18A-B illustrate that B. subtilis 6D1 modulates gene expression associated with S. aureus Agr quorum sensing and biofilm development. B. subtilis 6D1 (gray; FIG. 18A) and 10% v/v B. subtilis 6D1 cell-free extract (light gray; FIG. 18A) and B. subtilis 6D1 fraction 100% P1 (light gray; FIG. 18B) and commercial surfactin (dark gray; FIG. 18B) were applied to S. aureus ATCC 29213 for 24 hours before RNA extraction and (Left) Agr associated and (Right) biofilm and stress-associated gene expression analysis via RT-qPCR. Error bars represent mean±SD of 9 independent replicates; Differential gene expression data were analyzed using one-way ANOVA followed by Tukey's multiple comparisons using log 2 transformed data where (*) P<0.05; (**) P<0.01; (***) P<0.001 (****).

FIG. 19 illustrates antimicrobial activity of presumptive Bacillus strain cell-free extracts against S. aureus ATCC 29213. S. aureus (5×105 CFU/mL) was grown shaking at 150 RPM on polystyrene plates at 37 °C for 24 hours in the presence of 6 different Bacillus CFEs applied at 10-20% v/v; (*) P<0.05 compared to the negative control (ATCC 29213).

FIG. 20 illustrates sixteen agriculturally sourced Bacillus strains that do not harbor enterotoxigenic genes found in pathogenic B. cereus and B. thuringiensis species. Mix1-3=Multiplex primer composition; BC=Bacillus cereus; BT=Bacillus thuringiensis; L=Ladder.

FIGS. 21A-B illustrate the impact of molecular inhibitors on the S. aureus antibiofilm activity of B. subtilis 6D1 CFE. FIG. 21A shows biofilm growth inhibition quantified using crystal violet (CV) staining. 10% v/v B. subtilis 6D1 treated CFEs were applied to S. aureus ATCC 29213 at TO and incubated statically at 37 °C for 24 hours. Residual biofilm biomass was quantified by CV staining and averaged across 8 individual experiments FIG. 21B shows biofilm disruption quantified using CV straining. Either PBS or 10% v/v B. subtilis 6D1 treated CFEs were applied to S. aureus ATCC 29213 24-hour biofilms and incubated at 37 °C shaking at 100 RPM for 2 hours. Residual biofilm biomass was quantified by CV staining and averaged across 8 individual experiments. Differences were analyzed using a 2-tailed unpaired t-test where (****) P<0.0001, (***) P<0.001, (**) P<0.01 (*) P<0.05.

FIGS. 22A-B illustrate the minimum inhibitory concentrations of S. aureus ATCC 29213 planktonic cells and biofilms in response to different antibiotics. FIG. 22A shows the minimum biofilm inhibitory concentration (MBIC) of S. aureus ATCC 29213 in 1.5% TSB+0.3% glucose media. Biofilm growth inhibition quantified using crystal violet (CV) staining. Antibiotics were applied to S. aureus at TO and incubated statically at 37 °C for 24 hours. Residual biofilm biomass was quantified by CV staining and averaged across 8 individual experiments. FIG. 22B shows the minimum inhibitory concentration (MIC) of S. aureus ATCC 29213 in 1.5% TSB+0.3% glucose media. Antibiotics were applied to S. aureus and incubated at 37 °C shaking at 150 RPM for 24 hours. Cell density was measured spectrophotometrically and averaged across 8 individual experiments.

FIG. 23 illustrates that HT29 cells treated with B. subtilis 6D1 CFE (20% v/v) and challenged with S. aureus ATCC 29213 exhibit reduced cytotoxicity compared to unchallenged and untreated HT29 cells. HT29 cells pretreated for two hours with B. subtilis 6D1 CFE (20% v/v) inhibit S. aureus ATCC 29213 (Sa29213) induced cytotoxicity as measured by lactate dehydrogenase (LDH) release; data normalized to Sa29213 positive infection control. Differences were analyzed using one-way ANOVA, followed by Tukey's multiple comparisons where (*) P<0.05; (****) P<0.0001.

FIG. 24 illustrates missense mutations acquired during S. aureus ATCC 29213 evolution. Amino acid residues harboring missense mutations (labeled) were mapped onto AlphaFold predicted protein structures and visualized using Pymol.

FIG. 25 illustrates that B. subtilis 6D1 CFE inhibits S. aureus biofilm growth on a polystyrene bead under aerobic and anaerobic conditions. S. aureus 29213 was inoculated into 5 mL 1.5% TSB+0.3% glucose in a glass test tube harboring a single polystyrene bead. These cultures were grown at 37 °C for 24 hours in the presence (triangles) or absence (circles) of 10% v/v B. subtilis 6D1 CFE (Bs CFE) under either aerobic or anaerobic conditions. Beads were removed from the culture media, gently rinsed in PBS, placed into 1 mL sterile PBS, and sonicated at 60 Hz for 10s prior to CFU enumeration. Biofilm growth inhibition was quantified across three independent experiments. Bars represent mean±SD. Differences were analyzed using a 2-tailed unpaired t-test where (****) P<0.0001, (**) P<0.01.

FIG. 26 illustrates potential Applications of B. subtilis 6D1 and B. subtilis 6D1 cell-free extracts.

FIGS. 27A-B illustrate that the unique compound identified in B. subtilis 6D1 fraction 100% P1 inhibits S. aureus biofilm growth and disrupts mature biofilm. FIG. 27A shows biofilm growth inhibition quantified using crystal violet (CV) staining. Either DMSO or B. subtilis6D1 refined fractions were applied to S. aureus populations at TO and incubated statically at 37 °C for 24 hours. Residual biofilm biomass was quantified by CV staining and averaged across three independent experiments. FIG. 27B shows biofilm disruption quantified using CV straining. Either DMSO or B. subtilis6D1 refined fractions were applied to S. aureus 24-hour biofilms and incubated at 37 °C shaking at 100 RPM for 2 hours. Residual biofilm biomass was quantified by crystal violet staining and averaged across three independent experiments. Differences were analyzed using a 2-tailed unpaired t-test where (****) P<0.0001, (***) P<0.001.

FIG. 28 illustrates that flash chromatography further separates the unique compound from the surfactins identified in B. subtilis 6D1 100% P1. LC-MS/MS spectra of B. subtilis 6D1 fraction 100 P1 m/z values (top) comprising two distinct fractions harboring only surfactins (bottom, right shaded region) or only the unique compound (middle, left shaded region). The x axis represents the retention time in minutes and the y axis represents the relative abundance of the various ions.

### DETAILED DESCRIPTION

In the following detailed description, reference is made to the accompanying drawings that form a part disclosure. The embodiments are described in sufficient detail to enable those skilled in the art to practice the invention, and it is understood that other embodiments may be utilized and that changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments are defined by the appended claims.

Unless defined otherwise, all technical and scientific terms used in this disclosure with the appended claims have the same meaning that is commonly understood by one of ordinary skill in art to which the subject matter pertains. Although any methods and materials similar or equivalent to those described herein can be used in the practice for testing of the present invention, the preferred materials and methods are described herein. As used in the specification and the appended claims, unless specified to the contrary, the following terms have the meaning indicated to facilitate the understanding of the disclosure.

All methods described herein can be performed in any suitable order unless otherwise indicated herein or otherwise clearly contradicted by context.

The use of any and all examples, or exemplary language (e.g., “such as”) provided herein is intended merely to illuminate a description or embodiment and does not pose a limitation on the scope of the invention otherwise claimed. No language in the present specification should be construed as indicating any non-claimed element essential to the practice of the invention.

The terms “a,” “an,” “the” and similar references in the disclosure, including in the context of the claims, include both the singular and the plural, unless indicated otherwise or the context clearly indicates otherwise.

Further, ordinal indicators—such as “first,” “second,” “third,” etc.—for identified elements are used to distinguish between the elements, and do not indicate or imply a required or limited number of such elements, and do not indicate a particular position.

Also as used herein, “and/or” refers to and encompasses any and all possible combinations of one or more of the associated listed items, as well as the lack of combinations when interpreted in the alternative (“or”).

Unless stated otherwise, the term “about” means an approximation of a stated value within 10% (e.g., within 5%, 2% or 1%) of the particular value modified by the term “about.”

The term “comprising,” which is synonymous with “including,” “containing,” or “characterized by,” is inclusive or open-ended and does not exclude additional, unrecited elements or method steps, whether essential or not. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “patient” or “subject” refers to all animals, including mammals, preferably humans, who are prone to or are suffering from the indicated disease or disorder, or are treated with the described products or compositions or in accordance with the described methods.

A subject “in need of” treatment in the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

The term “contacting” may be reacting, interacting, or touching, and may include allowing two species to react, interact, or physically touch.

The present disclosure introduces novel extracts, compositions, and methods of using the extracts and composition for countering multidrug-resistant (MDR) pathogens, including anti-biofilm, anti-bacterial, and anti-quorum sensing for MDR pathogens. Anti-biofilm activity includes inhibiting biofilm formation and growth and disassembling mature biofilm formation.

Staphylococcus aureus (S. aureus) is one of the most clinically important multi-drug resistant (MDR) pathogens in the world with infections leading to high rates of morbidity and mortality in both humans and animals. This bacteria's ability to form biofilm, or adherent communities embedded in complex extracellular matrix, protects individual cells from antibiotics and promotes the transfer of antibiotic resistance genes. Therefore, new strategies aimed to inhibit biofilm growth and disassemble mature biofilms have been developed in the disclosed compositions and methods.

Probiotics or probiotic bacteria are known to have the ability to outcompete pathogens and strengthen the immune system. The probiotic-derived compounds from the probiotics responsible for these phenomena are of great interest and largely uncharacterized. Probiotic-derived small molecules, termed postbiotics, are providing an alternative approach to probiotics by both eliminating the perceived concern of administering live bacteria and ensuring greater reproducibility by using a more defined quantity of active compounds. In addition to low toxicity concerns and improved shelf life stability, many postbiotics have defined chemical structures and mechanisms that make them highly therapeutically attractive.

Bacillus subtilis (B. subtilis) is a gram-positive spore forming bacterium with probiotic qualities known to produce antimicrobial compounds. Here, novel probiotic compounds from Bacillus subtilis have been identified with antibiofilm, antibacterial, and anti-quorum sensing activity, particularly against Staphylococcus aureus. In particular, cell-free extracts of B. subtilis containing lipopeptide fractions are found to disrupt quorum sensing and biofilm assembly in S. aureus.

### Extracts and Compositions

One aspect of the disclosure is an antimicrobial extract or composition that includes the extract comprising a B. subtilis 6D1 cell free extract (CFE). An antimicrobial composition may include a B. subtilis 6D1 cell free extract (CFE) and an antibiotic. The B. subtilis 6D1 cell-free extract is a cell-free extract derived from isolated B. subtilis 6D1 culture obtained from agricultural samples. A B. subtilis 6D1 extract is first obtained from the isolated cultures, and then fractioned to obtain a B. subtilis 6D1 cell-free extract that has confirmed anti-biofilm activity. In embodiments, the B. subtilis 6D1 CFE comprises surfactins A-D and a unique compound.

The B. subtilis 6D1 extract may be further separated to isolate the biologically active fraction. In the embodiments, the active fraction has anti-biofilm activity. In the embodiments the active fraction is identified as the B. subtilis 6D1 P1 glycoprotein cell free fraction (B. subtilis 6D1-P1 CFE). In embodiments, the B. subtilis 6D1-P1 CFE is a 75% or 100% P1 glycoprotein cell-free fraction. In some embodiments, the B. subtilis 6D1-P1 CFE is a 100% P1 fraction. In embodiments, the B. subtilis 6D1-P1 CFE comprises surfactins A-D and at least one unique compound (interchangeably referred to herein as “unique compound,” “unique compounds,” or “Compound E”).

In the embodiments, the B. subtilis 6D1-100% P1 CFE is further separated to isolate additional biologically active fractions. In embodiments, the active fraction comprises surfactins, such as surfactins A-D, or Compound E. In embodiments, Compound E, or the active fraction comprising Compound E, has antibiofilm activity, including preventing formation of biofilm, interrupting biofilm formation, inhibiting biofilm growth, or disassembling/disrupting mature biofilm. In some embodiments, Compound E, or the active fraction comprising Compound E, has potent activity in inhibiting biofilm growth and disassembling/disrupting mature biofilm.

The term “isolated,” when applied to a bacterium, refers to a bacterium that has been (1) separated from at least some of the components with which it was associated when initially produced (whether in nature or in an experimental setting), and/or (2) produced, prepared, purified, and/or manufactured by the hand of man, e.g. using artificial culture conditions such as (but not limited to) growing on a plate and/or in a fermenter. Isolated bacteria include those bacteria that are cultured, even if such cultures are not monocultures. Isolated bacteria may be separated from at least about 10%, about 20%, about 30%, about 40%, about 50%, about 60%, about 70%, about 80%, about 90%, about 95%, about 96%, about 97%, about 99% or more of the other components with which they were initially associated (e.g., by weight).

The B. subtilis 6D1 CFE has anti-biofilm activity. In the embodiments, the B. subtilis 6D1 CFE has anti-biofilm activity in a pathogenic bacterium, such as S. aureus. In the embodiments, the antibiofilm activity may prevent formation of biofilm, interrupt biofilm formation, inhibit biofilm growth, or disassemble/disrupt mature biofilm. In embodiments, the pathogenic bacterium is resistant to antibiotics.

The B. subtilis 6D1 CFE has anti-biofilm activity but does not inhibit growth of the pathogenic bacterium or kill the pathogenic bacterium. Rather than killing a pathogen cell, antibiofilm extract or compound produced from B. subtilis 6D1 disrupts or interferes with the bacterial communication system among the cells, including the ability to sense cell density or quorum. As a result, a pathogen, such as S. aureus, abandons biofilm formation and maintenance processes. This mechanism of disrupting or interfering with biofilm formation and maintenance, referred to as quorum sensing interference, may be used to control the virulence of the biofilm forming pathogens without directly killing them. In embodiments, quorum sensing interference or anti-quorum sensing activity inhibits biofilm activity in pathogens. In embodiments, the Bacillus subtilis 6D1 CFE inhibits bacterial quorum sensing activity in a pathogenic bacterium, such as S. aureus. As a result of quorum sensing interference in S. aureus, antibiotic sensitivity of S. aureus is improved.

In the embodiments, the anti-quorum sensing activity is the accessory gene regulator (Agr) quorum sensing interference mechanism. In embodiments, the Bacillus subtilis 6D1 cell free extract increased expression of agrA, RNAIII, and hld genes in the S. aureus operon. The increased expression, or upregulation, of the genes transition cells from a biofilm lifestyle to a planktonic lifestyle. In embodiments, the B. subtilis 6D1 CFE upregulates the global stress response regulator genes sigB, and saeR, thus decreases virulence of S. aureus.

As used herein in all aspects and embodiments, B. subtilis 6D1 CFE includes biologically active fractions thereof, such as an active fraction comprising Compound E.

A further aspect is a composition that includes a B. subtilis 6D1 CFE and an antibiotic. Combining B. subtilis 6D1 CFE extract with antibiotic provides compositions with antibacterial activity, along with anti-biofilm activity. In embodiments, the composition inhibits growth of a pathogenic bacterium, such as S. aureus.

In embodiments, the antibiotic is selected from Ampicillin, Gentamicin, and Trimethoprim, or combinations of s antibiotics. The antibiotic may be a concentration suitable for the application of the composition, which may be readily determined by one of skill in the art. In embodiments, the dose of antibiotic is a low dose in a range from about 0.5 ug/ml to about 5 ug/ml sufficient for antibacterial effect on the pathogen of interest. The dose of antibiotic may be about 0.5 μg/ml, 0.6 μg/ml, 0.7 μg/ml, 0.8 μg/ml, 0.9 μg/ml, 1 μg/ml, 1.5 μg/ml, 2 μg/ml, 2.5 μg/ml, 3 μg/ml, 3.5 μg/ml, 4 μg/ml, 4.5 μg/ml, 5 μg/ml. In some embodiments, the dose of antibiotic is 0.5 μg/ml.

Another aspect is directed to a pharmaceutical composition that includes a therapeutically effective amount of the disclosed compositions or B. subtilis 6D1 CFE, or pharmaceutically effective amount or pharmaceutically acceptable amount of the composition or B. subtilis 6D1 CFE with a pharmaceutically acceptable carrier. The terms “effective amount,” “effective dose,” “therapeutically effective amount,” “pharmaceutically effective amount”, etc. refer to the amount of an agent that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of pathogen or diseased cells.

The term or phrase “pharmaceutically acceptable” means that which is useful in preparing a pharmaceutical composition that is generally safe, non-toxic and neither biologically nor otherwise undesirable and includes that which is acceptable for veterinary or human pharmaceutical use. The term or phrase “pharmaceutically acceptable carrier,” as known in the art, refers to a pharmaceutically acceptable material, medium, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, tissue, or cell, or portion of the body, to another organ, or portion of the body. A carrier is “acceptable” in the sense of being physiologically inert to and compatible with the other ingredients of the formulation and not injurious to the subject or patient.

The disclosed compositions may be in various form, such as solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). The disclosed compositions may be formulated for pharmaceutical use. The disclosed compositions may also be formulated for rapid, intermediate, or extended release.

### Methods

Another aspect of the disclosure is a method of obtaining the B. subtilis 6D1 cell-free extract. Natural Bacillus subtilis cultures may be isolated from the environment. The isolated Bacillus subtilis cultures are processed to separate the cells and cellular material to obtain a cell-free extract. The cell-free extracts may be separated to obtain cell-free extract biologically active fractions. In embodiments, the cell-free extracts may be separated with ethyl acetate to obtain fractionated extract, that is, fractionated B. subtilis 6D1 cell free extract, with anti-biofilm activity. In embodiments, the method further includes purifying the B. subtilis 6D1. Any method of purification of a biological extract may be used in the methods.

The fractionated B. subtilis 6D1 cell free extract may be further separated to obtain the active P1 glycoprotein extract of B. subtilis. Thus, a Bacillus subtilis 6D1 CFE is obtained and fractionated to a B. subtilis 6D1-P1 CFE with confirmed anti-biofilm activity. In the embodiments, the Bacillus subtilis 6D1 CFE P1 fraction is a 75% to 100% P1 glycoprotein cell-free fraction. In some embodiments, the method further includes purifying the fractionated B. subtilis 6D1-P1 CFE.

In some embodiments, the B. subtilis 6D1-100% P1 CFE may be further separated or purified to isolate additional biologically active fractions. In embodiments, the active fraction comprises surfactins, such as surfactins A-D, or Compound E. In embodiments, Compound E, or the active fraction comprising Compound E, has antibiofilm activity, such as preventing formation of biofilm, interrupting biofilm formation, inhibiting biofilm growth, or disassembling/disrupting mature biofilm. In some embodiments, Compound E, or the active fraction comprising Compound E, produces greater disruption of mature biofilm as compared to a fraction that includes surfactins.

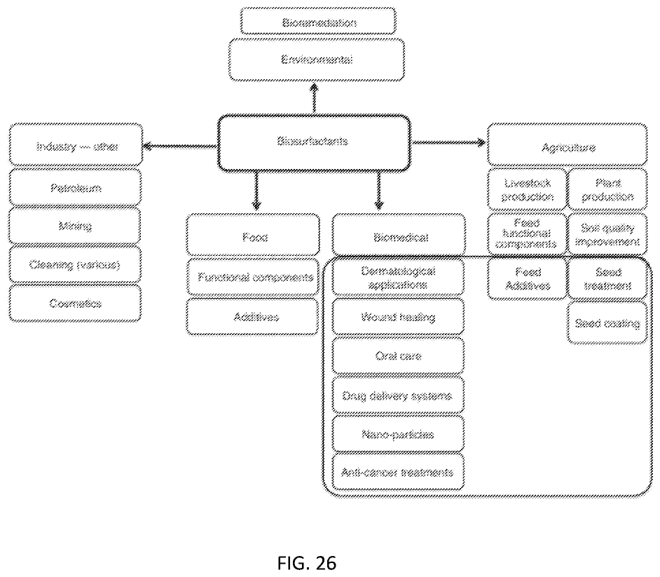

Further aspects are methods of using the disclosed B. subtilis 6D1 CFE or compositions containing B. subtilis 6D1 CFE. One aspect is a method of inhibiting biofilm activity in a pathogenic bacterium, such as S. aureus, by delivering or administering the disclosed compositions or B. subtilis 6D1 cell-free extract to a pathogenic bacterial (e.g. S. aureus) infestation or infection environment. In embodiments, the environment may be any environment that may harbor an infestation or infection with S. aureus, such as agricultural, biomedical including pharmaceutical/clinical, and food environments. Examples of such environments or applications for the disclosed compositions may be used are illustrated in FIG. 26.

As used herein, “infestation” refers to a microorganism on an object or external to an object, which may not be penetrated by the microorganism. The microorganism may be a pathogen.

As used herein, “infection” refers to a microorganism in an object or on an object that may be penetrated by the microorganism, including on or in a subject's body. The microorganism may be a pathogen.

As used herein, the term “infectious environment” refers to the place, area, object etc. that may have an infection by a microorganism such as a bacterium, such as a pathogenic bacterium. An infectious environment may be on, in, around or surrounding a subject or object.

In embodiments, the composition may be delivered or administered directly to the pathogenic (e.g., S. aureus) infestation or infection environment.

In the embodiments, inhibiting biofilm activity includes preventing formation of biofilm, interrupting biofilm formation, inhibiting biofilm growth, or disassembling mature biofilm.

A disclosed composition or B. subtilis 6D1 cell-free extract can be used in methods in any environment with a S. aureus formation, infestation, or infection. Such environments may be biomedical (pharmaceutical/clinical) or agricultural environments. The compositions or B. subtilis 6D1 cell-free extract may be applied to agricultural, medical or clinical devices, for example, such as a coating, film, or infused material (e.g., infused into gauze, bandages, or patches). The compositions or B. subtilis 6D1 cell-free extract may be formed as an antibacterial material to be applied to an infestation, infection, infected area or environment, such as antibacterial powders, ointments, pastes, creams, lotions, gels, solutions (including in which a compound is dissolved, or contains liposomes, lipoplexes, micelles, or nanoparticles), suspensions, emulsions, copolymers, hydrogels, drops, sprays, inhalants, and patches. These compositions can help to reduce S. aureus colonization and thus the risk of recurrent and chronic infestations or infections.

Another aspect is a method of using the disclosed B. subtilis strain 6D1, extracts and compositions for treating a S. aureus infestation or infection by administering to an object or subject an effective amount of B. subtilis 6D1 cell-free extract, or a composition comprising the extract, or administering B. subtilis strain 6D1. In embodiments, the S. aureus is resistant to antibiotics. Such methods include a method of treating a disease or disorder characterized by resistance to antibiotics. The methods include a method of treating a subject having a S. aureus infection characterized by resistance to antibiotics. Such disease or disorder may include a methicillin susceptible Staphylococcus aureus (MSSA) or methicillin resistant Staphylococcus aureus (MRSA) conditions. The MSSA or MRSA may produce a biofilm. In the methods, the MSSA or MRSA biofilm activity is inhibited or destroyed.

An embodiment is a method of treating a S. aureus infection in a subject by administering to the subject an effective amount of B. subtilis 6D1 cell-free extract, or a composition comprising a B. subtilis 6D1 cell-free extract. In embodiments of the method, S. aureus is resistant to antibiotics. In embodiments of the method, biofilm activity in the S. aureus is inhibited. In some embodiments of the method, quorum sensing in the S. aureus infection is inhibited, and/or virulence of the S. aureus is reduced. In some embodiments of the method, resistance to antibiotics in the S. aureus is reduced or eliminated.

Another embodiment is a method of using the disclosed B. subtilis strain 6D1, extracts and compositions in treating a S. aureus infection or infestation by delivery or administering an effective amount of B. subtilis 6D1 cell-free extract, a composition comprising the extract, or a B. subtilis strain 6D1. In embodiments, delivery or administration may be to a medical/clinical or agricultural environment or device. In embodiments, the S. aureus is resistant to antibiotics. In embodiments of the method, biofilm activity in the S. aureus is inhibited. In some embodiments of the method, quorum sensing in the S. aureus infection is inhibited, and/or virulence of the S. aureus is reduced. In some embodiments of the method, resistance to antibiotics in the S. aureus is reduced or eliminated.

In all embodiments, the disclosed bacteria, extracts, and compositions may be delivered or administered by application directly or to a surface or physical object. Compounds, compositions, or formulations may be administered to a subject or an object using any amount and any route of administration that may be effective for preventing, diagnosing, or treating a disease, disorder, condition, infection, or infestation. Modes of administration may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, intradermal, intravaginal, intraperitoneal, mucosal, nasal, intratracheal instillation, bronchial instillation, and inhalation), local, and topical (e.g., transdermal and intradermally). Extracts, compositions, or formulations may be in the form of powders, tablets, dispersible granules, capsules, cachets, solutions (including in which a compound or extract is dissolved, and solutions containing liposomes, lipoplexes, micelles, or nanoparticles), suspensions (including in which solid material is dispersed), emulsions, syrups, elixirs, tinctures, gels, creams, lotions, copolymers, and hydrogels, sprays, and aerosol. They may also be formulated for rapid, intermediate, or extended release. The types of extracts, compositions, or formulations for local or topical administration are typically in the form of powders, ointments, pastes, creams, lotions, pastes, gels, solutions suspensions, emulsions, drops, sprays, inhalants, and patches. In general, the most appropriate route of administration will depend upon a variety of factors including, for example, the nature of the agent (e.g., its stability in the environment), and/or the condition of the subject (e.g., whether the subject is able to tolerate administration). In the embodiments, the disclosed extracts or compositions may be delivered or administered by a delivery agent, for example to a subject using a pharmaceutically acceptable carrier.

In the disclosed methods, S. aureus fitness, virulence, and/or resistance to antibiotics may be reduced.

### EXAMPLES

The invention can be further understood in view of the following non-limiting examples.

The discovery and subsequent industrial manufacturing of antimicrobial compounds revolutionized healthcare in the 20th century. However, overuse and misuse of these antimicrobials has led to an increase in bacterial antimicrobial resistance (AMR), a serious public health threat. According to the World Health Organization, AMR accounted for an estimated 700,000 deaths worldwide in 2019, a number that is expected to surpass 10 million by 2050 if immediate measures are not taken(7). In 2019, 2.5 million AMR infections cost the Unites States economy more than $55 billion, roughly $580M of which is attributed directly to methicillin susceptible and methicillin resistant Staphylococcus aureus infections (1,8). Although antibiotics have helped prevent and treat a variety of diseases over the last 70 years, bacterial pathogens have evolved unique strategies to develop resistance to these compounds, prompting new efforts to investigate antibiotic alternatives (9). Antibiotic alternatives are broadly defined as any substance that can prevent or reduce the need for antimicrobial drugs—these can include vaccines, phytochemicals, organic acids, and other non-disease-causing bacteria.

The use of live beneficial bacteria, commonly referred to as probiotics, can control pathogens directly through competitive interactions or indirectly by stimulating the host immune response(9), and these products have been shown to prevent and treat a variety of diseases(10). Though the majority of probiotic research has sought to identify strains with broad bactericidal activity (11), recent evidence suggests some strains can reduce disease by inhibiting pathogen quorum sensing, a bacterial communication system that modulates gene expression and lifestyle selection. Quorum sensing inhibitors do not kill bacteria; instead, they interfere with communication systems needed to form biofilm and produce virulence factors. Furthermore, quorum sensing interference (QSI) has also been proposed as a method to improve antibiotic killing(12,13), making it an intriguing mechanism with immediate therapeutic potential. Exploring anti pathogenic mechanisms that reduce biofilm formation and improve antibiotic efficacy is essential to understand how probiotics can be leveraged to combat multi-drug resistant (MDR) pathogens and reduce the spread of antimicrobial resistance.

Antibiotic resistant S. aureus is a leading cause of pneumonia, sepsis, endocarditis, and soft tissue infections—made more difficult to treat due to its biofilm formation abilities and its evolving resistance to front-line antibiotics(14) and its ability to form biofilm(106, 107). According to the National Institutes of Health, approximately 60% of all bacterial infections are caused by biofilm formation, an aggregated heterogenous community of cells that increases antibiotic resistance 1000-fold(4) and facilitates the transfer of antibiotic resistance genes(3). Therefore, developing novel therapeutic and prophylactic strategies to prevent and eradicate biofilm formation for MDR pathogens like S. aureus is critical.

Staphylococcus aureus is one of the most clinically important MDR pathogens in the world with infections leading to high rates of morbidity and mortality in both humans and animals. The Staphylococcus aureus bacteria's ability to form biofilm, or adherent communities embedded in complex extracellular matrix, protects individual cells from antibiotics and promotes the transfer of antibiotic resistance genes. S. aureus is a major colonizer of skin infections—aided primarily by its ability to form biofilm(106,107). Within biofilm communities, bacteria compete with their neighbors for space and resources, and it has been reported that these competitive environments increase S. aureus virulence and antibiotic resistance(97). Furthermore, biofilm environments combine high cell density and high selection pressure increasing the rate with which resistant cells appear through mutations or gene transfer. Therefore, if biofilm is disassembled or inhibited, it stands to reason that virulence within that community also decreases(18,75). However, over evolutionary time, host-specific S. aureus has jumped the species barrier between livestock and humans in both directions several times(109), suggesting adaptations that occur during carriage may be functionally important for pathogenesis(110). Therefore, new strategies aimed to inhibit biofilm growth and disassemble mature biofilms are urgently needed.

The majority of antibiofilm compounds described previously either inhibit biofilm or disassemble mature biofilm. Often, these compounds are also bactericidal, forcing pathogens to adapt and evolve resistance to these compounds, rendering them clinically non-viable in a short period of time. Probiotics are now gaining traction for their ability to outcompete pathogens and strengthen the immune system, however the probiotic derived compounds responsible for these phenomena are largely uncharacterized.

In S. aureus, quorum sensing (QS) is controlled through the secretion and self-recognition of autoinducing peptides. Specifically, the agrBDCA operon encodes the AgrD precursor of the autoinducing-peptide (AIP), which is modified and secreted by AgrB. AIPs then bind to AgrC and trigger phosphorylation and activation of DNA binding protein AgrA, driving the expression of the agr regulon via binding to the P2 and P3 promoters. This process allows S. aureus to coordinate complex behaviors, including the formation and disassembly of biofilm. Though these peptides are intended to serve as strain specific cues, it has been shown that peptides produced by taxonomically different species can bind to AgrC and successfully initiate the quorum sensing pathways in neighboring cells(15,16), a strategy that may be employed to reduce biofilm formation and improve antibiotic efficacy(17-19).

Approximately 90% of S. aureus infections involve skin and soft tissues(20) and a functioning Agr QS system is required to produce these human infections(21). The Agr QS system is conserved among S. aureus strains, but variations within AgrD and the C-terminus of AgrB result in four different mature AIPs that are uniquely recognized by four different AgrC configurations (22). Each of the four S. aureus Agr groups has a different biological consequence: Agr-I is linked to enterotoxin disease; Agr-II is linked to early vancomycin-resistance and endocarditis; Agr-III is linked to endocarditis and menstrual toxic shock syndrome; and Agr-IV is linked to exfoliative disease(23,24). Despite these differences, the Agr QS system in each group is responsible for regulating virulence factor gene expression(25) and facilitating biofilm formation and dispersal(26,27). When the Agr system is turned on, S. aureus maintains a planktonic lifestyle; conversely when Agr is turned off, S. aureus forms biofilm. Importantly, competitive Agr signaling interference is sufficient to abrogate this phenomenon(17).

Quorum-sensing interference (QSI) has been touted as an ideal strategy to mitigate gram positive and gram negative bacterial infections(12, 94). Unlike traditional antibiotics, which mainly aim to inhibit and kill microorganisms, QS inhibiting agents do not pressure the growth of microorganisms, nor are they likely to induce the development of antibiotic resistance(13, 95), as they are likely to target nonessential genes and impose reduced selective pressure minimizing the probability of resistance development. Furthermore, QS coordinates the production of many important extracellular factors that are cooperative “public goods” for the population. Mutants that do not produce these goods, but benefit from them, are considered social cheaters. Under conditions that favor QS, such cheaters emerge in the form of receptor-negative, signal-blind mutants. They invade wild-type populations with negative frequency dependence as their proportion increases in a population, their relative fitness decreases as there are fewer cooperators to exploit(111). This suggests social cheating may play an important role in reducing the development of resistance to QSI and, perhaps more importantly, also suggest that QSI-resistant mutants would be unable to enrich during infection. Based on these genetic and ecological mechanisms, it has been assumed QSI is much less prone to induce resistance adaptations in the bacterial populations in which they are applied. However, resistance mechanisms against well-characterized quorum quenchers (QQs) have been found in the laboratory as well as in clinical strains, demonstrating that the rise of resistance against these kinds of compounds is possible(96).

Though a variety of synthetic compounds have been investigated for their ability to reduce S. aureus biofilm growth(28-30), very few studies have explored the QSI potential of natural bacterial products to control S. aureus virulence and biofilm formation. In recent years, the use of probiotics has shown extraordinary potential to prevent and treat a variety of diseases(31), including S. aureus infections(32). Probiotics possess a variety of strain specific strategies to reduce pathogen growth, virulence, and/or biofilm formation. Furthermore, interactions with probiotic compounds can increase pathogen sensitivity to antibiotics and improve pathogen recognition by the host immune system(6).

One such probiotic species, Bacillus subtilis, is gaining interest as a potential therapeutic against S. aureus for its ability to produce an array of antimicrobials and signaling molecules capable of reducing S. aureus biofilm growth and virulence. Though the potential health benefits associated with B. subtilis are well documented, the reproducibility of these interventions largely depends on the B. subtilis strain used(33,34)—complicated by each strain's unique genetic composition and antimicrobial production potential. The number of sequenced bacterial genomes has increased exponentially over the last two decades(35), revealing extraordinary strain level genetic differences and useful information regarding potentially new secondary metabolites with novel mechanisms of action. B. subtilis strains are remarkably diverse and shaped both by their environment and their ability to acquire genes from closely related species(36,37) therefore, due to B. subtilis' diverse ecological range, it is perhaps not surprising that strain specific genetic elements have evolved to help this species compete in a variety of environments(38,39). This species excretes a variety of compounds(40) that inhibit S. aureus growth(41) and can disrupt S. aureus quorum sensing(42), however since the majority of these experiments use crude Bacillus cell free extracts, the precise compounds driving these phenomenon go largely unidentified. Here, we sought to investigate how Bacillus inhibits S. aureus biofilm growth, identify the compounds driving this activity, and assess these compounds' protective effects against S. aureus infection in eukaryotic cell culture experiments. Identifying B. subtilis compounds that mitigate S. aureus biofilm formation and virulence will expand our understanding of the utility of probiotic therapies and may improve how these products are applied to inhibit MDR bacterial infections, improve antibiotic efficacy, and diminish the severity of AMR.

Probiotic bacterial species, namely B. subtilis, is gaining interest as a potential therapeutic against S. aureus for its ability to produce an array of antimicrobials and signaling molecules (40) capable of reducing S. aureus colonization in humans (79). However, probiotic strains that display antagonistic behaviors incur a significant energy cost and are at increased risk of injury and death(78). Conversely, bacteria that succumb to competition quickly adapt to overcome these bactericidal threats, often increasing their resistance to antibiotics in the process. Hence, aggression is not without risk; competitive interactions between bacteria, even clonal populations, are a driving force behind antibiotic resistance evolution(97). Interestingly, B. subtilis possesses an ability to identify and discriminate between closely related strains prior to initiating gene transfer events(112) and targeted antimicrobial production(113), suggesting social interactions within this species can override mechanistic barriers to horizontal gene transfer. In fact, competition against B. subtilis significantly slows down the selection for E. coli antibiotic resistance not due to competition for resources, but by the extracellular compounds produced by B. subtilis(29). It's also been determined multiple peptides produced by this species act synergistically to effectively eliminate phylogenetically distinct competitors(113). QSI strategies provide new possibilities to overcome this issue, affording probiotic bacteria a competitive advantage without killing or inhibiting the growth of competitors, and thereby reducing the likelihood of antibiotic cross resistance.

Though the potential health benefits associated with B. subtilis are well documented, the reproducibility of these interventions largely depends on the B. subtilis strain used(33,34)—complicated by each strain's unique genetic composition and antimicrobial production potential. The number of sequenced bacterial genomes has increased exponentially over the last two decades(35), revealing extraordinary strain level genetic differences and useful information regarding potentially new secondary metabolites with novel mechanisms of action. B. subtilis strains are remarkably diverse and shaped both by their environment and their ability to acquire genes from closely related species(36,37) therefore, due to B. subtilis' diverse ecological range, it is perhaps not surprising that strain specific genetic elements have evolved to help this species compete in a variety of environments(38,39). This species excretes a variety of compounds(40) that inhibit S. aureus growth(41) and can disrupt S. aureus quorum sensing(42), however since the majority of these experiments use crude Bacillus cell free extracts, the precise compounds driving these phenomena go largely unidentified.

Here, Bacillus strains capable of inhibiting S. aureus biofilm growth via QS interference, and the compounds driving this activity are identified, and these strains' protective effects against S. aureus in eukaryotic cell culture experiments assessed. Identifying B. subtilis compounds that interfere with S. aureus communication systems will expand our understanding of the utility of probiotic therapies and may improve how these products are applied to inhibit MDR bacterial infections, improve antibiotic efficacy, and diminish the severity of AMR.

Bacillus strains (1123 strains) obtained from a variety of agricultural environments were collected and screened in search of isolates with strong antibiofilm activity against clinical MDR S. aureus. The disclosed results have identified a single probiotic strain, B. subtilis 6D1, that produces novel antibiofilm compounds and possesses unique abilities to inhibit biofilm growth, disassemble mature biofilm, and improve antibiotic sensitivity of S. aureus biofilms through a quorum sensing interference mechanism. A sample of the B. subtilis 6D1 strain was deposited with the US Department of Agriculture Agricultural Research Service (ARS) Culture Collection (NRRL, Peoria, Illinois) 9 Mar. 2023, and assigned NRRL accession number B-68257.

Genomic analysis of this Bacillus strain revealed unique biosynthetic gene clusters. LC-MS/MS and molecular networking analysis of an active organic fraction 100% P1 revealed the presence of surfactin A-D and unique compounds which may be driving this antibiofilm activity. Furthermore, this active B. subtilis 6D1 100% P1 organic fraction/cell free extract harbored greater antibiofilm activity against S. aureus species when compared to commercial HPLC grade surfactin obtained from B. subtilis, suggesting the presence of these novel compounds are contributing to the antibiofilm activity observed. Cell culture results further revealed compounds produced by B. subtilis 6D1 reduce S. aureus virulence in a Vero and HT29 human intestinal cell lines.

Quorum sensing in Staphylococcus aureus is an internal population dependent signaling system responsible for virulence factor production and lifestyle selection, therefore, quorum sensing interference (QSI) has been touted as an ideal mechanism for the development of new anti-infective therapies. Recent studies have established Bacillus subtilis, a gram-positive spore forming bacterium with probiotic qualities, produces metabolites capable of inhibiting S. aureus infection via quorum sensing interference. However, it remains unknown how long-term exposure to these molecules drive S. aureus adaptation and evolution.

B. subtilis 6D1, a strain previously shown to inhibit S. aureus virulence and biofilm formation via QSI, was used in this study. S. aureus ATCC 29213 was evolved in the presence of B. subtilis 6D1 cell free extracts (CFE), and daily passages of planktonic cells and biofilms were performed for 80 generations. Fitness, virulence, and antibiotic resistance assays of the ancestor and all evolved populations revealed the emergence of treatment and lifestyle associated ecological traits. Compared to the ancestor, S. aureus populations evolved in the presence of B. subtilis 6D1 CFE were less competitive in a biofilm, less cytotoxic in a Vero cell infection model, and exhibited increased phenotypic sensitivity to multiple antibiotics. Notably, B. subtilis 6D1 CFE maintained its ability to inhibit S. aureus biofilm growth and disassemble mature biofilm by way of deterring auto aggregation in all lineages. Variant analyses determined S. aureus populations evolved in the presence of B. subtilis 6D1 CFE harbored new junctions spanning plasmid borne efflux systems and missense mutations in genes associated RNA polymerase function but were protected from developing mutations in both competence and drug resistance pathways found in similarly evolved control lineages. The data suggests long term exposure to probiotic-derived molecules, like those produced by B. subtilis 6D1, can reduce S. aureus' fitness and virulence without creating cross resistance to multiple antibiotics.

### Materials and Methods

The following materials and methods were used for the experiments herein unless otherwise indicated, such as in Example 8.

Bacterial Strains and Chemicals Used in this Study

TABLE 1
Bacterial Strains

| Strain Name | Reference |
|---|---|
| S. aureus ATCC 29213 | American Type |
| | Culture Collection |
| S. aureus RN6734 (Prototypical agr-I) | Ji et al. 1997 |
| S. aureus RN6607 (Prototypical agr-II) | Ji et al. 1997 |
| S. aureus RN3984 (Prototypical agr-III) | Ji et al. 1997 |
| S. aureus RN4850 (Prototypical agr-IV) | Ji et al. 1997 |
| S. aureus RN7206 (agr-null | Ji et al. 1997 |
| derivative of RN6734) | |
| S. aureus RSM5 | This Paper |
| S. aureus RSM97 | This Paper |
| S. aureus RSM256 | This Paper |
| S. aureus RSM424 | This Paper |
| S. aureus RSM486 | This Paper |
| S. epidermidis ATCC 12228 | American Type |
| | Culture Collection |
| S. epidermidis RSM78 | This Paper |
| S. epidermidis RSM133 | This Paper |
| S. epidermidis RSM242 | This Paper |
| S. epidermidis RSM268 | This Paper |
| S. epidermidis RSM425 | This Paper |
| B. subtilis NCIB 3610 | Bacillus Genetic |
| | Stock Center |
| B. subtilis 9B5 | This Paper |
| B. subtilis 6D1 | This Paper |
| B. pumilus 11D12 | This Study |
| B. pumilus 10D6 | This Study |
| B. pumilus 11G1 | This Study |
| B. pumilus 11F9 | This Study |
| B. pumilus 11B2 | This Study |
| Bacillus 11A10 | This Study |
| Bacillus 11A12 | This Study |
| Bacillus 3A3 | This Study |
| Bacillus 10C11 | This Study |
| Bacillus 2G11 | This Study |
| Bacillus 11G5 | This Study |
| Bacillus 3H6 | This Study |
| Bacillus 1E1 | This Study |
| Bacillus 5A12 | This Study |

Table 2. Antibiotic Susceptibility Testing of S. aureus Clinical Isolates Used to Assess Bacillus CFE Antibiofilm Activity.

| Staph | | | | | | | | | | | | | | | |
|---|---|---|---|---|---|---|---|---|---|---|---|---|---|---|---|
| aureus | | | | | | | | | | | | | | | |
| ID | Source | Cef | Gen | Lev | Sxt | Pen | Ery | Clin | Lzd | Van | Tet | Dox | Dap | Tel | Dal |
| 5917 | WD | S | S | R | S | S | I | S | S | S | S | S | S | S | S |
| 5918 | WD | S | S | I | S | S | S | S | S | S | S | S | S | S | S |
| 5919 | WD | S | S | R | S | S | R | R | S | S | S | S | S | S | S |
| 5920 | WD | R | S | S | S | R | R | S | S | S | S | S | S | S | S |
| 5921 | TI | S | S | S | S | R | S | S | S | S | S | S | S | S | S |
| 5922 | WD | S | S | S | S | R | S | S | S | S | S | S | S | S | S |
| 6014 | WD | S | S | S | S | R | I | S | S | S | S | S | S | S | S |
| 6015 | WD | R | S | R | S | R | R | R | S | S | S | S | S | S | S |
| 6016 | WD | S | S | S | S | R | S | S | S | S | S | S | S | S | S |
| 6017 | ABSC | S | S | S | S | R | R | S | S | S | S | S | S | S | S |
| 6018 | WD | S | S | S | S | S | I | S | S | S | S | S | S | S | S |
| 6019 | WD | S | S | S | S | R | R | S | S | S | S | S | S | S | S |
| 6020 | WD | S | S | S | S | S | S | S | S | S | S | S | S | S | S |
| 6021 | WD | R | S | S | S | R | R | S | S | S | S | S | S | S | S |
| 6533 | WD | R | S | R | S | R | R | S | S | S | S | S | S | S | S |
| 6535 | WD | R | S | R | S | R | R | R | S | S | S | S | S | S | S |
| 6536 | WD | S | S | S | S | S | S | S | S | S | S | S | S | S | S |
| 6537 | WD | R | S | S | S | R | S | S | S | S | S | S | S | S | S |
| 6538 | WD | R | S | S | S | R | S | S | S | S | S | S | S | S | S |
| 6539 | WD | S | S | S | S | R | R | S | S | S | S | S | S | S | S |
| 6540 | WD | S | S | S | S | S | R | R | S | S | S | S | S | S | S |
| 6541 | WD | S | S | S | S | R | S | S | S | S | S | S | S | S | S |
| 6565 | TI | R | S | R | S | R | R | S | S | S | S | S | S | S | S |
| 6566 | WD | S | S | R | S | S | R | R | S | S | S | S | S | S | S |
| 6645 | WD | R | S | R | S | R | R | S | S | S | S | S | S | S | S |
| 6647 | WD | S | S | S | S | R | R | S | S | S | S | S | S | S | S |

WD: Wound;
ABSC: Abcess;
TI: Titanium implant;
Gen: Gentamicin;
Tet: Tetracycline;
Pen: Penicillin;
Rif: Rifampin;
Ery: Erythromycin;
Sxt: Sulfamethoxazole/Trimethoprim;
Cef: Cefoxitin;
Lev: Levofloxacin;
Van: Vancomycin;
Clin: Clindamycin;
Lzd: Linezolid;
Dox: Doxycycline;
Dap: Daptomycin;
Tel: Telithromycin;
Dal: Dalvabancin
Antibiotic Susceptibility Designations: S-sensitive;
R-resistant;
I-intermediate resistant

Surfactin from Bacillus subtilis >98% (CAS: 24730-31-2) (Lot: 0000134293) (Source: 0000128758), Fengycin from Bacillus subtilis ≥90% (CAS: 102577-03-7) (Source: SLCK5954), and Lysostaphin (CAS: 9011-93-2) (Lot: 109M4010V) were obtained from Sigma-Aldrich (USA). Dimethyl Sulfoxide (CAS: 67-68-5) 611 (Lot: 184176) used to deliver peptides and as a comparable vehicle control was purchased from Fisher Chemical, USA.

### Bacillus Candidate Screening

To attain presumptive Bacillus isolates, approximately 50 environmental samples comprised of bovine feces and milk, and manure contaminated river sediment and soil, were collected. One gram per sample was applied to 9 mL 0.1% peptone, heated to 72 °C for 20 minutes to induce Bacillus sporulation and plated aerobically in tryptic soy agar (TSA). To ensure only a single strain was obtained, a total of 1123 presumptive Bacillus colonies were individually picked with sterile toothpicks and inoculated into fresh square TSA plates in a 6×6 grid. To reduce overgrowth from any fastidious strains, plates were grown at room temperature for 48 hours. After incubation, each strain was sub-cultured on agar plates to ensure pure culture isolation and subsequently in 500 μL trypticase soy broth (TSB) in 96-well deep v-well reservoirs (Thermo Scientific, USA). All cultures were grown for 24 hours at 37 °C and used immediately for antimicrobial screening assays. A total of 1123 presumptive Bacillus colonies were sub-cultured in tryptic soy broth (TSB) and stored at −80 °C.

All Bacillus strains were assessed for antagonistic activity against methicillin resistance and methicillin susceptible clinical and type strains of S. aureus using drop diffusion overlay and cross streak assays (43). For drop diffusion assays, all 1123 presumptive Bacillus isolates were grown for 24 hours in TSB at 37 °C and 5 μl were spotted onto square petri plates (VWR International, USA) with TSA media in a 6×6 grid fashion and allowed to dry. Seven mL of soft TSA (0.75% agar) harboring 150 μL of exponential phase S. aureus cultures (~2.5×108 CFU/mL) was overlaid onto the TSA plate and allowed to dry. Plates were then inverted and incubated at 37 °C for 18 hours before measuring clearing zones indicative of antagonistic activity. A total of 75 strains exhibiting inhibition against both S. aureus strains were subsequently assayed using cross streak methods. Briefly, 100 μL of each presumptive Bacillus culture was inoculated into 10 mL TSB and incubated overnight at 37 °C. A loop full of each culture was struck vertically down the center of a square TSA plate and incubated for an additional 16 hours at 37 °C. The next day, nine different methicillin susceptible and methicillin resistant clinical and type strains, including the two used previously for drop diffusion assays, were struck perpendicularly towards the Bacillus streak, allowed to dry, and incubated at 37 °C overnight prior to measuring zones of inhibition. An additional library of Staphylococcus strains isolated from the milk of 27 different dairy cows diagnosed with mastitis were used to assess antagonistic activity of candidate Bacillus strains.

Bacillus strains (n=46) exhibiting broad antagonistic activity against S. aureus (Table 3) were individually cultured in 125 mL baffled shake flasks containing 25 mL TSB, and incubated at 37 °C shaking at 230 RPM for 48 hours. After incubation, cultures were centrifuged at 4 °C at 10,500 RPM for 20 minutes prior to harvesting cell free extracts (CFE) for use in subsequent biofilm and planktonic inhibition assays. CFEs were harvested by filter-sterilization using a 0.22 μm filter (Corning, USA). CFEs were snap frozen in liquid nitrogen and stored at −80 °C for future use.

TABLE 4
Antagonistic activity of 16 Bacillus isolates against microbial pathogenic type strains

| | | | | | Cross Streak Inhibition (mm) | | | | | | | | | | | |
|---|---|---|---|---|---|---|---|---|---|---|---|---|---|---|---|---|
| | | | | | C. | C. | | | | | | S. | | L. | | |
| | | Lib- | | | per- | per- | C. | | | | S. | epi- | S. | mono- | E. | C. |
| | | rary | | | fri- | fri- | diffi- | C. | C. | E. | typhi- | der- | grami- | cyto- | fae- | albi- |
| | Plate | loca- | | WGS | ngens | ngens | cile | coli | jejuni | coli | murium | midis | nearum | gens | calis | cans |
| Site | Loc. | tion | 16S ID | ID | 3626 | S107 | 9689 | 33559 | 33560 | 23 | 14028 | 12228 | 46779 | 35152 | 19433 | Y477 |
| OKW2 | C1 | 1:E1 | B. pumilus/ | | 2 | 20 | 1 | 2 | 2 | 2 | 0 | 11 | 9 | 8 | 0 | 2 |
| | | | B. | | | | | | | | | | | | | |
| | | | zhangzhou | | | | | | | | | | | | | |
| | | | ensis | | | | | | | | | | | | | |
| | F5 | 2:G11 | B. pumilus | | 2 | 15 | 2 | 0 | 0 | 3 | 0 | 15 | 7 | 18 | 0 | 2 |
| KRS | C3 | 3:A3 | B. pumilus | | 7 | 30 | 7 | 0 | 0 | 20 | | 14 | 19 | 9 | 2 | 2 |
| CREVICE | E6 | 3:H6 | B. pumilus | | 6 | 28 | 21 | 0 | 0 | 3 | 0 | 14 | 19 | 14 | 2 | 5 |
| | F6 | 5:A12 | B. pumilus | | 3 | 21 | 1 | 3 | 0 | 0 | 0 | 11 | 17 | 8 | 0 | 8 |
| SCKR | C1 | 6:D1 | B. subtilis | B. | 13 | 30 | 8 | 7 | 5 | 2 | 0 | 3 | 19 | 5 | 1 | 19 |
| | | | | subtilis | | | | | | | | | | | | |
| | | | | subsp | | | | | | | | | | | | |
| | | | | subtilis | | | | | | | | | | | | |
| | E5 | 9:B5 | B. subtilis | | 15 | 30 | 16 | 7 | 3 | 2 | 0 | 11 | 16 | 5 | 0 | 5 |
| | F5 | 10:C11 | B. pumilus/ | | 11 | 24 | 0 | 0 | 0 | 1 | 0 | 17 | 14 | 4 | 3 | 3 |
| | | | B. | | | | | | | | | | | | | |
| | | | zhangzhou | | | | | | | | | | | | | |
| | | | ensis | | | | | | | | | | | | | |
| DDDS-3 | A6 | 10:D6 | B. pumilus | B. | 10 | 30 | 10 | 0 | 0 | 2 | 0 | 14 | No | 7 | 2 | 0 |
| | | | | pumilus | | | | | | | | | gro- | | | |
| | | | | | | | | | | | | | wth | | | |
| | F4 | 11:A10 | B. pumilus | | 5 | 4 | 2 | 0 | 0 | 3 | 0 | 20 | 13 | 9 | 3 | 9 |
| | F6 | 11:A12 | B. safensis/ | | 20 | 11 | 3 | 0 | 0 | 4 | 0 | 13 | 0 | 10 | 4 | 3 |
| | | | B. subtilis | | | | | | | | | | | | | |
| MANURE2 | A2 | 11:B2 | B. pumilus | B. | 7 | 18 | 3 | 0 | 0 | 4 | 0 | 16 | 20 | 13 | 2 | 4 |
| | | | | pumilus | | | | | | | | | | | | |
| | F6 | 11:D12 | B. pumilus | B. | 2 | 30 | 21 | 0 | 0 | 4 | 0 | 16 | 19 | 10 | 2 | 10 |
| | | | | pumilus | | | | | | | | | | | | |
| | D3 | 11:F9 | B. pumilus | B. | 10 | 30 | 2 | 0 | 0 | 1 | 0 | 19 | 17 | 16 | 1 | 3 |
| | | | | pumilus | | | | | | | | | | | | |
| | E1 | 11:G1 | B. pumilus | B. | 11 | 30 | 12 | 0 | 0 | 2 | 0 | 15 | 16 | 9 | 2 | 3 |
| | | | | pumilus | | | | | | | | | | | | |
| | E5 | 11:G5 | B. aerius | | 14 | 18 | 4 | 0 | 0 | 2 | 0 | 11 | 14 | 2 | 0 | 0 |

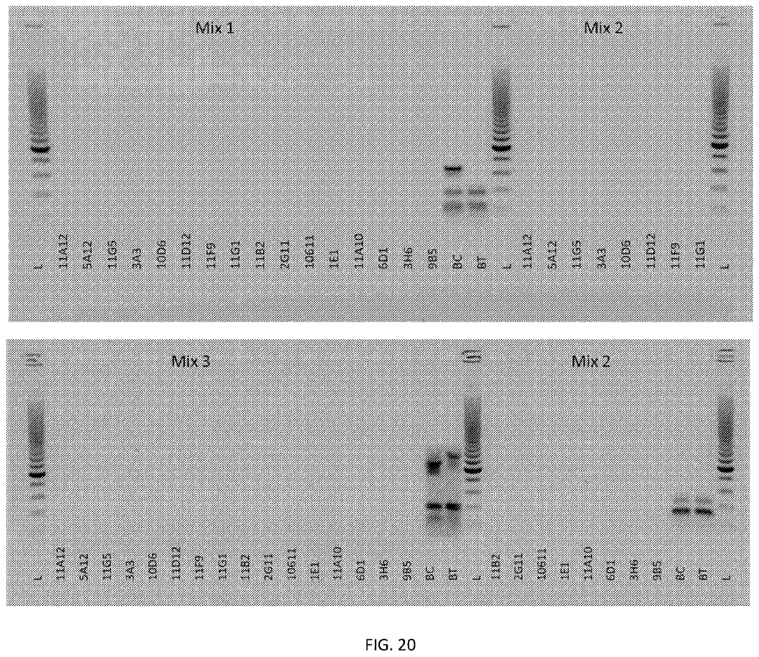

Bacillus CFEs were subjected to additional separation using Amicon® centrifugal filters (Thermo Scientific, USA) and re-evaluated for activity. A total of 39 clinical methicillin susceptible and methicillin resistant S. aureus strains were used to assess antibiofilm activity of Bacillus CFEs. Bacillus strains (n=16) exhibiting broad antimicrobial potential in a planktonic or biofilm environment were assayed for relevant pathogenic Bacillus toxin genes (44) prior to sequencing the 16S rRNA region for taxonomic classification (FIG. 20). A multiplex endpoint PCR assay adapted from Yang et al was used to identify pathogenic Bacillus toxin genes (Yang et al. 2005). Bacillus DNA was isolated from 16 candidate Bacillus strains using DNeasy Blood and Tissue Kit (Qiagen, Germany) according to the manufacturer's instructions. The final PCR reaction mixture (60 μL total volume) included 200 μM deoxynucleoside triphosphates (dNTPs), 80-700 nM primer concentrations (see Table 5), 25 mM MgCl2, 2U of FastStart™ Taq DNA Polymerase (Roche, Switzerland), and approximately 20 ng of template DNA. PCR was performed on an Applied Biosystems 2720 Thermal Cycler (Thermo Scientific, USA) according to the following cycling conditions: 95 °C for 5 minutes, 30 cycles of (95 °C for 30s, 60 °C for 30s, 72 °C for 45s), and final extension 72 °C for 7 min. The resulting PCR product was analyzed on a 2% agarose gel using SyberSafe Gel Stain at 1 μL/10 mL molten agarose. Gels were run for 180 min at 100V. B. cereus (BC) and B. thuringiensis (BT) genomic DNA were used as positive controls.

TABLE 5
Primer pairs for Bacillus enterotoxin assay

| | | Primer | | Concentration | Fragment |
|---|---|---|---|---|---|
| Mix | Targeted gene | name | Sequence(5′ to 3′) | (nM) | size (BP) |
| 1 | Hemolysin BL | HA-F1 | ATT AAT ACA GGG GAT | 150 | 237 |
| | subunit A | | GGA GAA ACT T | | |
| | (hbLA) | HA-R1 | TGA TCC TAA TAC TTC | | |
| | | | TTC TAG ACG CTT | | |
| | Hemolysin BL | HC-F1 | CCT ATC AAT ACT CTC | 240 | 386 |
| | subunit C | | GCA ACA CCA AT | | |
| | (hbLC) | HC-R1 | TTT TCT TGA TTC GTC | | |
| | | | ATA GCC ATT TCT | | |
| | Hemolysin BL | HD-F1 | AGA TGC TAC AAG ACT | 240 | 436 |
| | subunit D | | TCA AAG GGA AAC TAT | | |
| | (hbLD) | HD-R1 | TGA TTA GCA CGA TCT | | |
| | | | GCT TTC ATA CTT | | |
| | Emetic strain- | EM-F1 | AGC TTG GAG CGG AGC | 500 | 635 |
| | specific | | AGA CTA TGT | | |
| | sequence | EM-R1 | GTA TTT CTT TCC CGC | | |
| | | | TTG CCT TTT | | |
| 2 | Nonhemolytic | NA-F1 | ATT ACA GGG TTA TTG | 150 | 475 |
| | enterotoxin | | GTT ACA GCA GT | | |
| | subunit A | NA-R1 | AAT CTT GCT CCA TAC | | |
| | (nheA) | | TCT CTT GGA TGC T | | |
| | Nonhemolytic | NB-F1 | GTG CAG CAG CTG TAG | 180 | 328 |
| | enterotoxin | | GCG GT | | |
| | subunit B (nheB) | NB-R1 | ATG TTT TTC CAG CTA | | |
| | | | TCT TTC GCA AT | | |
| | Nonhemolytic | NC-F1 | GCG GAT ATT GTA AAG | 450 | 557 |
| | enterotoxin | | AAT CAA AAT GAG GT | | |
| | subunit B (nheC) | NC-R1 | TTT CCA GCT ATC TTT | | |
| | | | CGC TGT ATG TAA AT | | |
| 3 | Enterotoxin FM | Fm-F1 | CAA AGA CTT CGT AAC | 160 | 290 |
| | (entFM) | | AAA AGG TGG T | | |
| | | Fm-R1 | TGT TTA CTC CGC CTT | | |
| | | | TTA CAA ACT T | | |
| | Enterotoxin T | Bcet- | AGC TTG GAG CGG AGC | 700 | 701 |
| | (bceT) | F1 | AGA CTA TGT | | |
| | | Bcet- | GTA TTT CTT TCC CGC | | |
| | | R1 | TTG CCT TTT | | |
| | Cytotoxin K | Cyt-F1 | ATC GGG CAA AAT GCA | 500 | 800 |
| | (cytK) | | AAA ACA CAT | | |
| | | Cyt-R1 | ACC CAG TTT GCA GTT | | |
| | | | CCG AAT GT | | |
| | Cytotoxin K | Cyt-F2 | ATC GGT CAA AAT GCA | 500 | 800 |
| | (cytK) | | AAA ACA CAT | | |
| | | Cyt-R2 | ACC CAG TTA CCA GTT | | |
| | | | CCG AAT GT | | |
| ALL | Internal | ITS-F1 | AAT TTG TAT GGG CCT | 80 | 185 |
| | transcribed | | ATA GCT CAG CT | | |
| | spacer | ITS-R1 | TTT AAA ATA GCT TTT | | |
| | | | TGG TGG AGC CT | | |

### Competition Assays

Planktonic and biofilm competitions were adapted from methods outlined by Marshall et al (45,46). Briefly, for planktonic competitions, 250 μl (5×10⁷ CFU) of exponential phase B. subtilis 6D1 and S. aureus populations were added to 25 ml TSB in a baffled shake flask and incubated at 37 °C shaking at 200 RPM. Biofilm competitions utilized 7 mm polystyrene beads as a surface for cell attachment, biofilm growth, and biofilm dispersal (47). For biofilm competitions, equal concentrations of B. subtilis 6D1 and S. aureus populations were added to a glass test tube containing 5 ml 1.5% TSB+0.3% glucose media (48) and a single polystyrene bead. After 24 hours incubation at 37 °C in a roller drum, the bead was removed and placed into PBS, and sonicated for 10 seconds at 60 Hz to remove attached cells. For all competitions, CFU counts were obtained for both bacterial species at time point 0 and after 24 hours incubation. Strain fitness and selection rate were calculated as previously described by Lenski and colleagues (49).

### Biofilm Inhibition and Disruption Assays

Bacillus CFEs were screened by being applied at varying concentrations to Nunc™ treated 96 well microplates (Thermo Scientific, USA) containing 1.5% TSB+0.3% glucose (48). Test wells were seeded with 5×10⁵ CFU/ml of S. aureus and incubating statically at 37 °C. After 24 hours, media was removed, and biofilms were gently washed three times with PBS, fixed for 20 minutes at 55 °C, and stained with 0.1% crystal violet (CV) for 15 minutes. After 15 minutes, CV was removed, and wells were washed twice with PBS and allowed to dry. CV was solubilized with 30% acetic acid for 20 minutes. Biofilm inhibition was measured spectrophotometrically relative to untreated control wells at OD₅₉₅. To assess biofilm dispersal, 10% v/v B. subtilis 6D1 CFE was mixed with PBS and applied to S. aureus 24-hour mature biofilms and incubated at 37 °C shaking at 100 rpm for 2 hours. To account for any mechanical dispersal not driven by CFE, biofilms treated only with PBS were used as a negative control. Residual biofilms were fixed and quantified by crystal violet staining as described above. Each lineage was assayed in quintuplicate and averaged by treatment/lifestyle. Biofilm inhibition and removal were confirmed via CFU plating after the initial PBS wash but prior to any CV staining.

### Antibiotic Synergy Biofilm Inhibition Assays

Bacillus CFEs (Bacillus CFEs including B. subtilis 6D1 CFE) exhibiting antibiofilm potential were applied in conjunction with Ampicillin, Gentamicin, and Trimethoprim, three antibiotics with different mechanisms of action. Checkerboard assays and fractional inhibitory concentration indices were used to assess whether coadministration of antibiotic and Bacillus CFE was synergistic (≤0.5), additive (0.5-1.0), or indifferent (1-4)(43). Briefly, 5×10⁵ CFU/ml S. aureus ATCC 29213 was inoculated in polystyrene 96-well plates and treated with either 10% v/v Bacillus CFE, antibiotic (either Gentamicin, Ampicillin, or Trimethoprim), or a combined antibiotic+Bacillus CFE treatment. Antibiotic concentrations were based on preliminary biofilm and planktonic minimum inhibitory concentrations (MIC) concentrations and recommended CLSI breakpoints (50). Each antibiotic or antibiotic+Bacillus CFE combination was added at time point 0 and biofilm growth was measured via CV staining after 24 h at 37 °C incubation.

### Confocal Laser Scanning Microscopy

To assess the impact of B. subtilis 6D1 compounds on S. aureus biofilm formation, ibidi 8-μwell coated plates (ibidi, Germany) containing 400 μl 1.5% TSB+0.3% glucose were used to grow and subsequently stain residual biofilms. Bacterial viability staining was performed using the Live/Dead BacLight Bacterial Viability Kit (Thermo Scientific, USA) according to manufacturer's instructions and measured via confocal laser scanning microscopy (CLSM) on a Nikon Eclipse Ti instrument. Images and mean fluorescence intensity were quantified in Imaris (version 9.9).

### Whole Genome Sequencing & Bioinformatics Analysis

Whole genome sequencing was performed on B. subtilis 6D1 and five other Bacillus strains with S. aureus biofilm inhibition activity ranging from 15-85%. Results are shown below in Table 6. Briefly, Bacillus DNA was isolated using DNeasy Blood and Tissue Kit (Qiagen, Germany) according to the manufacturer's instructions. To improve sequencing accuracy at the nucleotide level, both Illumina (400 Mbs) and Nanopore (300 Mbs) sequencing was performed. Quality control and adapter trimming was performed with bcl2fastq (51) and porechop (52) for Illumina and Nanopore sequencing respectively. Hybrid read assembly was performed with Unicycler (53), and assembly statistics were recorded with QUAST (54). Assemblies were annotated with Prokka and genome maps were created using Proksee (55). Assemblies were also analyzed in AntiSmash 6.0 to search for unique biosynthetic gene clusters (BGCs) (56). Unique BGCs were further compared to reference BGCs using Clinker (57).

TABLE 6
Whole genome assembly metrics of six Bacillus strains

| | | Bacillus | Bacillus | Bacillus | Bacillus | Bacillus | Bacillus |
|---|---|---|---|---|---|---|---|
| | | 11B2 | 11F9 | 11D12 | 10D6 | 11G1 | 6D1 |
| Genome | Contigs | 1 | 11 | 2 | 3 | 2 | 2 |
| Statistics | Genome | 3757204 | 3765980 | 3929244 | 4215245 | 3802702 | 4156213 |
| | Length | | | | | | |
| | GC Content | 41.720547 | 41.621624 | 41.53458 | 41.34023 | 41.569942 | 43.65195 |
| | Contig L50 | 1 | 1 | 1 | 1 | 1 | 1 |
| | Contig N50 | 3757204 | 3679676 | 3923259 | 4201122 | 3796932 | 4072344 |
| Annotation | tRNA | 81 | 80 | 81 | 81 | 80 | 88 |
| Statistics | rRNA | 24 | 24 | 24 | 24 | 24 | 30 |
| | CDS | 3876 | 3904 | 4173 | 4636 | 3948 | 4309 |
| | CDS Ratio | 1.0316182 | 1.0366491 | 1.0620364 | 1.0998174 | 1.0382092 | 1.036761 |
| | Hypothetical | 825 | 841 | 1015 | 1358 | 876 | 708 |
| | CDS | | | | | | |
| | Hypoth- | 0.38054696 | 0.38114753 | 0.40186915 | 0.4374461 | 0.38652483 | 0.380599 |
| | etical | | | | | | |
| | CDS | | | | | | |
| | Ratio | | | | | | |
| | PLFAM | 3655 | 3658 | 3901 | 4209 | 3689 | 4074 |
| | CDS | | | | | | |
| | PLFAM | 0.94298244 | 0.9369877 | 0.9348191 | 0.90789473 | 0.93439716 | 0.945463 |
| | CDS Ratio | | | | | | |
| Genome | Coarse | 98.5 | 98.3 | 98.5 | 98.2 | 98.3 | 99.7 |
| Quality | Consistency | | | | | | |
| | Fine | 97.1 | 96.9 | 97.1 | 96.7 | 96.9 | 98.6 |
| | Consistency | | | | | | |
| | Genome | Good | Good | Good | Good | Good | Good |
| | Quality | | | | | | |

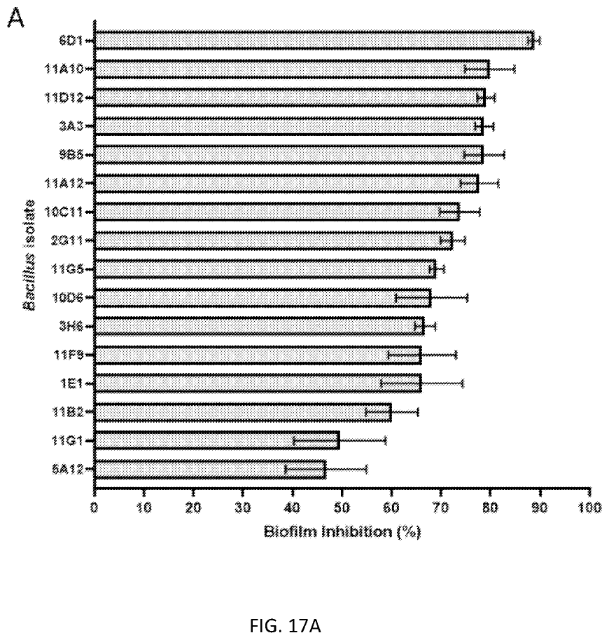

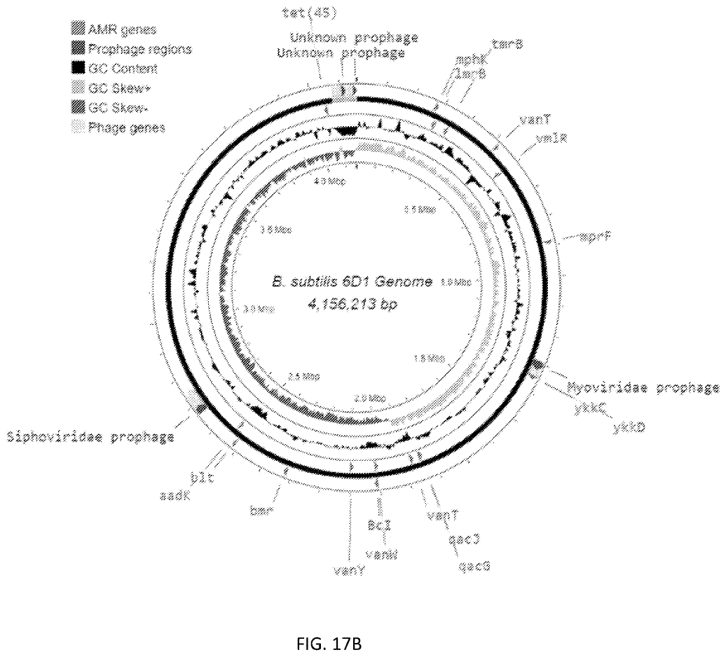

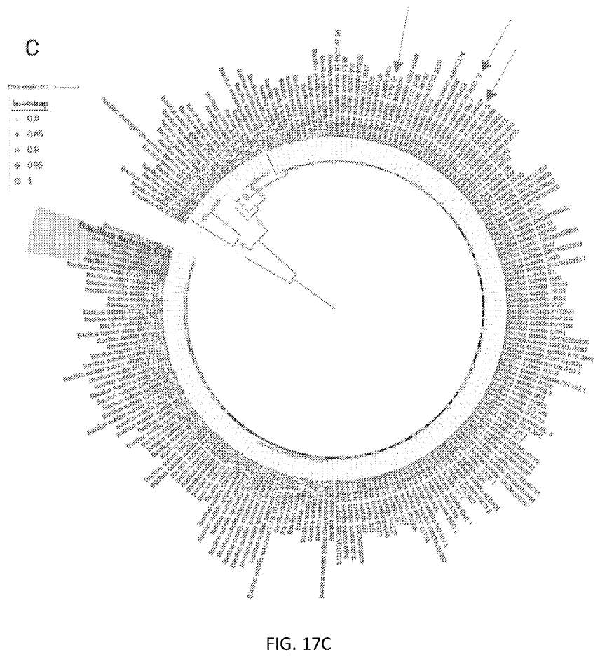

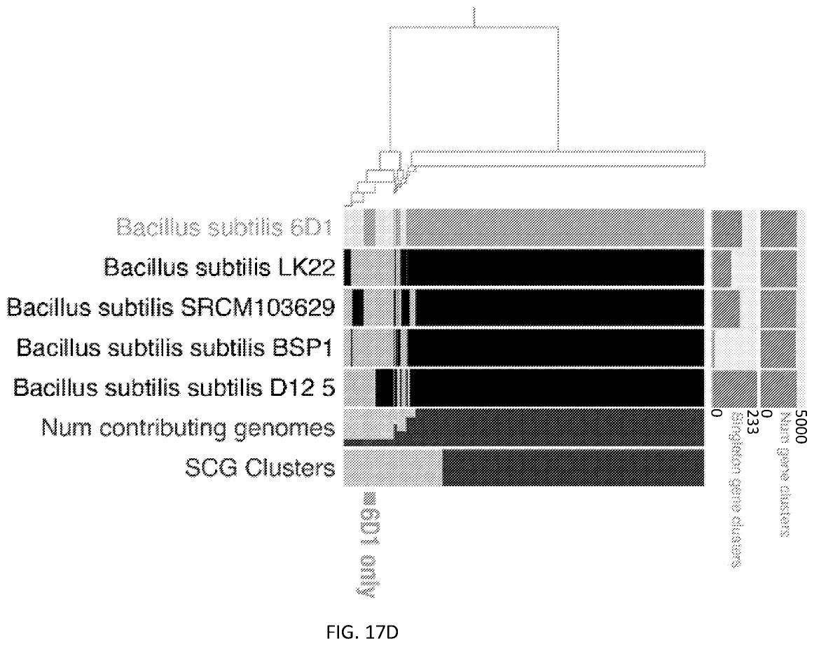

Phylogenetic analysis comparing B. subtilis 6D1 to 98 similarly identified Refseq genomes using 500 random single copy protein coding genes was performed using Minhash similar genome finder and codon tree builder pipelines found in the Bacterial and Viral Bioinformatics Resource Center (BV-BRC). All available genomes of Bacillus subtilis on the IMG database were downloaded and phylogenomic analysis was performed using Anvi'o 6.2 (174). Specifically, we annotated the single copy core genes in all the genomes, concatenated a set of 71 core genes and aligned them in Anvi'o. The phylogenomic tree was constructed according to an approximately-maximum-likelihood method as implemented in FastTree 2.1.10 (175) using the Jones-Taylor-Thorton model. The phylogenomic tree was edited using iTOL (176). Pangenome analyses utilized only genomes that were most closely related to B. subtilis 6D1 in the phylogenomic tree (shaded region in FIG. 17C). Pangenome analysis was performed in Anvi'o as follows: a genome storage database was generated comprised of the B. subtilis 6D1 assembly and each of the closely related genomes; the pangenome analysis was run using the following parameters: minbit=05 and a mcl-inflation=0.9 for the clustering. Diamond was used to compare the genes in sensitive mode (177). The pangenome was visualized, inspected, and edited in Anvi′o. Annotation of unique B. subtilis 6D1 genes was performed in EggNOG-mapper v2.1.12.

16S rRNA Sequencing of Bacillus Candidates

Identifying the genus and species of an unknown organism is an important step in determining if a bacterial strain is AAFCO (Association of American Feed Control Officials)-approved for feed use in livestock production or GRAS (generally recognized as safe)-approved for human consumption. Determining the sequence of the 16S rRNA gene is one method commonly used to determine genus and species of unknown bacterial isolates. The 16S rRNA gene was amplified from high quality genomic DNA isolated from each Bacillus isolate using primers targeting the nucleic acid residues 8 and 1492 (Table 6). All PCR reactions (50 μL) contained 25 μL SYBR Green supermix (Bio-Rad Laboratories, USA) 21 μL molecular grade water, 1 μL of 10 μM working forward and reverse primer concentrations, and 2 μL template DNA. PCR was performed on a BioRad CFX96 real time instrument (Bio-Rad Laboratories, USA) according to the following cycling conditions: 95 °C for 5 min for initial denaturation, 30 cycles of (95 °C for 30s, 51 °C for 30s, 72 °C for 2 min), final extension at 72 °C for 7 min. Melt curve analysis ranged from 65-95 °C in 0.5 °C increments, for 5 seconds. The resulting PCR product was cleaned up to remove leftover dNTPs, Taq polymerase, and impurities with the Invitrogen Purelink PCR purification kit (Thermo Scientific, USA) according to manufacturer's instructions and sent to Eurofins for sequencing. Forward and reverse 16S rRNA reads were aligned using MUSCLE prior to querying sequences using NCBI Blast.

TABLE 7
Primer sequences for amplification of bacterial 16s rRNA gene.

| | | Position on | Approx. | |
|---|---|---|---|---|
| Primer | | E. coli | amplicon | |
| Number | Sequence (5′-3′) | genome | size (bp) | Note |
| Bac-16S-F | CGGTTACCTTGTTACGACTT | 1492 | ~1484 | Primer also |
| | | | | referred to as |
| | | | | 1492R |
| Bac-16S-R | AGAGTTTGATCCTGGCTCAG | 8 | ~1484 | Primer also |
| | | | | referred to as |
| | | | | 8F |

### Bacillus CFE Preparation for LC-MS/MS Analysis

To extract and separate lipopeptides from crude Bacillus CFE, liquid-liquid extractions using ethyl acetate were performed as previously described (58-60). Briefly, Bacillus strains cultured in TSB for 48 hours were centrifuged (4300 RPM, 30 min) at 4 °C. Three liters of culture supernatant were mixed in a 1:1 volume ratio (v/v) with ethyl acetate and allowed to sit overnight with occasional mixing. Ethyl acetate fractions were collected using a separatory funnel and dried under vacuum in a Speed Vac™ rotary evaporator (Thermo Scientific, USA) at 32 °C. Metabolites were resuspended in DMSO and filtered through a 0.45 μm Durapore™ filter (Millipore, USA). Extracts were reassessed for antibiofilm activity prior to LC-MS/MS analysis. Briefly, 100 μl solubilized ethyl acetate extracts were mixed with 15 μl of 2% acetonitrile and 0.1% formic acid prior to 10 μl injections on a 25 cm×75 μm column. MS/MS data was collected on charge states 1-6 using an Orbitrap analyzer and mined for unique metabolite structures using molecular networking methods (61).

### Separation of Ethyl Acetate Organic Extracts Using Flash Chromatography

To simplify the ethyl acetate extract, flash chromatography was performed on a Teledyne Isco CombiFlash NextGen 100, fitted with a C18 column. The extract was dissolved in 25% methanol and injected onto the column. A stepwise gradient of water (A) and methanol (B) from 15% B, 25% B, 50% B, 75% B, to 100% B was used to separate the extract. Fractions were collected using the automatic fraction collector. During the stepwise gradient, individual peaks were collected separately from the rest of the flow through to provide a fraction enriched with those compounds. These peaks were labeled as X % P1 for the first peak separated in any given fraction. Isolated peaks and fractions were combined and dried in vacuo. To further separate 100% P1, this fraction (221 mg) was separated on a reversed-phase open column (RediSep Rf C18 50 g; 60 to 100% aq. methanol) to generate two subfractions containing only surfactins (100% P1-A) or only the unique compound (100% P1-B). Fraction 100% P1-B (142 mg) was purified using a Dionex UltiMate 3000 HPLC system, using a Luna C18 column (250 mm×15 mm, Phenomenex), running acetonitrile with 0.1% formic acid and H₂O with 0.1% formic acid as the mobile phase.

### LC-MS/MS Analysis

Fractions and isolated peaks were dried in vacuo, resuspended in 50% methanol, and analyzed on a Q-Exactive quadrupole orbitrap mass spectrometer (Thermo Scientific, USA) coupled to a UPLC (Dionex). The UPLC method was 5% methanol for 0.5 minutes followed by a linear gradient from 5% methanol to 97% methanol over 16 minutes. The 97% methanol wash was held for two minutes before switching back to 5% methanol over 0.5 minutes and re-equilibrating at 5% methanol for 1 minute. The UPLC flow rate was 0.35 mL/min over a Phenomenex Kinetex XB-C18 chromatography column with dimensions 2.1×100 mm and particle size 2.6 micron. The mass spectrometer scanned from 200 to 2000 m/z in positive mode and ion fragmentation was achieved using stepped normalized collision energy of 30, 35 and 40%. The data was collected in profile mode and manually inspected and filtered using MzMINE2. MzMINE2 was used to create an aligned feature list and quantification table of the mass spectral data of the biologically active peak. These files were used to perform feature-based molecular networking through GNPS(61). The GNPS spectral libraries were searched for matches to submitted MS/MS spectra with a cosine score threshold of 0.7 and minimum of 6 matched peaks to the library spectra. The resulting network file was visualized and analyzed using Cytoscape v3.8.0. All LC-MS/MS data were deposited in the MassIVE database under project number MSV000092675 operated by the Center for Computational Mass Spectrometry at UC-San Diego.

### Gene Expression

RNA was isolated from 24 hour cultures seeded with 5×10⁵ CFU/ml S. aureus ATCC 29213 co-inoculated with either B. subtilis 6D1 cell free extract (10% v/v), 5×10⁵ CFU/ml B. subtilis 6D1 cells (1:1), commercial grade surfactin (500 μg/mL), or B. subtilis 6D1 100% P1 fraction (500 μg/mL) using a Qiagen RNeasy Mini kit adapted to include a modified lysis step with lysostaphin(62). Prior to RNA extraction, each treated culture was mixed 1:2 with RNA Protect (Qiagen, Germany), vortexed for 10 seconds, and incubated at ambient temperature for 10 minutes. 200 μL of lysis buffer (30 mM Tris, 1 mM EDTA, 20 mg/mL lysozyme, 100 μg/mL lysostaphin, pH=8) and 20 μL proteinase K were applied to each culture and incubated at ambient temperature on a rotary shaker (Fisher Scientific, USA) for 45 minutes. RNA quality and quantity were determined with a Qubit spectrophotometer (Thermo Scientific, USA). cDNA was obtained using the Qiagen Quantitect reverse transcriptase kit according to the manufacturer's instructions (Qiagen, Germany). qPCR conditions were optimized and evaluated on a BioRad CFX96 real time instrument (Bio-Rad Laboratories, USA) based on previous work(29). RT-qPCR reaction volumes (20 μl) were comprised of 10 ng cDNA, 300 nM forward and reverse primers, and SYBR green master mix (Bio-Rad Laboratories, USA). The real-time cycling conditions were as follows: 95 °C for 10 min, followed by 40 cycles of 95 °C for 15 seconds and between 58.5-60 °C (based on primer melting temperature) for 1 minute. Gene expression was quantified relative to the rpoB gene(29,63) and calculated using the Livak method(64). RNA expression was determined using three independent cultures, and all analyses were performed in triplicate. Gene primers and their associated melting temperatures are listed in Table 8.

TABLE 8
Gene Primers

| Gene | Forward primer (5′-3′) | Reverse primer (5′-3′) | Tm |
|---|---|---|---|
| agrA | TGCGAAGACGATCCAAAAC | TTTAGCTTGCTCAAGCACCTC | 60 °C |
| aur | GATGGTCGCACATTCACAAG | CGCCTGACTGGTCCTTATATTC | 60 °C |
| lrgB | TATTGCCCGAGGATTAGCAC | CAAAGACAGGCACAACTGCTAC | 60 °C |
| lukD | GTACTTAAGGCAGCCGGAAAC | CGCCCCAATAAAACTGTGAG | 60 °C |
| sigB | TGATCGCGAACGAGAAATC | ATTGCCGTTCTCTGAAGTCG | 60 °C |
| capC | CATCCAGAGCGGAATAAAGC | CGGAAATACCCGCTAATGAC | 59.5 °C |
| isaA | TCCGACAAACACTGTTGACC | AATCCCCAAGCACCTAAACC | 59.5 °C |
| lytM | ACGGTGTCGACTATGCAATG | ATTGCCGCCACCATAGTTAC | 59.5 °C |
| saeR | CCAAGGGAACTCGTTTTACG | ACGCATAGGGACTTCGTGAC | 59.5 °C |
| fnbB | GAACATGGTCAAGCACAAGG | ACGCCATAATTACCGTGACC | 59 ºC |
| hla | TCTTGGAACCCGGTATATGG | AGCGAAGTCTGGTGAAAACC | 59 ºC |
| hlb | GTGCCAAAGCCGAATCTAAG | ATCAGCGCGTTTATATTGTCC | 59 ºC |
| hld | AAGGAAGGAGTGATTTCAATGG | TTTGTTCACTGTGTCGATAATCC | 59 ºC |
| psm a | TCAAAAGCTTAATCGAACAATTCAC | AATGGCCCCCTTCAAATAAG | 59 ºC |
| sarA | TTGCTTTGAGTTGTTATCAATGG | CAATACAGCGAATTCTTCAAAGC | 59 °C |
| cidA | CTTAGCCGGCAGTATTGTTG | GTTTGCACCGTCTTCTACCC | 58.5 °C |
| clfB | TTATGGTGGTGGAAGTGCTG | TGGACTTGGTTCTGGATCTG | 58.5 ºC |
| RNAIII | AAGCCATCCCAACTTAATAACC | GCACTGAGTCCAAGGAAACTAAC | 58.5 °C |
| rpoB | ACAACCACTTGGCGGTAAAG | ATGCTTCAAGTGCCCATACC | 60 °C (58.5, 59, 59.5) |

### Cytokine and Cytotoxicity Assays

CCL81 (Vero) and HT29 cells were cultivated in DMEM consisting of 10% fetal bovine serum and 0.1% antibiotic. Cells were cultured for 7 days at 37 °C in a carbogen (95% 02, 5% CO2) atmosphere, with the culture medium being changed every two to three days until cells were confluent. For CCL81 cell lines, approximately 2×10⁴ cells were seeded into new 96 well plates (Corning, USA) and allowed to form monolayers for 48 hours prior to cytotoxicity testing. For HT29 cells, approximately 2×10⁴ cells were seeded into new 24-well plates (Corning, USA) and allowed to differentiate for 33 days prior to treatment. After removing media, cells were washed twice with DPBS and equilibrated as previously described before adding bacteria (65,66). Exponential phase S. aureus ATCC 29213 cultures grown in TSB were standardized to OD₆₀₀ 1.0, centrifuged, washed with DPBS, and applied to appropriate test wells at a final concentration of 6.86×10⁷ CFU/well. After treatment, cells were centrifuged at 600 g for 5 minutes. Using HT-29 cells, the human pro-inflammatory TNF-α, the dual functioning IL-6 and the anti-inflammatory IL-10 ELISAs were performed using Quantikine® Colorimetric Sandwich ELISA Kits (R&D Systems, USA) according to the manufacturer's protocol. Cytotoxicity was determined using a lactate dehydrogenase (LDH) assay kit according to the manufacturer's protocol (Abcam, UK). All treatments were performed in triplicate and assayed in duplicate.

### Statistical Analysis

Results are presented as mean±standard error of the mean (SEM) unless noted otherwise. Statistical analyses were performed using GraphPad Prism 9.5.0 (San Diego, CA). Differences where p<0.05 were considered significant. Differences in biofilm inhibition were analyzed using a 2-tailed unpaired t-test. Sedimentation assays were analyzed using two-way ANOVA followed by Tukey's multiple comparisons. Antibiotic susceptibility, Vero cell cytotoxicity, and competition results were analyzed using one-way ANOVA, followed by Tukey's multiple comparisons.

### Example 1: B. subtilis 6D1 Inhibits S. aureus Biofilm Growth but not Planktonic Growth

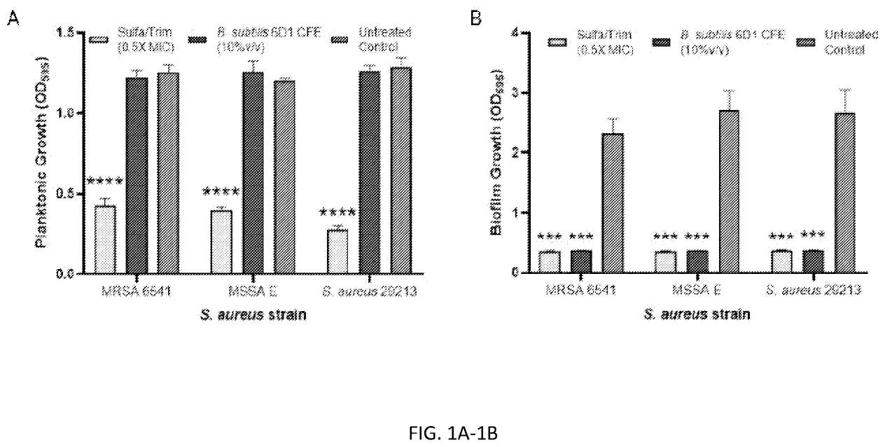

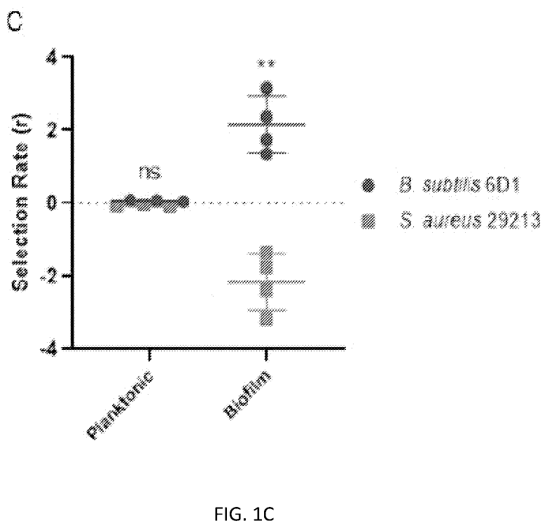

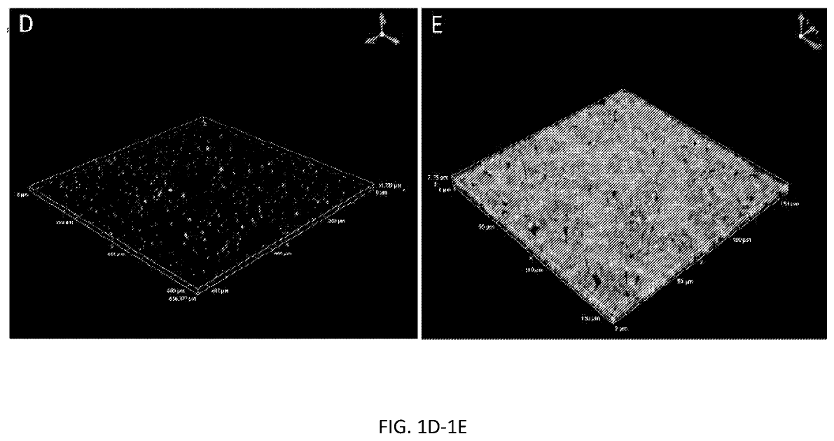

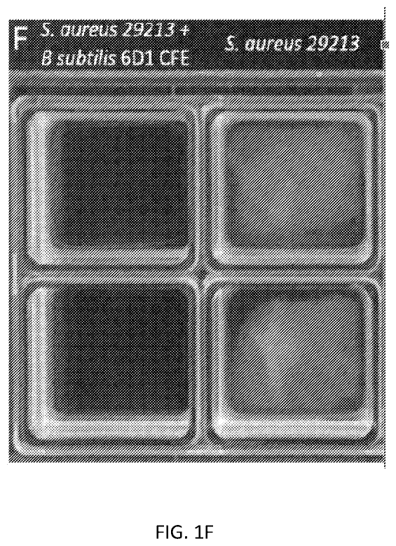

Compared to 0.5×MIC sulfamethoxazole/trimethoprim, B. subtilis 6D1 CFE applied at 10% v/v significantly reduced both MRSA and MSSA biofilm formation (FIG. 1B) without inhibiting planktonic growth (FIG. 1A). Confocal image analysis confirmed antibiofilm activity of 10% v/v B. subtilis 6D1 CFE, reducing mean Syto9 intensity 77%, from 235.64±SD 236.36 in an untreated control to 55.18±SD 61.72 under treated conditions (FIGS. 1D-1E). Macroscopic observation of S. aureus ATCC 29213 biofilms grown for 24 hours and washed prior to Syto9 staining corroborated microscopic imaging (FIG. 1F). Competition experiments in planktonic and biofilm environments also revealed B. subtilis 6D1 outcompetes S. aureus ATCC 29213 in a biofilm but not in a planktonic environment (FIG. 1C), suggesting antibiofilm activity is maintained during cell to cell interactions and not simply an artifact of monoculture CFE preparations. Taken together these results demonstrate compounds produced by B. subtilis 6D1 are capable of inhibiting biofilm formation in S. aureus.

### Example 2: B. subtilis 6D1 Cell Free Extracts Reduce S. aureus ATCC 29213 Biofilm Growth, Disassemble Mature Biofilm, and Improve Biofilm Inhibition when Applied in Conjunction with Low Doses of Antibiotics

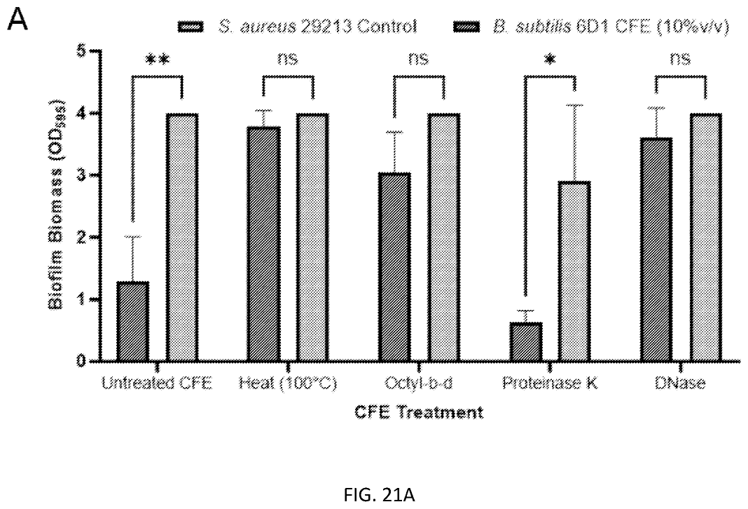

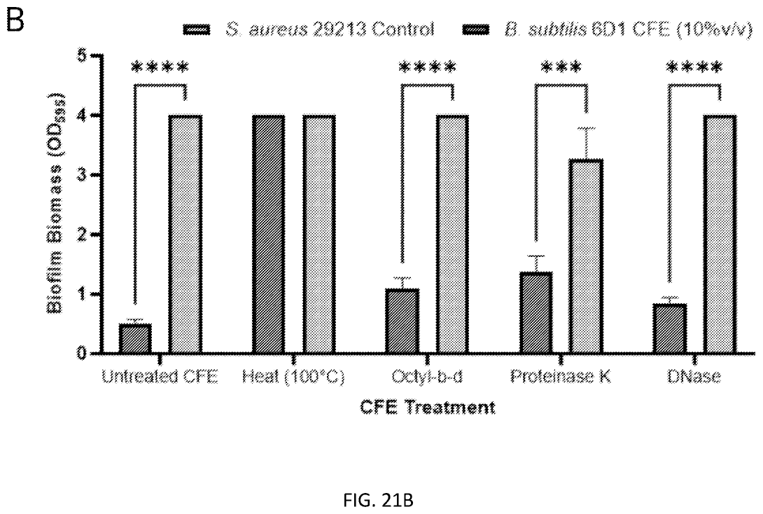

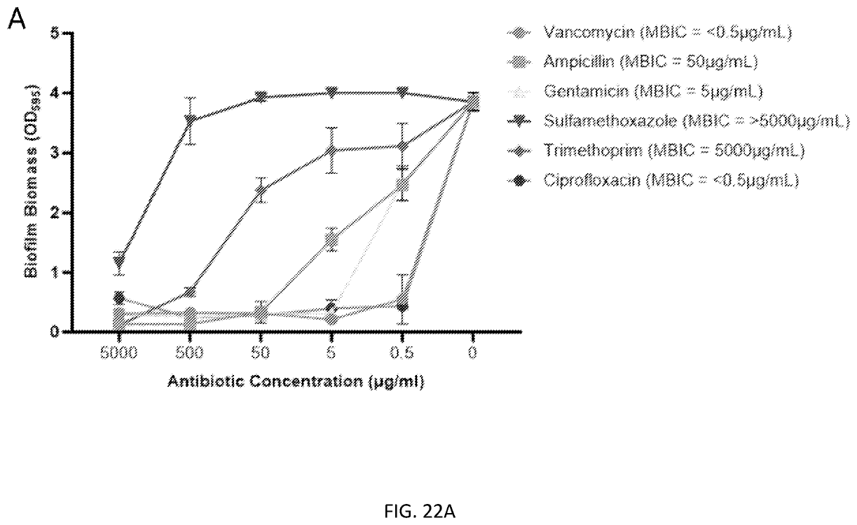

To investigate which bioactive molecules were driving the antibiofilm activity of B. subtilis 6D1 CFE, CFEs were exposed to different molecular inhibitors and re-evaluated for their ability to prevent S. aureus biofilm growth and disassemble mature S. aureus biofilm. Briefly, B. subtilis 6D1 CFE was separated into four different 200 μL aliquots and treated with either 50 μg/mL proteinase K (Thermo Scientific, USA), 2 units of DNaseI (Thermo Scientific, USA), or 10 mM Octyl-β-D-Glucopyranoside (Sigma-Aldrich, USA), for 30 minutes at 37 °C. (Ahn et al. 2018). A fourth heat treatment exposed CFE to 100 °C for 10 minutes. The antibiofilm activity of CFE was lost following heat exposure (FIGS. 21A-B).

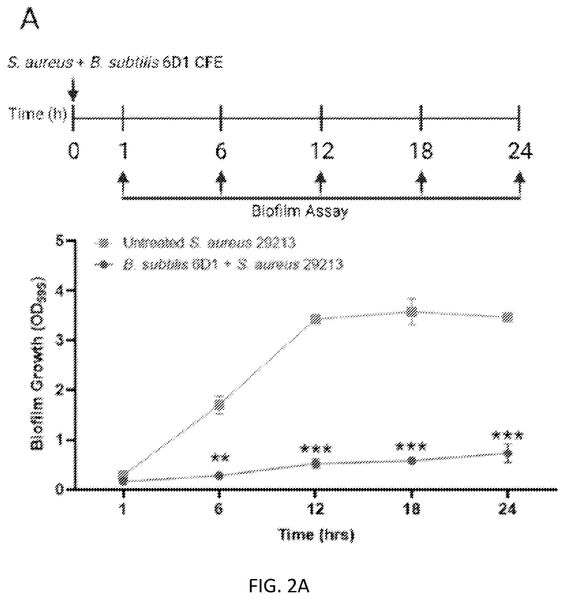

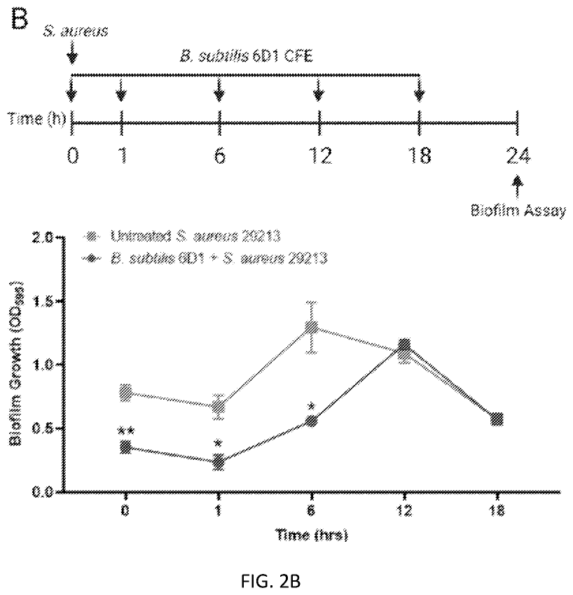

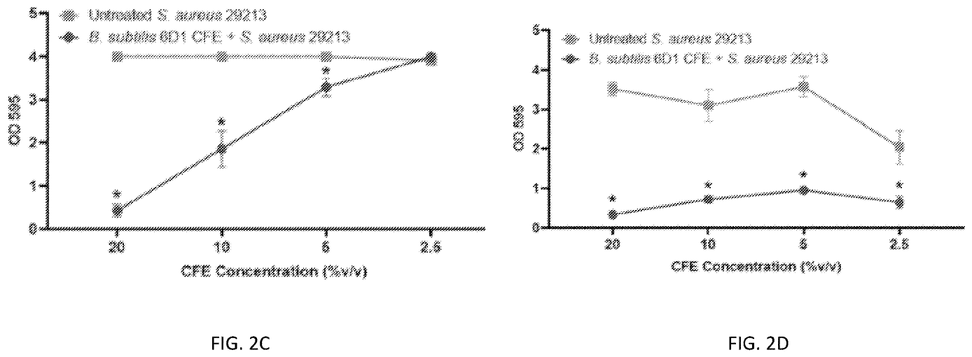

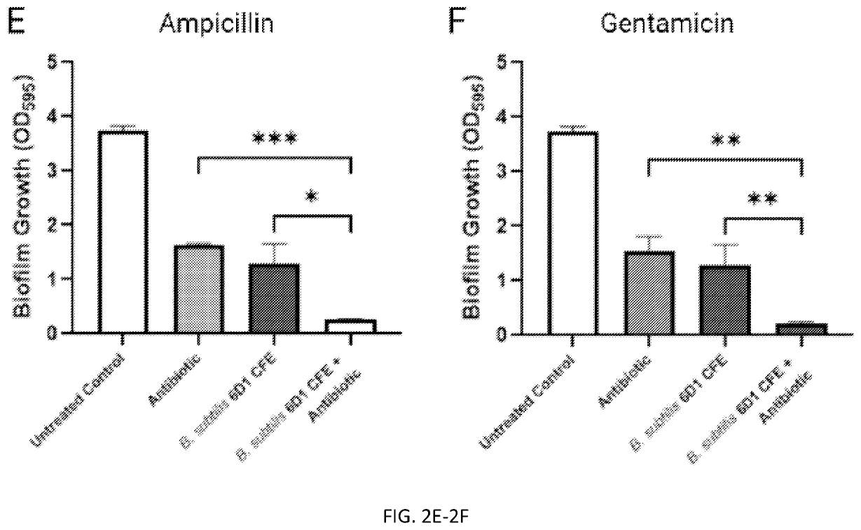

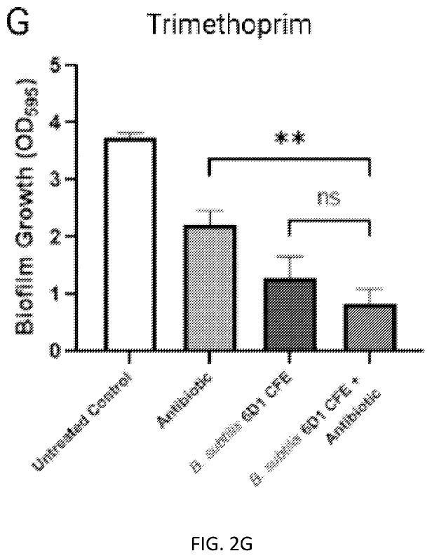

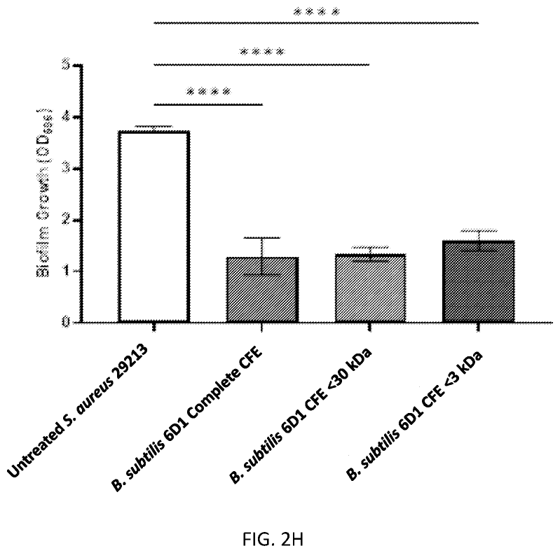

To assess which stages of the S. aureus biofilm life cycle (67) were impacted by B. subtilis 6D1, 10% v/v CFE was applied at various timepoints throughout biofilm growth. If applied within the first six hours, B. subtilis 6D1 CFE reduced biofilm growth (FIG. 2B), and biofilm formation did not recover after initial CFE exposure (FIG. 2A). Taken together, these results suggest B. subtilis 6D1 produces soluble elements that inhibit biofilm adherence and early maturation-processes typically under the control of the Agr QS system (68). Furthermore, addition of B. subtilis 6D1 CFE reduced S. aureus ATCC 29213 biofilm growth (FIG. 2C) and disrupted mature biofilm (FIG. 2D) in a concentration dependent manner, indicating a positive correlation between active compound abundance and antibiofilm activity. Subsequent size fractionation analyses revealed antibiofilm activity was maintained in CFE fractions <3 kDa (FIG. 2H), suggesting small peptides may be driving this effect.

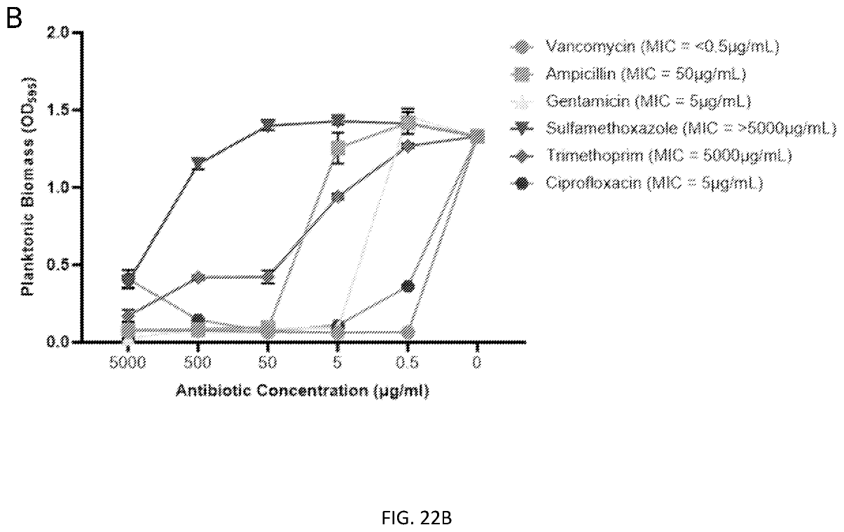

These findings were then used to ask whether application of B. subtilis 6D1 CFE improved biofilm inhibition if applied in conjunction with low doses of antibiotics. Ampicillin, Gentamicin, and Trimethoprim were selected for both their clinical relevance in treating Staphylococcal infections and their different antimicrobial mechanisms-ampicillin interferes with cell wall synthesis, gentamicin inhibits protein translation, and trimethoprim prevents DNA synthesis. To quantify synergistic, additive, indifferent, or antagonistic effects brought on by the addition of B. subtilis 6D1 CFE, minimum biofilm inhibition concentrations (MBIC) and minimum inhibitory concentrations (MIC) were acquired for each antibiotic to identify the lowest permissible concentration that would allow S. aureus ATCC 29213 planktonic and biofilm growth (FIGS. 22A-B) Application of 10% v/v B. subtilis 6D1 CFE in conjunction with 0.5 μg/ml Ampicillin (FICI=0.34±0.08) and 0.5 μg/ml Gentamicin (FICI=0.28±0.04) synergistically reduced S. aureus ATCC 29213 biofilm formation and possessed an additive inhibitory effect when applied in conjunction with 0.5 μg/ml Trimethoprim (FICI=0.87±0.08) (FIGS. 2E-G).

### Example 3: B. subtilis 6D1 Modulates Gene Expression Associated with S. aureus Quorum Sensing

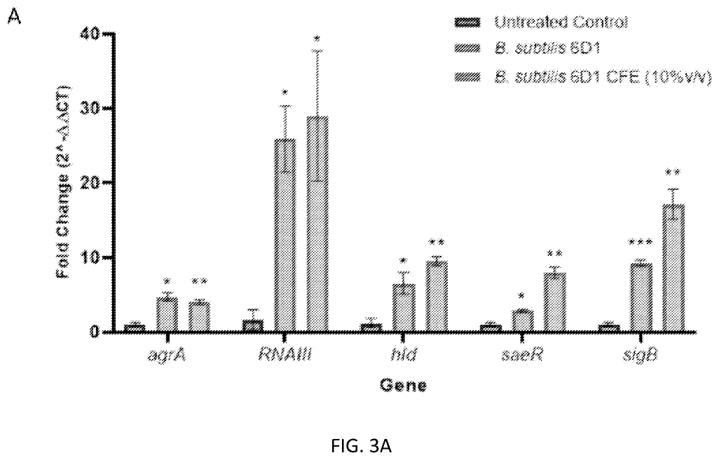

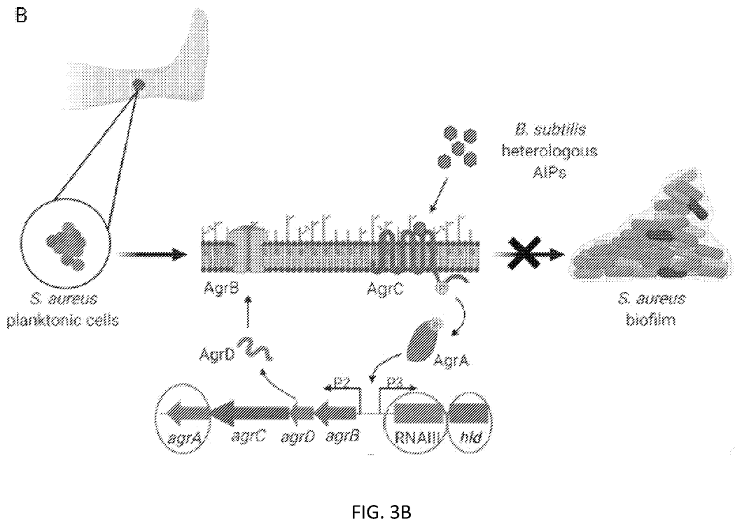

RT-qPCR gene expression experiments targeting an array of biofilm and QS related genes (Table 8) revealed both B. subtilis 6D1 and its associated cell free extract exhibited antibiofilm activity through Agr QS interference of S. aureus (FIG. 3A). Specifically, these data demonstrated that both 10% v/v CFE and B. subtilis 6D1 cells applied in a 1:1 ratio with S. aureus ATCC 29213 increased expression of agrA, RNAIII, and hld genes. B. subtilis 6D1 increased agrA, RNAIII, and hld expression 5-fold, 26-fold, and 6-fold, respectively. B. subtilis 6D1 CFE (10% v/v) increased expression of these same genes 4-fold, 29-fold, and 9-fold, respectively. AgrA, RNAIII, and hld are all located in the S. aureus Agr operon and when upregulated, transition cells from a biofilm lifestyle to a planktonic lifestyle (FIG. 3B). Other genes also upregulated in response to B. subtilis 6D1 exposure included the global stress response regulator, sigB, and saeR, the response regulator of a two component signal transduction system that regulates the expression of numerous virulence factors in S. aureus, including surface-bound and secreted proteins (69). Compared to the 1:1 B. subtilis 6D1 cell treatment, B. subtilis 6D1 CFE elicited a 9.2-fold and 6.3-fold stronger expression response for both sigB and saeR, respectively.

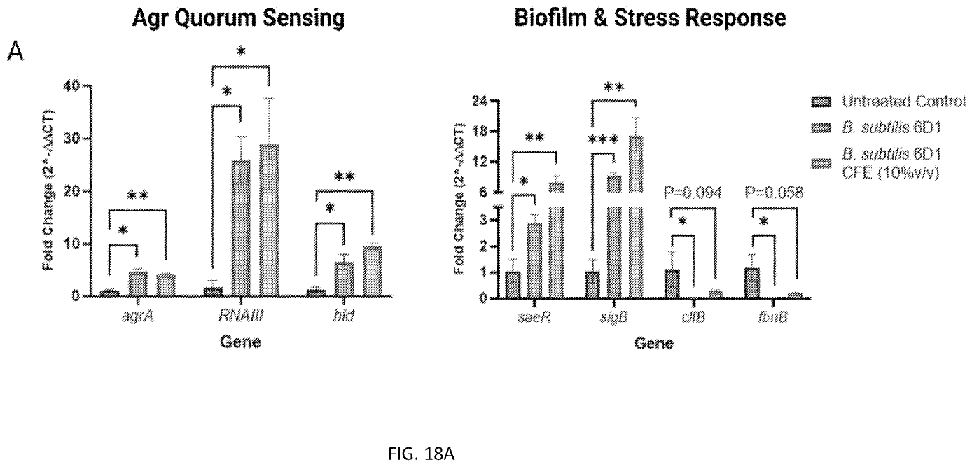

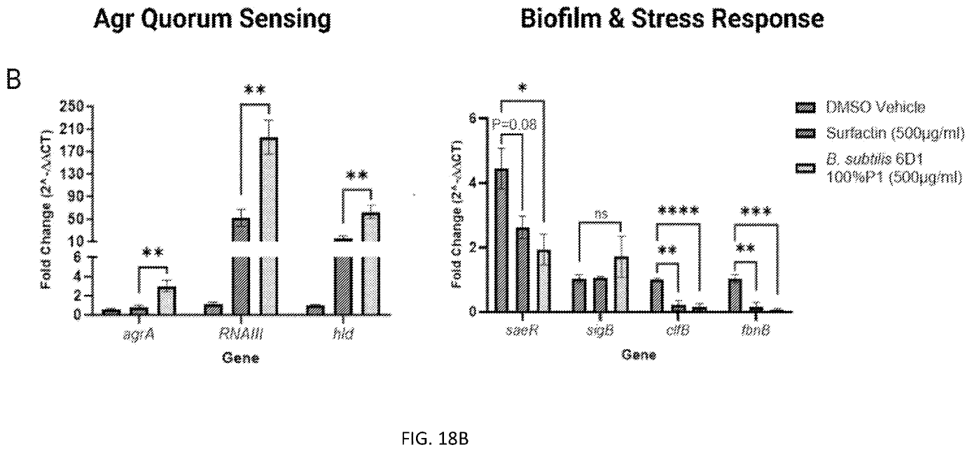

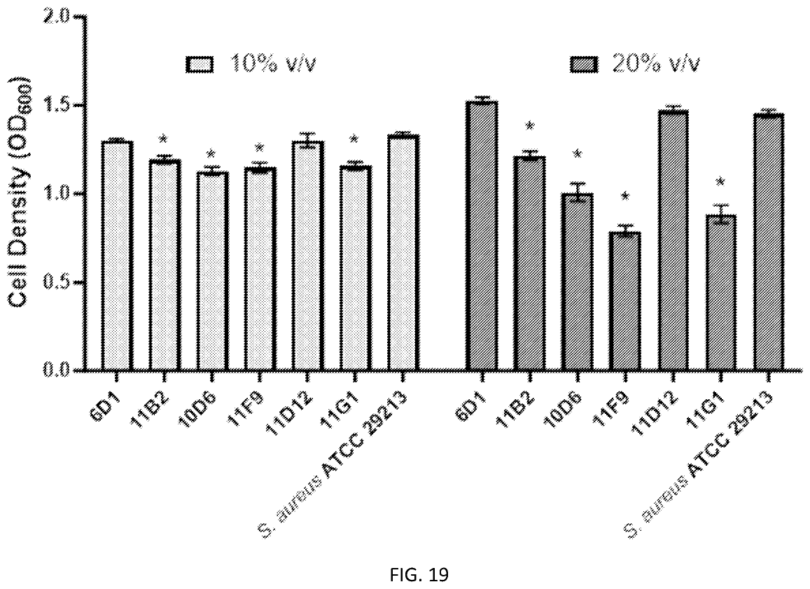

Based on our previous data showing an increase in antibiofilm activity of fraction 100% P1 compared to commercial surfactin, follow-up gene expression studies were conducted to determine whether these peptide mixtures elicited different gene expression responses in S. aureus. In line with our previous observations, gene expression analysis confirmed 100% P1 applied at 500 μg/mL increased Agr-related gene expression more than commercial surfactin applied at the same concentration (FIG. 18B Left). Despite these differences, both peptide mixtures elicited a stronger

Agr-mediated gene expression response than either B. subtilis 6D1 or the associated CFE. Compared to the CFE treatment, application of the purified 100% P1 fraction increased RNAIII and hld expression 166-fold and 53-fold, respectively. Interestingly, neither surfactin nor the 100% P1 fraction increased sigB or saeR expression that was observed in the B. subtilis 6D1 or associated CFE treatments (FIG. 18B Right). These data suggest different compounds present in the CFE are prompting the expression of multiple regulatory pathways in S. aureus including those responsible for Agr quorum sensing.

### Example 4: B. subtilis 6D1 Genome Possesses Multiple Unique Biosynthetic Gene Clusters

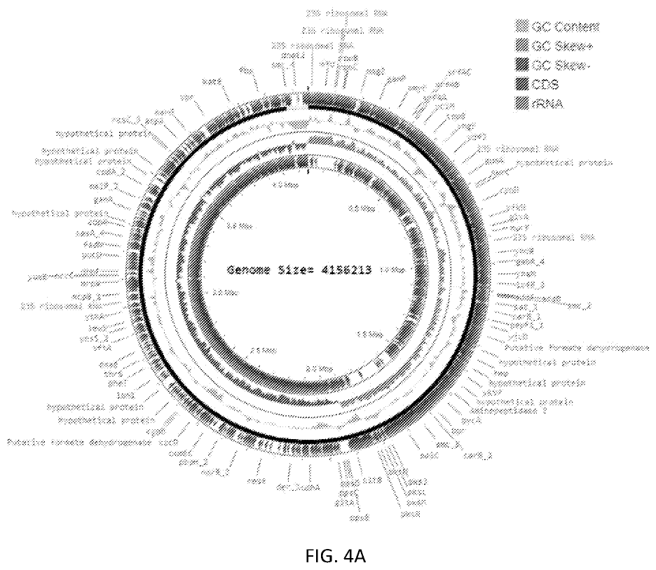

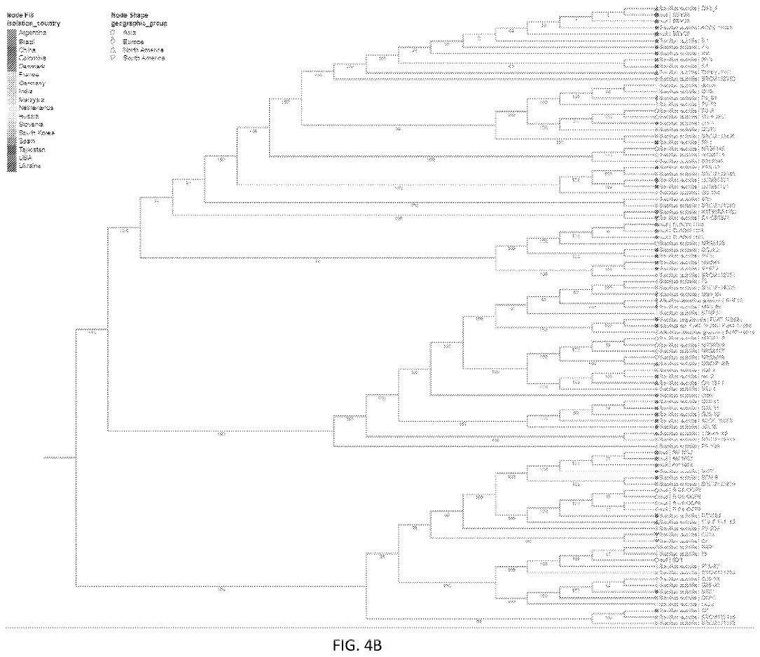

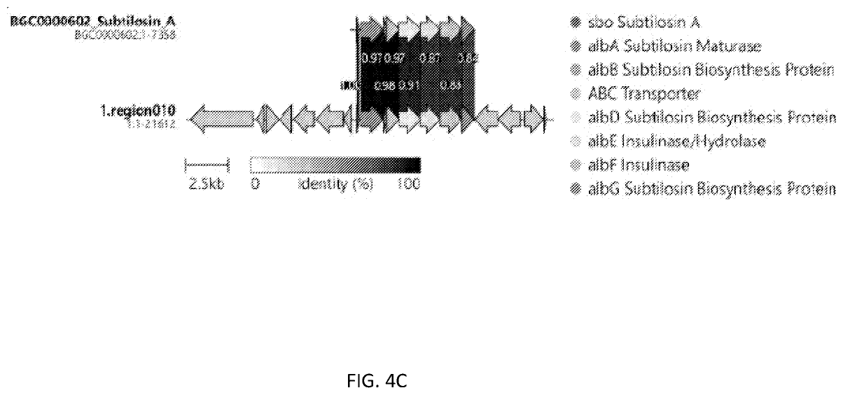

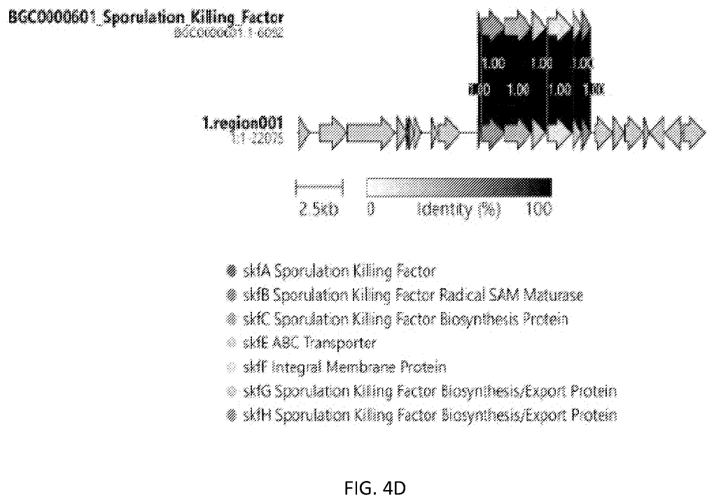

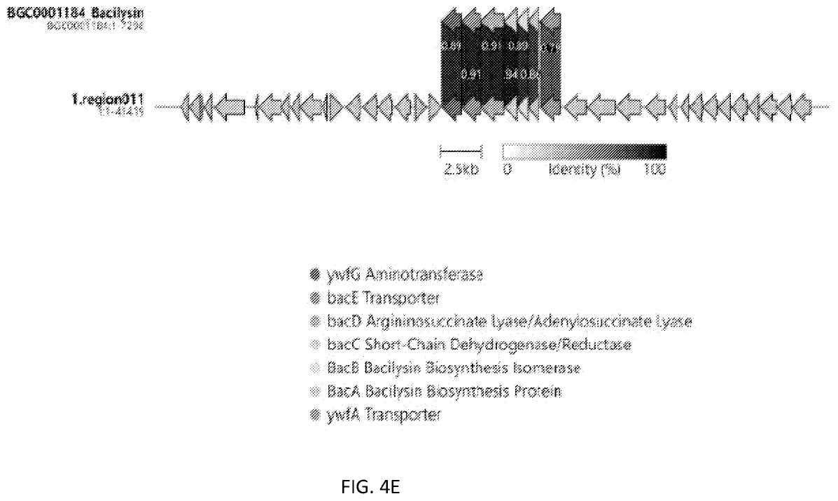

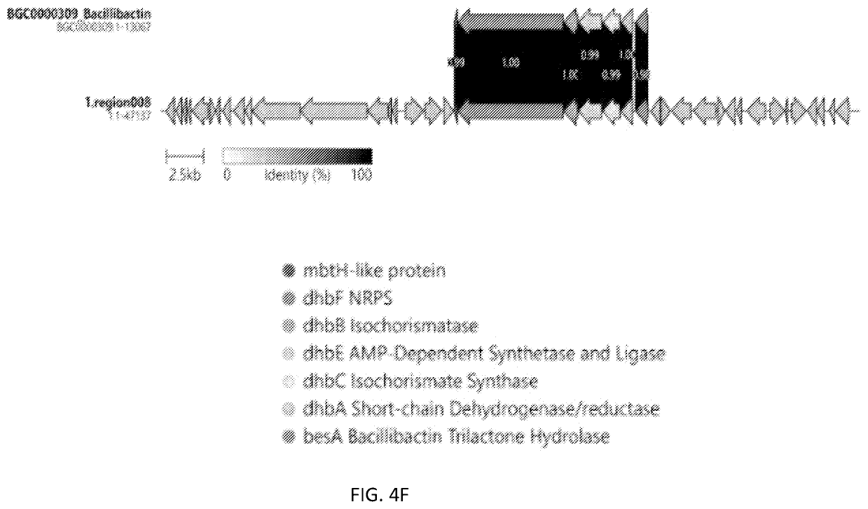

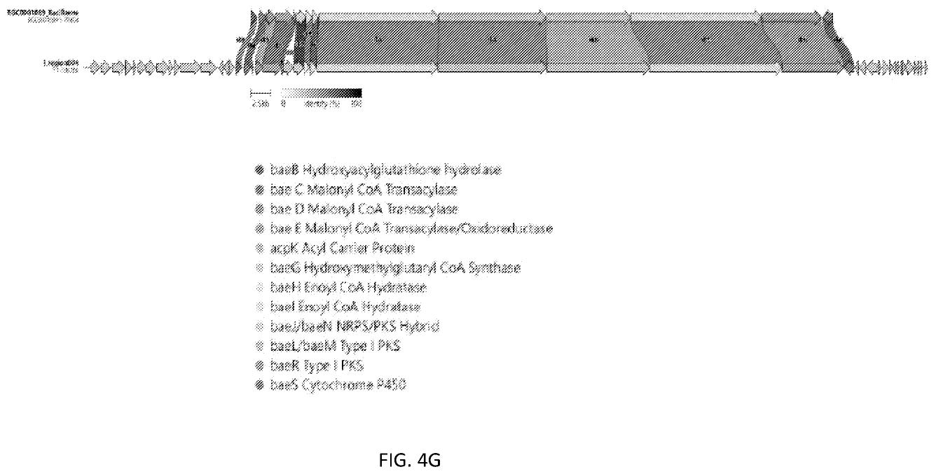

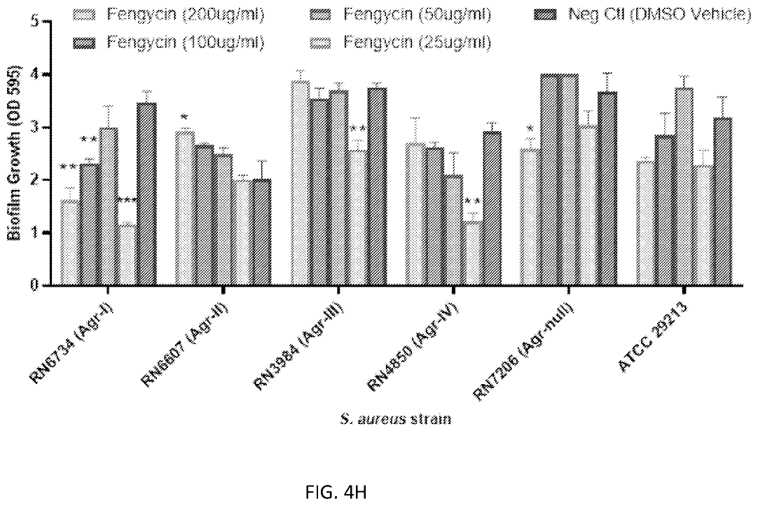

High-throughput screening of presumptive Bacillus strains revealed B. subtilis 6D1 exhibited the strongest antibiofilm activity against S. aureus compared to the 16 most potent 154 environmentally sourced strains (FIG. 17A). Whole genome sequencing of B. subtilis 6D1 was performed to search for biosynthetic gene clusters (BGCs) that might explain this strain's antibiofilm activity. Genomic assembly yielded two contigs, the chromosome and an 84 kB plasmid similar to pBS32, a large ancestral plasmid rarely found in environmental isolates (70,71) (FIG. 4A, 17B). The largest contig and N50 were 4,072,344 bp.

The entire genome contained 4,156,213 base pairs with 43.65% GC content (Gene Bank Accession #). A total of 4,309 protein coding genes (CDS), 88 transfer RNA (tRNA) genes, and 30 ribosomal RNA (rRNA) genes were predicted. Of the 4,309 CDS predicted, 708 were hypothetical proteins, and 3,601 proteins had functional assignments—these included 1,055 proteins with Enzyme Commission (EC) numbers, 881 with Gene Ontology (GO) assignments, and 776 proteins that were mapped to KEGG pathways. Sixteen antimicrobial resistance genes were identified only in the chromosome and were not flanked by any predicted mobile genetic elements (FIG. 17B). Taxonomic identification and phylogenetic analysis confirmed this genome was a member of the B. subtilis group and was most similar to B. subtilis 75 (Gene Bank Accession #CP045825), and B. subtilis PTA-271 (Gene Bank Accession #JACERQ010000010.1), strains sourced from the rhizosphere of high performing plant species capable of reducing plant pathogen colonization (FIG. 4B).

Bioinformatics analysis (AntiSmash v6.0) identified seven biosynthetic gene clusters that were subsequently mapped to reference BGCs identified in the undomesticated B. subtilis NCIB 3610 strain (GenBank Accession #257 ASM205596v1). Of these seven predicted BGCs, five were found to possess 100% similarity with reference clusters: subtilosin A (FIG. 4C), sporulation killing factor (FIG. 4D), bacilysin (FIG. 4E), bacillibactin (FIG. 4F), and bacillaene (FIG. 4G). Interestingly, two BGCs showed 78% similarity to surfactin and 93% similarity to fengycin, non-ribosomal peptides (NRP) with previously demonstrated quorum sensing interference and antibiofilm capabilities (72,73). However, subsequent biofilm inhibition experiments using commercial fengycin obtained from B. subtilis revealed this compound increased biofilm formation in multiple Agr backgrounds (FIG. 4H). Upon closer examination, it was determined that the initially identified fengycin cluster was instead, plipastatin, a very similar lipopeptide albeit with a slightly different structural moiety (74) found more frequently in B. subtilis subsp. subtilis strains than in other members of the B. subtilis group (38). Furthermore, compared to the plipastatin reference BGC, the B. subtilis 6D1 BGC has lost both ppsA and ppsB non-ribosomal peptide synthetase (NRPS) genes (FIG. 4I, top). Compared to the surfactin reference BGC, srfAA and srfAB NRPS genes identified in B. subtilis 6D1 shared 75% and 77% identity, respectively (FIG. 4I, bottom). B. subtilis 6D1 also harbors an additional ABC transporter downstream of yciC not identified in the reference BGC. Taken together, B. subtilis 6D1 harbors unique biosynthetic gene clusters that closely resemble surfactin and plipastatin BGCs-NRPs capable of inhibiting S. aureus biofilm and interfering with Agr quorum sensing. Therefore, ethyl acetate extractions were performed to determine if these lipopeptides are produced, and whether they contribute towards the antibiofilm activity observed.

Phylogenomic analysis revealed B. subtilis 6D1 shares >95% identity with a recently evolved group of B. subtilis genomes (FIG. 17C); however, this strain encodes an additional 156 singleton genes that were not present in these closely related strains (FIG. 17D). Many of the singleton genes were of unknown function, but genes encoding a glycosyl hydrolase were identified. This class of enzymes can hydrolyze the glycosidic bonds between sugars, such as those found within the exopolysaccharides of biofilm matrices, and have been shown to reduce biofilm biomass by weakening the matrix and inducing bacterial dispersal (178, 179). Interestingly, glycosyl hydrolases can inhibit and degrade Staphylococcal and Pseudomonal biofilms and improve clearance of these pathogens when applied in conjunction with antibiotics (179). The second largest group of singleton genes identified were predicted to be of viral origin, including prophage genes from the family Siphoviridae. Genome mapping further confirmed the presence and location of a 25 kB and 27 kB prophage region predicted to belong to the Siphoviridae and Myoviridae family, respectively (FIG. 17B). The B. subtilis 6D1 strain appeared to maintain many ancestral B. subtilis genes (FIG. 17D), including a PBS32-like plasmid (FIG. 1B); however, our results suggest this strain may have recently acquired foreign genetic elements, possibly from phage, that increased its ability to degrade pathogenic biofilms.

### Example 5: B. subtilis 6D1-CFE Derived Peptides Inhibit Biofilm Growth in all Agr Backgrounds and an Agr Null Derivative

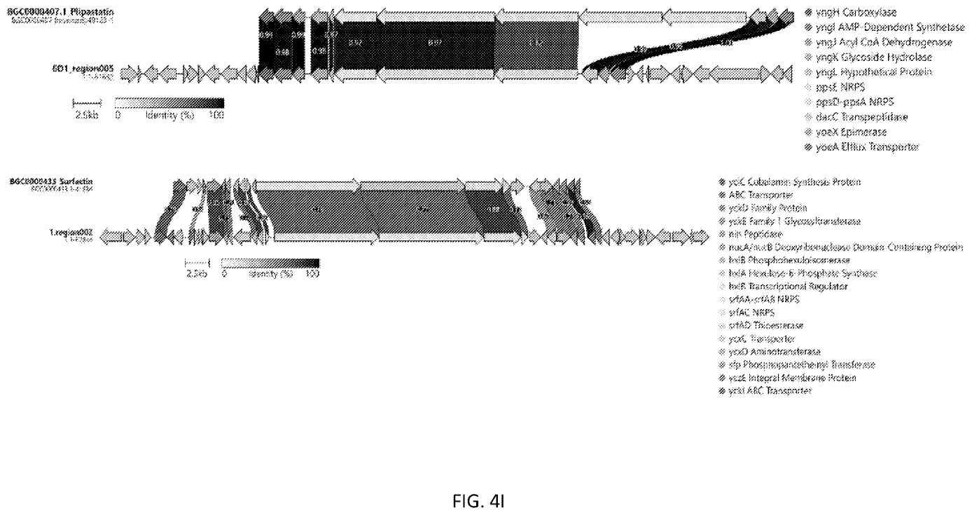

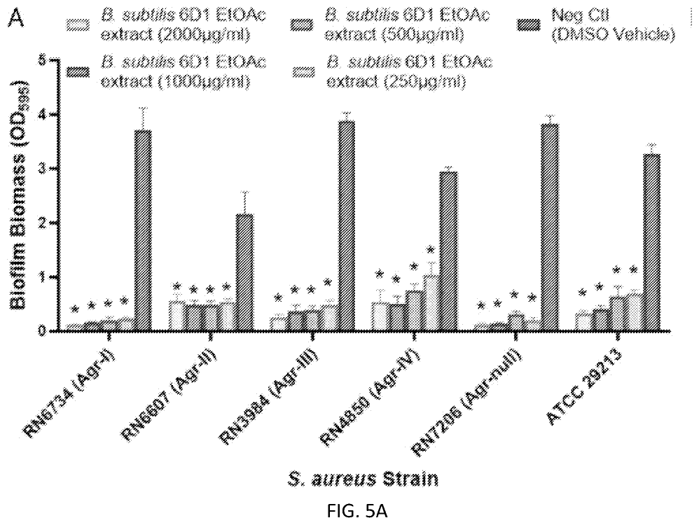

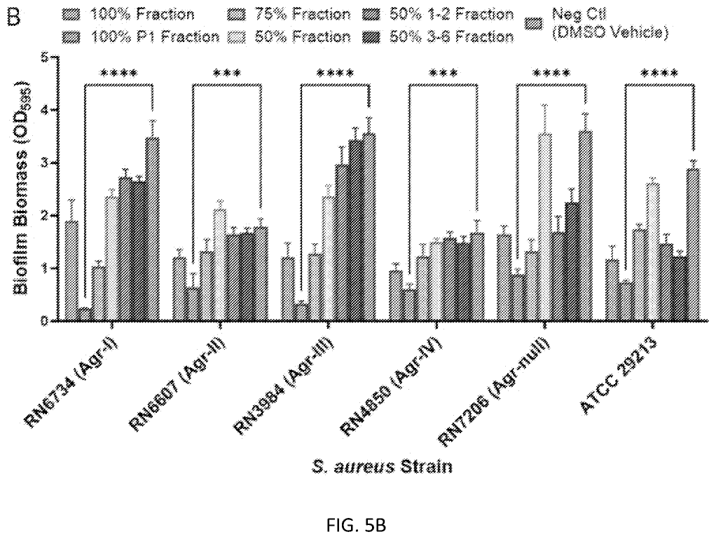

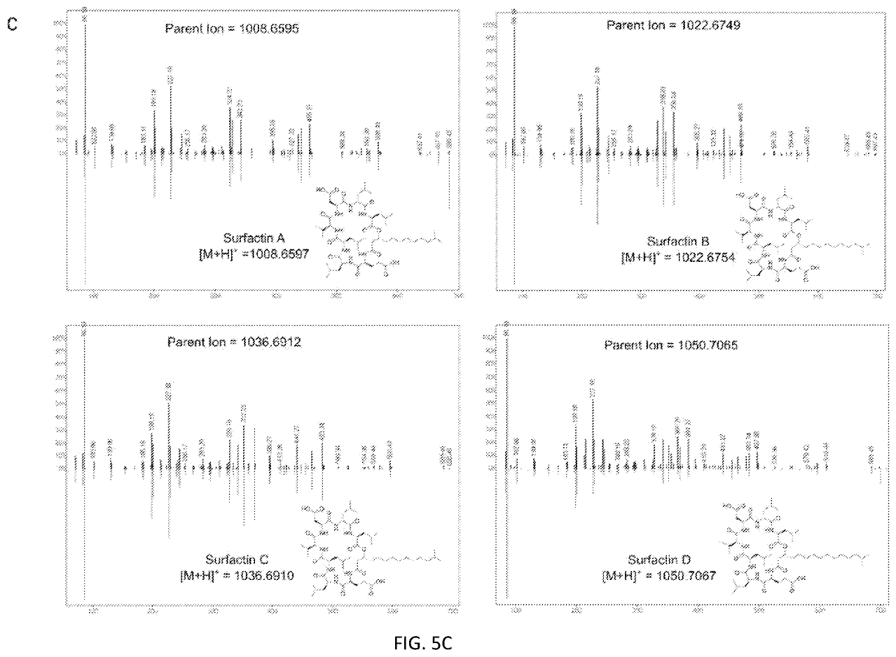

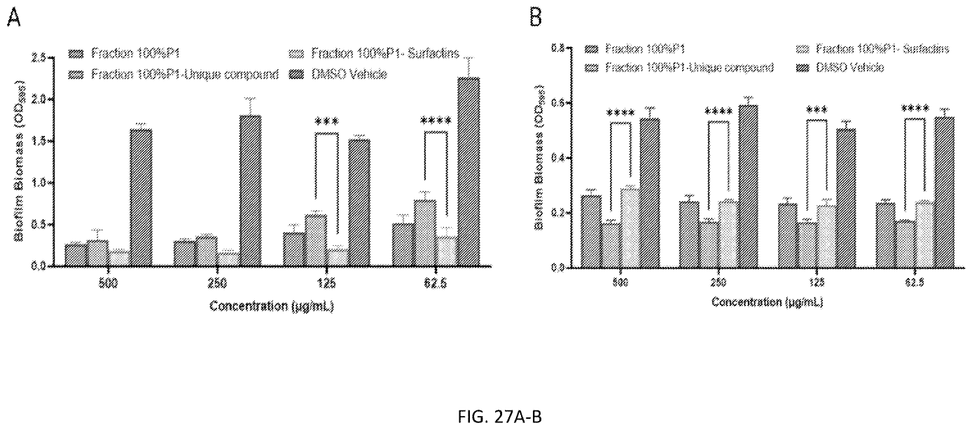

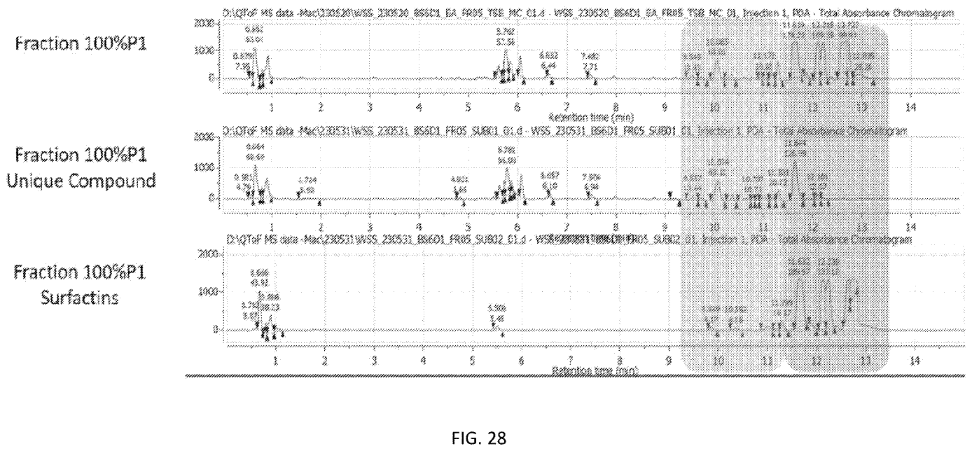

S. aureus strains can harbor one of four distinct Agr QS systems, and though each Agr system has been associated with a different biological consequence, Agr QS system in each group is responsible for regulating virulence factor gene expression and facilitating biofilm formation and dispersal. To assess whether B. subtilis 6D1 CFE lipopeptides were driving S. aureus antibiofilm activity, a crude ethyl acetate extract resuspended in DMSO was also assessed for its antibiofilm activity against S. aureus strains possessing different Agr backgrounds. Not only did this extract maintain dose-dependent activity, the ability of the extract to inhibit biofilm formation in an Agr null background suggests that it was Bacillus lipopeptides, not self-stimulated production of AgrD, that were binding to AgrC and stimulating the phosphorylation of AgrA and the subsequent expression of genes within the Agr operon (FIG. 5A). B. subtilis 6D1 ethyl acetate extracts were separated further by flash chromatography using a stepwise gradient of water and methanol to obtain six distinct fractions with unique LC-MS/MS spectra. After drying, concentrated fractions were dissolved in DMSO and again evaluated for their ability to inhibit S. aureus biofilm formation. Compared to DMSO vehicle controls, fraction 100% P1 harbored the broadest and strongest antibiofilm activity among the six fractions (FIG. 5B). Though other fractions harbored antibiofilm activity, namely 100% and 75% fractions, 100% P1 yielded the most consistent antibiofilm activity across all Agr backgrounds. Feature based molecular networking through GNPS confirmed the presence of surfactin A-D in fraction 100% P1 (FIG. 5C). Surfactins A-D were identified by comparison of their LC-MS/MS fragmentation patterns and molecular formulae, based on accurate mass, to spectra in the GNPS public spectral database. A smaller sub-network with no known matches to GNPS database spectra was also identified in this fraction (FIG. 5D), and subsequent separation of this unique compound, Compound E, from the surfactins revealed it, too, possessed potent antibiofilm activity, including an increased disruption of mature biofilm as compared to the fraction containing surfactins (FIGS. 27A-B, 28). Both B. subtilis NCIB 3610 and another environmental B. subtilis strain, 9B5, harboring moderate antibiofilm activity were analyzed via LC-MS/MS alongside B. subtilis 6D1, but neither strain possessed this unknown sub-network. Collectively, lipopeptides produced by B. subtilis 6D1, namely surfactin A-D, and the unique compound, inhibit biofilm growth in all four S. aureus Agr backgrounds and in an Agr null strain incapable of producing its own autoinducing peptide, AgrD.

### Example 6: B. subtilis 6D1 100% P1 CFE Fraction Exhibits Unique Antibiofilm Activity Compared to HPLC Grade Surfactin Obtained from B. subtilis

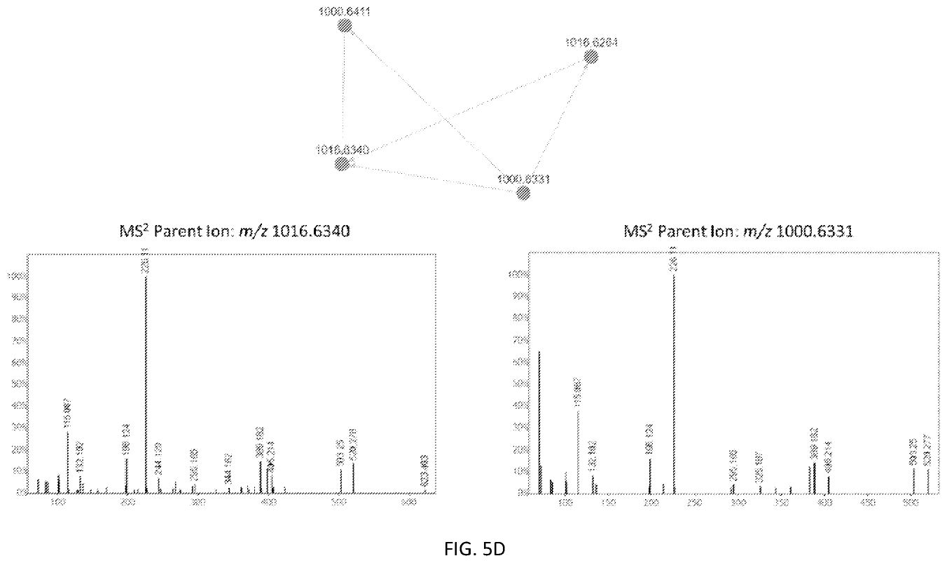

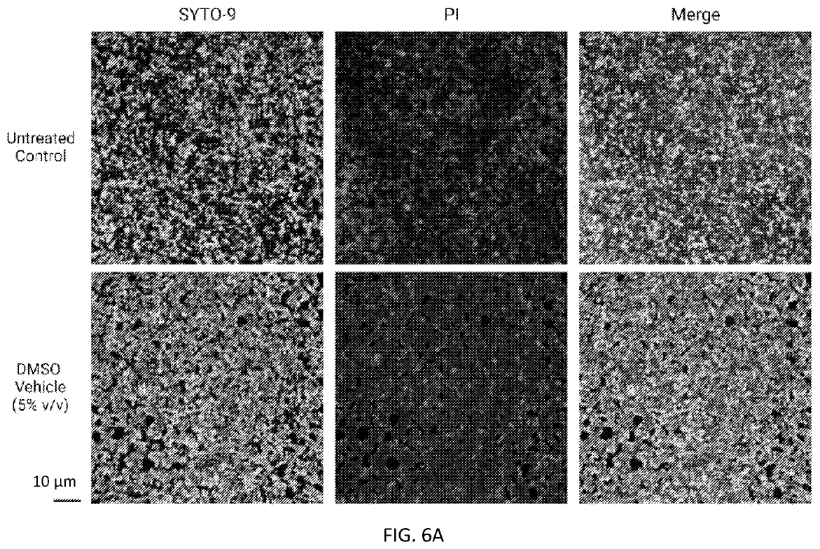

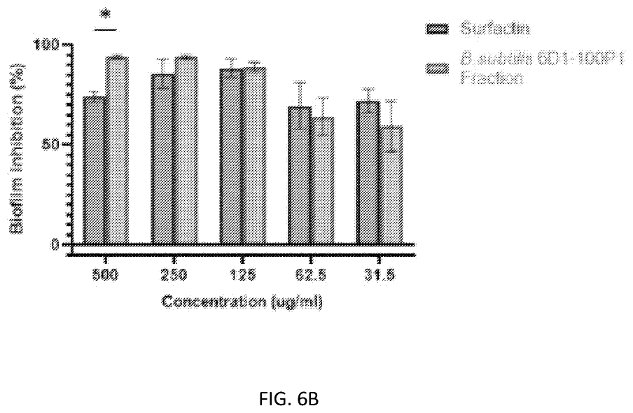

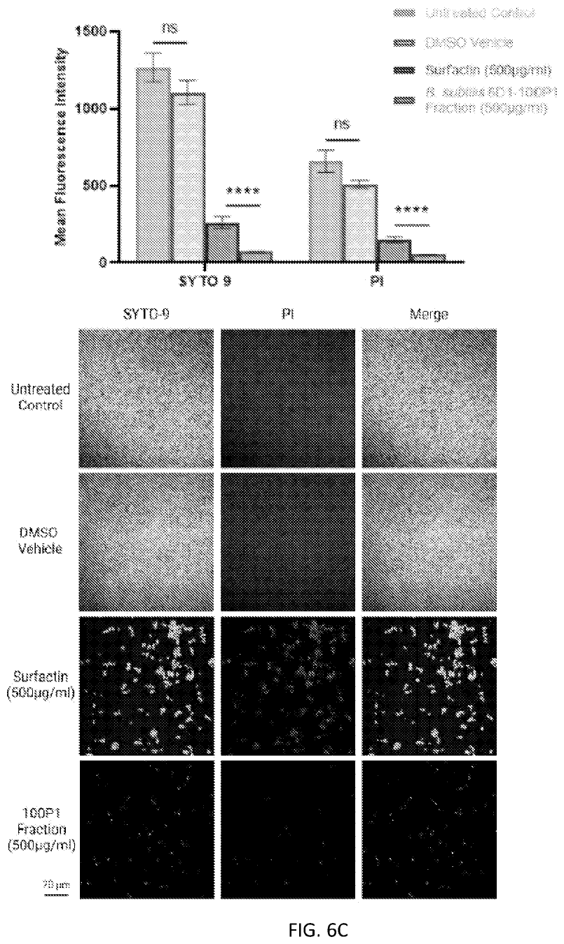

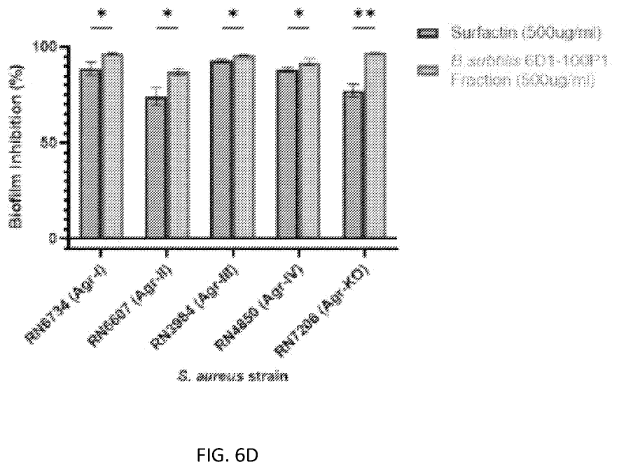

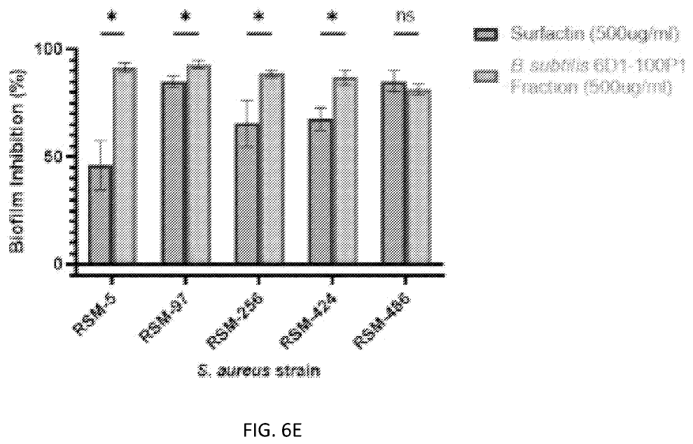

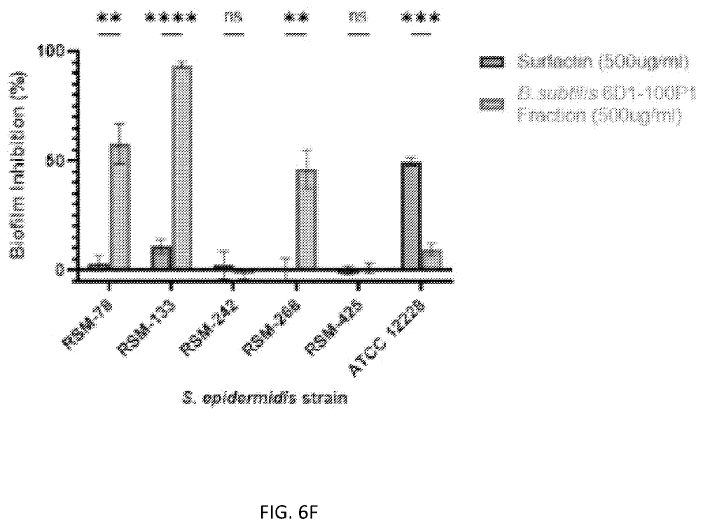

A series of biofilm inhibition experiments were performed comparing the 100% P1 fraction and commercial HPLC grade surfactin produced by B. subtilis (Sigma-Aldrich, USA). Both 100% P1 and commercially obtained surfactin were standardized in DMSO to 10 mg/ml working stock concentrations to ensure that both identical DMSO volumes and metabolite concentrations were applied to test conditions. Additional DMSO titrations confirmed volumes used to deliver these peptides did not increase cell death compared to the untreated biofilm controls (FIG. 6A). Preliminary titration experiments revealed that at 500 μg/ml, 100% P1 significantly increased S. aureus biofilm inhibition (94%) compared to commercial surfactin alone (73%) (FIG. 6B). Confocal image analysis confirmed these findings, reducing SYTO9 and propidium iodide (PI) mean fluorescence intensity from 263.3±34.3 and 152.6±17.9 in surfactin treated wells to 74.6±0.7 and 53.9±0.5 in 100% P1 treated wells (FIG. 6C). Based on these observations, 500 μg/ml concentrations were used in all ensuing experiments. At this concentration, B. subtilis 6D1 fraction 100% P1 possessed stronger antibiofilm activity against all Agr background strains with biofilm percent inhibition ranging from 88-97% while biofilm inhibition ranged from 74-92% for surfactin alone (FIG. 6D). This trend persisted when evaluating clinical S. aureus isolates. B. subtilis 6D1 fraction 100% P1 exhibited biofilm inhibition ranging from 82-93% while surfactin biofilm inhibition was much less consistent, ranging from 46-85% in the same isolates (FIG. 6E). Further separation of fraction 100% P1 revealed that both the surfactin mixture and the unique compound found in this fraction exhibited strong biofilm inhibition and disruption activity (FIG. 27A-B). Interestingly, differences between 100% P1 and surfactin were more apparent when evaluating biofilm inhibition activity against S. epidermidis, another species with a functioning, albeit slightly different Agr QS system that can also form biofilm on indwelling medical devices (75,76). Fraction 100% P1 successfully inhibited ⅗ clinical S. epidermidis isolates, however commercial surfactin was unable to inhibit any of the five clinical strains above 10% (FIG. 6F). Conversely, surfactin only inhibited biofilm formation of S. epidermidis ATCC 12228, while fraction 100% P1 did not have this effect. Based on this data, we conclude that at 500 μg/ml concentrations, B. subtilis 6D1 fraction 100% P1 exhibits stronger antibiofilm activity than commercial surfactin against S. aureus strains.

### Example 7: B. subtilis 6D1 CFE Reduces S. aureus ATCC 29213 Virulence in a Human Intestinal Cell Line

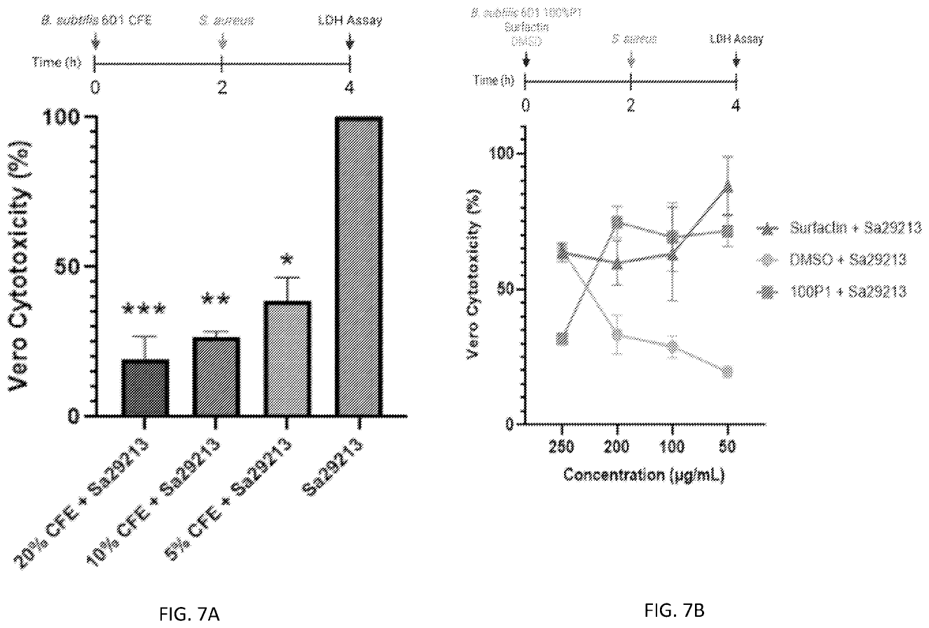

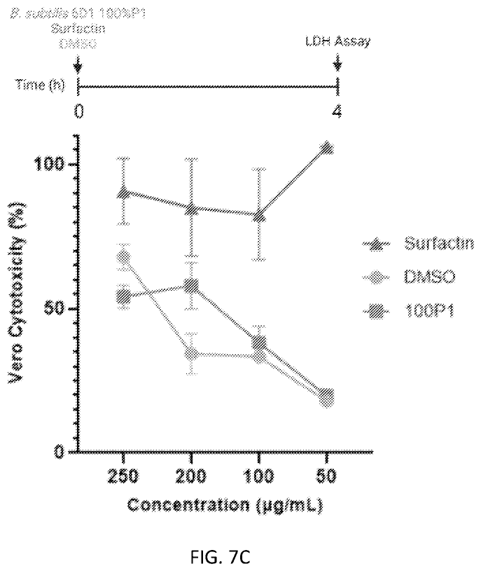

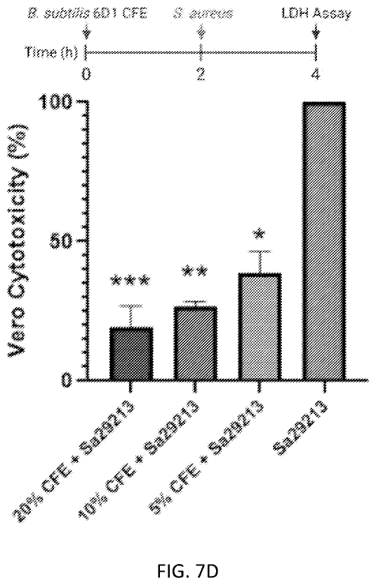

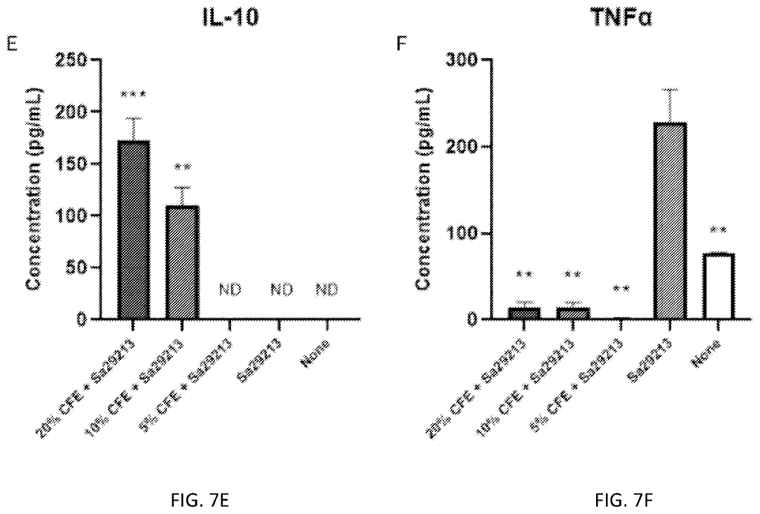

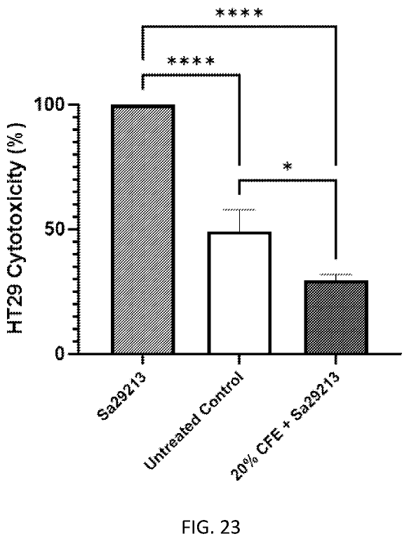

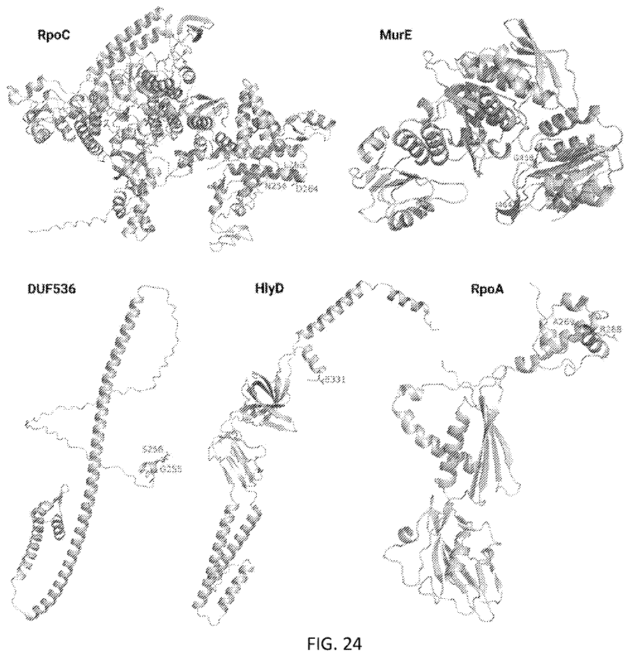

Both Vero (CCL81) and HT29 cell lines were used to assess the cytotoxic and ameliorative effects of B. subtilis 6D1 CFE, fraction 100% P1, and commercial surfactin in the presence and absence of a S. aureus challenge. Both 100% P1 and commercially obtained surfactin were standardized in DMSO to 10 mg/ml working stock concentrations to ensure that both identical DMSO volumes and fraction dry weight/volume concentrations were applied to test conditions. At 250 μg/ml, fraction 100% P1 reduced S. aureus induced cytotoxicity from 43.4±12.82% to 31.8±2.24%, however, this protective effect was lost at concentrations below 200 μg/ml. Furthermore, decreasing DMSO concentrations appeared to protect Vero cells from S. aureus, significantly reducing cytotoxicity by 24% at 50 μg/ml (0.5% v/v) (FIG. 7B). In the absence of a S. aureus challenge, fraction 100% P1 was less cytotoxic than commercial surfactin and exhibited a decreasing trend in line with the DMSO vehicle (FIG. 7C). In fact, fraction 100% P1 was significantly less cytotoxic than commercial surfactin at all concentrations except for 200 μg/ml (P=0.09). Conversely, B. subtilis 6D1 CFE applied to Vero cells at 20% v/v, 10% v/v, and 5% v/v, inhibited S. aureus ATCC 29213 induced cytotoxicity by 80.7%, 73.5%, and 61%, respectively (FIG. 7A). Based on these data we sought to investigate whether the ameliorative effects observed with B. subtilis 6D1 CFE were maintained in the human intestinal cell line, HT29 and quantify immunological markers that may be driving this effect. Application of CFE exhibited a similar dose dependent protective effect in HT29 cells challenged with S. aureus ATCC 29213, reducing cytotoxicity by 70.5%, 46.3%, and 24.5% when applied at 20% v/v, 10% v/v, and 5% v/v, respectively (FIGS. 7D and 23). Further investigation revealed this protective effect may be driven by the production of anti-inflammatory cytokine IL-10 and reduced production of the pro-inflammatory cytokine, TNFα. (FIG. 7E-F).

Example 8 Bacillus subtilis Maintains Antibiofilm Activity Against Staphylococcus aureus Following Adaptive Laboratory Evolution

### Methods

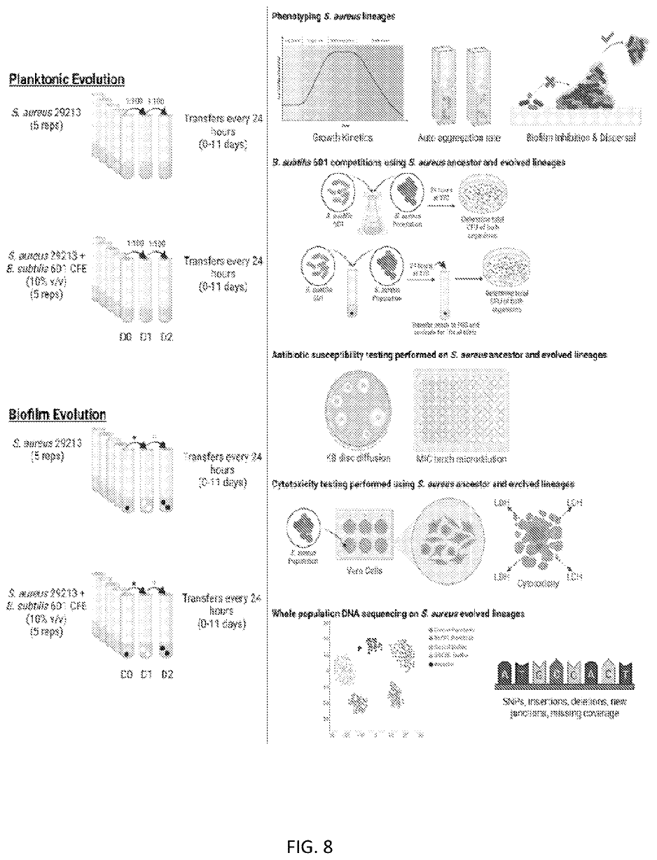

Culture conditions and preparation of B. subtilis 6D1 cell free extracts: B. subtilis 6D1 cell free extracts were prepared similar to the described Methods above. Briefly, exponential phase Bacillus cultures grown in tryptic soy broth (TSB) were individually cultured in 125 mL baffled shake flasks containing 25 mL TSB. Flasks were incubated at 37 °C shaking at 230 RPM for 48 hours. After incubation, cultures were centrifuged at 4 °C at 10,500 RPM for 20 minutes. Cell free extract (CFE) was harvested by filter-sterilization using a 0.22 μm surfactant free cellulose acetate (SCFA) filter (Corning, USA). CFEs were flash frozen in liquid nitrogen and stored at −80 °C for future use. Long term adaptive evolution of S. aureus ATCC 29213: Long term evolution experiments adapted from Santos-Lopez et al (47) and Travisano (49) were conducted using 10% v/v B. subtilis 6D1 CFE. Daily passages of S. aureus ATCC 29213 planktonic cells and biofilm cultures were performed for about 73 generations (11 days) resulting in an approximately 99% probability of obtaining a mutation at any given site within the 2.8 Mb S. aureus ATCC 29213 genome (98,99). This genome is comprised of a 2.76 Mb chromosome (GenBank sequence: CP078521.1) and 4.69 Kb plasmid (GenBank sequence: CP078522.1). FIG. 8 outlines the experimental design. Briefly, five independently evolved S. aureus populations exposed to either 10% v/v PBS or B. subtilis 6D1 CFE were passaged daily into 5 ml of fresh 1.5% TSB+0.3% glucose media. (115, 116) Planktonic cultures were diluted 1:100 during each passage. In biofilm cultures, a single 7 mm polystyrene bead was transferred daily to recapitulate the entire biofilm life cycle. All cultures were incubated in a roller drum for 24 hours at 37 °C between passages. Populations were preserved every other day and kept at −80 °C for future use. Population size was determined via CFU plating on TSA at days 1, 6, and 11.

Growth Assays: All lineages were revived 1:100 in evolution media (1.5% TSB+0.3% glucose) and seeded at 5×10⁵ CFU in 200 μl evolution media. Growth kinetics assays were performed in 96 well plates (Corning, USA) using a Multiskan GO spectrophotometer (Thermo Scientific, USA). Plates were incubated at 37 °C in the instrument and shook orbitally for 10 seconds prior to each read. Growth curves were subtracted from baseline based on each population's initial turbidity. Endpoint cell density assays differed in that they were continuously shaken at 150 RPM in a 37 °C incubator for 24 hours prior to reading turbidity. Each lineage was assayed in quadruplicate and averaged by treatment/lifestyle.

Sedimentation Assays: Sedimentation assays were conducted according to previous methods (117). To avoid the auto aggregative effects driven by media composition or pH (118), all populations were standardized in Gibco™ PBS, pH 7.4 (Thermo Scientific, USA). Briefly, each evolved S. aureus population was revived in evolution media (1:100) and incubated at 37 °C overnight at 200 RPM. Each culture was then centrifuged for 20 minutes at 4000 RPM, decanted, and standardized in PBS to ~1×10⁹ CFU/mL (1.5 OD₆₀₀). Each standardized PBS culture was centrifuged for an additional 20 minutes at 4000 RPM, vortexed for 30 seconds and 1 mL was pipetted into a cuvette. Turbidity (OD₆₀₀) was measured immediately using a Multiskan GO spectrophotometer (Thermo Scientific, USA). Cuvettes were kept at room temperature between reads. Percent auto aggregation was expressed as a function of the culture turbidity at Time 0. Each lineage was assayed in triplicate in the presence and absence of 10% v/v B. subtilis 6D1 CFE and averaged per treatment/lifestyle.

Competition Assays: For planktonic competitions, 250 μL (5×10⁷ CFU) of exponential phase B. subtilis 6D1 and S. aureus populations were added to 25 mL TSB in a baffled shake flask and incubated at 37 °C shaking at 200 RPM. Biofilm competitions utilized 7 mm polystyrene beads as a surface for cell attachment, biofilm growth, and biofilm dispersal (15). For biofilm competitions, equal concentrations of B. subtilis 6D1 and S. aureus populations (~1×10⁵-1×10⁶) were added to a glass test tube containing 5 mL 1.5% TSB+0.3% glucose media (180) and a single polystyrene bead (181,182). After 24 hours incubation at 37 °C in a roller drum, the bead was placed in 1 mL PBS and sonicated with a handheld Qsonica model CL-188 instrument (QSonica, USA) for 10 seconds at 60 Hz to remove attached cells. For all competitions, CFU counts were obtained for both bacterial species at time point 0 and after 24 hours incubation. Strain fitness and selection rate were calculated as previously described by Travisano and Lenski (183).

Cytotoxicity Assays: CCL81 (Vero) and HT29 cells were cultivated in Gibco™ Dulbecco's Modified Eagle Medium (DMEM) (Thermo Scientific, USA) consisting of 10% fetal bovine serum and 0.1% antibiotic. Cells were cultured for 7 days at 37 °C in a carbogen (95% 02, 5% CO2) atmosphere, with the culture medium being changed every two to three days until cells were confluent. Approximately 2×10⁴ cells were seeded into new 96 well plates (Corning, USA) and allowed to form monolayers for 48 hours prior to cytotoxicity testing. After removing media, cells were washed twice with Gibco™ PBS, pH 7.4 (Thermo Scientific, USA) and equilibrated in EC buffer (135 mM NaCl, 15 mM HEPES, 1 mM MgCl₂, 1 mM CaCl₂)) for four hours before adding bacteria. Exponential phase S. aureus ATCC 29213 cultures grown in 1.5% TSB+3% glucose were standardized to OD₆₀₀ 1.0, centrifuged, washed with PBS, and applied to appropriate test wells at a final concentration of 1.37×10⁷ CFU/well. Infected cell lines were incubated at 37 °C in a carbogen environment for two hours prior to conducting cytotoxicity assays using a lactate dehydrogenase (LDH) kit (Abcam, UK) as previously described(104). After treatment, cells were centrifuged at 600 g for 5 minutes. Cytotoxicity was determined using a lactate dehydrogenase (LDH) assay kit and was normalized to 100% using a cell lysis positive control solution according to the manufacturer's protocol (Abcam, UK). Three independent experiments were performed per S. aureus lineage and averaged by treatment/lifestyle. Cell lines were imaged at 20× magnification using a Nikon TMS inverted phase contrast microscope (Nikon Corporation, Japan).

Antibiotic Susceptibility Testing: Both Kirby Bauer disc diffusion and broth microdilution assays were conducted on Mueller Hinton (MH) media (BD Life Sciences, USA) (100). S. aureus populations were seeded in evolution media (1:100) and incubated at 37 °C at 200 RPM. 1.5×10⁸ CFU/mL of exponential phase S. aureus population cultures (OD₆₀₀=0.6) were spread onto square MH agar plates and allowed to dry. Sensi-Discs™ infused with known concentrations of nine different antibiotics (Table 9) were applied to the agar with sterile forceps and incubated at 37 °C for 24 hours. The resulting zones of inhibition were measured (mm) and designated as susceptible, intermediate, or resistant according to the manufacturer's guidelines for Staphylococcus. Broth microdilutions were conducted using reduced antibiotic concentrations to assess differences in minimum inhibitory concentration (MIC) below those found in the infused discs. In these experiments, 100 μl MH broth harboring different concentrations of antibiotics was inoculated with 5×10⁵ CFU/mL exponential phase S. aureus population cultures (OD₆₀₀=0.6) in 96-well polystyrene plates and incubated at 37 °C at 150 RPM for 24 hours. All experiments were performed in triplicate and compared to the ancestor.

TABLE 9
Antibiotics and their mechanisms of action used to
quantify S. aureus population sensitivity

| Antibiotic | Target | Class | Type |
|---|---|---|---|
| Ceftazidime | Penicillin binding protein | Cephalosporin | Cell wall |
| | | (3^{rd} Generation) | synthesis |
| Cefotaxime | Penicillin binding protein | Cephalosporin | inhibitors |
| | | (3rd Generation) | |
| Vancomycin | Peptidoglycan cross-linking | Glycopeptide | |
| Gentamicin | Ribosome 30S subunit | Aminoglycoside | Protein synthesis |
| Erythromycin | Ribosome 50S subunit | Macrolide | inhibitors |
| Tetracycline | Ribosome 50S subunit | Tetracycline | |
| Sulfamethoxazole/ | Folate synthesis | Sulfonamide | Nucleic acid |
| Trimethoprim | | | synthesis |
| Levofloxacin | DNA gyrase/topoisomerase IV | Fluoroquinalone | inhibitors |
| Rifampin | RNA polymerase | Ansamycin | |

Whole Population Genome Sequencing: DNA was extracted from Day 11 populations (n=20, 5/treatment) using Qiagen DNeasy Blood and Tissue Kit (Qiagen, Germany) adapted from the manufacturer's instructions to include a lysostaphin lysis step (101). Whole population genome sequencing (650 Mbp) was performed on S. aureus populations using an Illumina NextSeq.

Variant analyses assessing differences in mutation presence and frequency between the S. aureus ancestor (GenBank Accession #ASM2287046v1) and evolved populations were performed on 2×151 bp paired-end reads using breseq (v0.37.1) in polymorphism mode (103). These parameters call mutations only if they are present within the population at a frequency of at least 5% and are in at least 2 reads from each strand. Filtering, allele frequencies, and plotting were done in R software (v3.5.3) with the packages ggplot2 (v2.2.1) and tidyr. All sequencing reads were deposited in the NCBI sequence read archive (SRA) under BioProject accession number PRJNA1006038. Protein sequences harboring missense mutations were mapped onto Alphafold2 (119) predicted structures and visualized using Pymol (120).

(i) B. subtilis 6D1 CFE Maintains Antibiofilm Activity Against all Evolved S. aureus Populations Regardless of Treatment or Lifestyle

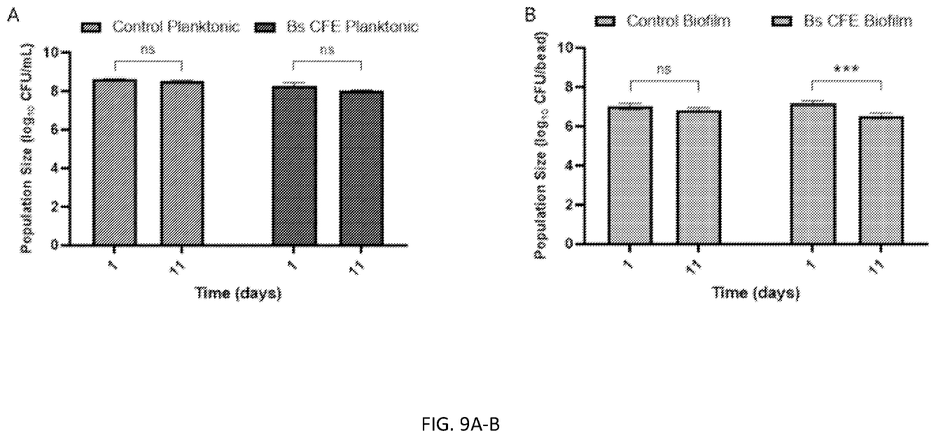

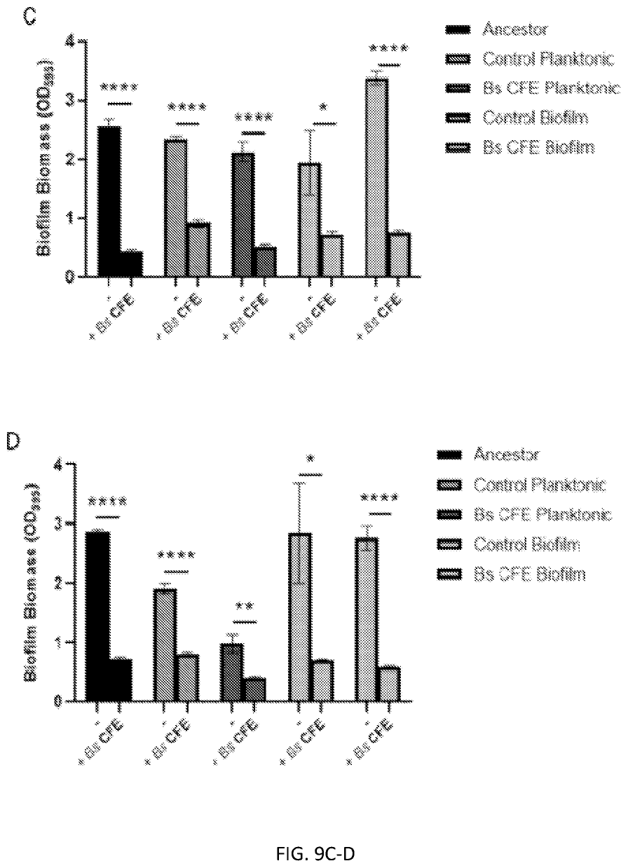

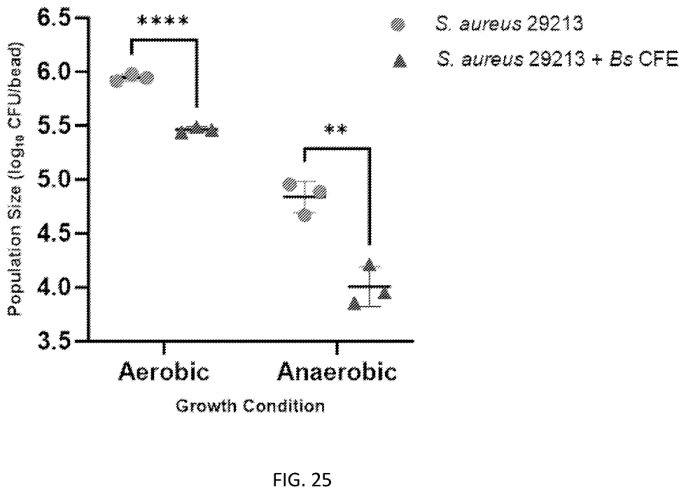

To investigate how S. aureus adapted to the evolutionary conditions, population size was determined for all treatment replicates and averaged by treatment/lifestyle after 24 hours, and at the end of the 11-day directed evolution experiment. Neither the control nor the B. subtilis 6D1 CFE (Bs CFE) exposed planktonic populations differed in population size throughout the experiment (FIG. 9A). However, Bs CFE treated biofilm lineages reduced population sizes from 7.19±0.11 to 6.52±0.13 log₁₀ CFU/bead after 11 days (FIG. 9B). To avoid bottlenecking in biofilm populations as a result of CFE exposure, beads were not rinsed prior to population transfers or enumerations. Therefore, the high cell density observed on beads after CFE exposure can likely be attributed to an increased number of unattached cells being included in our CFU counts (FIG. 25). Despite these changes in population size, growth rate assays conducted in the same evolution media revealed mean doubling time decreased for all lineages compared to the ancestor, an indication that evolved populations positively adapted to the media composition used throughout the experiment (Table 10).

TABLE 10
Evolved lineages exhibit improved growth rates
in evolution media compared to the ancestor

| Treatment/ | Slope | Mean Exponential | Mean Doubling |
|---|---|---|---|
| Lifestyle | (R²) | Growth Rate (μ) | Time (min) |
| Ancestor | 0.9912 | 0.64 | 84.28 |
| Control Planktonic | 0.9938 | 0.93 | 63.42 |
| Bs CFE Planktonic | 0.9967 | 0.95 | 62.2 |
| Control Biofilm | 0.9945 | 0.98 | 60.46 |
| BS CFE Biofilm | 0.9963 | 0.89 | 65.46 |

Biofilm inhibition and dispersal assays were performed to identify whether evolved lineages developed resistance to the antibiofilm activity of B. subtilis 6D1 CFE. When applied alongside S. aureus populations at TO, 10% v/v CFE reduced mean biofilm formation of control planktonic, Bs CFE planktonic, control biofilm, and Bs CFE biofilm populations by 61%, 75%, 62%, and 78%, respectively (FIG. 9C). Comparatively, ancestral biofilm growth decreased by 83% when grown in the presence of B. subtilis 6D1 CFE. Biofilm dispersal assays also revealed 10% v/v CFE improved dispersal compared to biofilms similarly treated with PBS. When applied to day old mature S. aureus biofilms for two hours, 10% v/v CFE increased mean biofilm dispersal of control planktonic, Bs CFE planktonic, control biofilm, and Bs CFE biofilm populations by 58%, 58%, 75%, and 79%, respectively (FIG. 9D). Meanwhile, 10% v/v CFE increased biofilm dispersal by 75% in the ancestor. Collectively, these results demonstrate 10% v/v B. subtilis 6D1 CFE maintains biofilm inhibition and dispersal activity in all treatment conditions regardless of long-term CFE exposure or lifestyle condition. However, while there was no significant difference in mean biofilm biomass observed between the ancestor and each treatment condition, significantly higher biofilm biomass was achieved in the Bs CFE biofilm treatment compared to all other treatment conditions (FIG. 9C), suggesting these lineages may have adapted a slightly stronger biofilm phenotype.

(ii) S. aureus Populations Evolved in the Presence of B. subtilis 6D1 CFE Possess Stronger Auto Aggregation Capabilities but can Still be Abrogated by the Presence of Bs CFE.

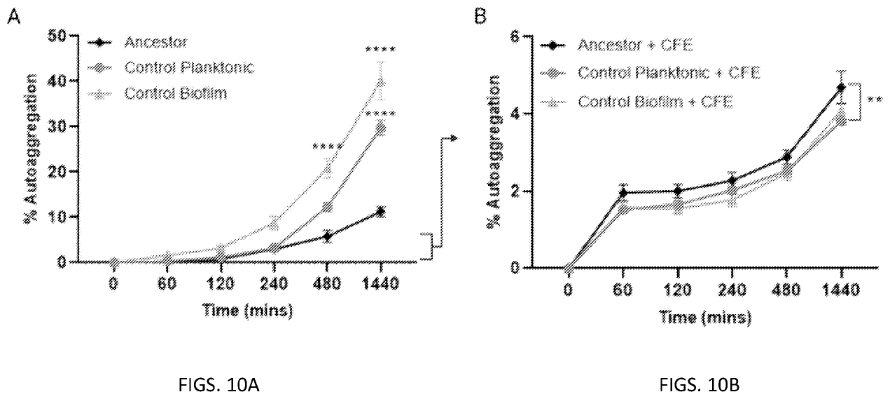

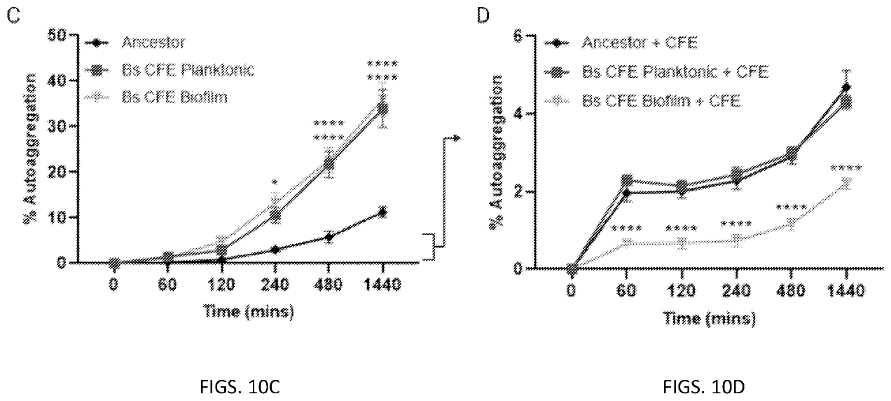

To further explore whether Bs CFE evolved lineages were more apt to form biofilm, sedimentation assays were performed to identify differences in auto-aggregation—a biofilm phenotype where cells adhere to each other rather than a surface (117). Control biofilm lineages exhibited faster and stronger aggregation compared to the ancestor and similarly evolved planktonic lineages, increasing aggregation by 20.8±2.1% and 40.1±4.2% after 8 and 24 hours, respectively (FIG. 10A). Interestingly, both CFE evolved biofilm and planktonic lineages aggregated at similar rates (FIG. 10C). In fact, Bs CFE planktonic populations achieved significantly more aggregation after only four hours compared to their similarly evolved control counterparts (P=0.031). No differences in percent auto aggregation were observed between control and Bs CFE evolved biofilm lineages at any of the timepoints tested. However, in agreement with our previous experiments, the presence of 10% v/v CFE abrogated auto aggregation in all treatment lineages (FIGS. 10B, 10D). Compared to the ancestor, however, Bs CFE biofilm lineages were significantly more susceptible to the effects of CFE at all time points (FIG. 10D), suggesting that despite evolving an enhanced aggregative phenotype, these populations were more susceptible to the antibiofilm activity of B. subtilis 6D1 CFE. These results confirm the antibiofilm activity observed in our previous experiments can also be attributed to an inhibition of cell aggregation, a phenomena that, like biofilm, affords cells protection from antibiotics (121).

(iii) S. aureus Populations Evolved in the Presence of B. subtilis 6D1 CFE are Less Competitive in a Biofilm Environment and More Competitive in a Planktonic Environment.

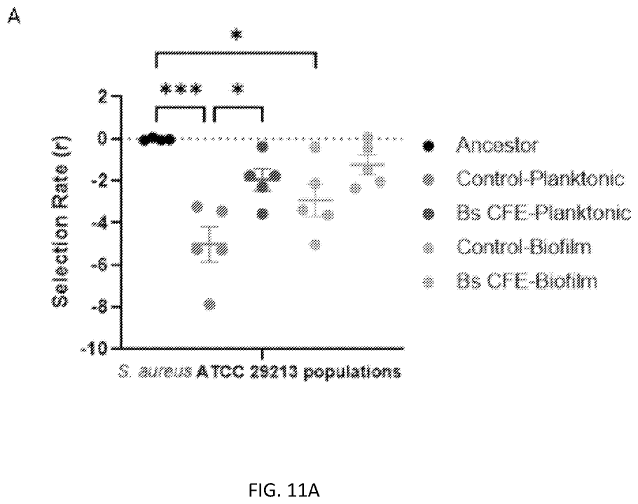

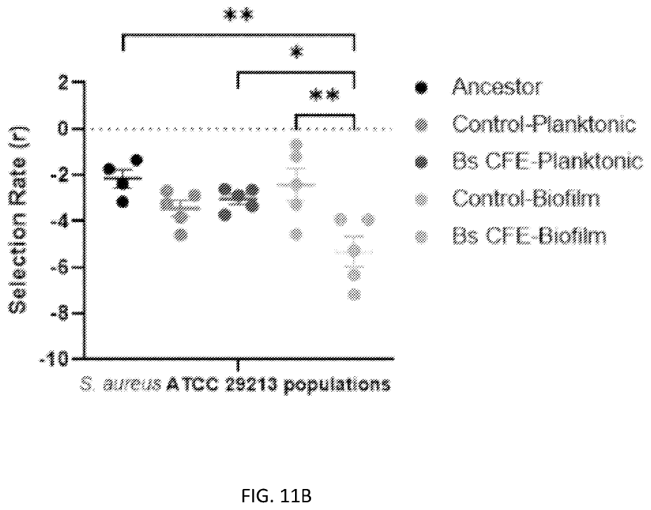

To assess how these aggregative phenotypes affected competitive fitness, planktonic and biofilm competitions were performed between evolved S. aureus populations and B. subtilis 6D1. Planktonic competitions revealed control lineages were less competitive against B. subtilis 6D1 than the ancestor (FIG. 11A). Additionally, Bs CFE planktonic lineages were more competitive than the similarly evolved untreated lineages (P=0.017). In this instance, the mean selection rate increased from −5.018±0.84 for control planktonic populations to −1.95±0.51 for Bs CFE planktonic populations. Mean selection rates also increased from −2.91±0.78 to −1.24±0.46 for control and Bs CFE treated biofilm lineages, respectively, albeit these differences were not significant (P=0.33). In biofilm competitions, our data revealed only Bs CFE biofilm lineages were less competitive when compared to the ancestor (FIG. 4B). In fact, Bs CFE biofilm lineages (−5.32±0.65) were less competitive than both similarly evolved control biofilm populations (−2.42±0.70) and Bs CFE planktonic lineages (−2.64±0.21). In addition to being more susceptible to CFE anti aggregation activity, Bs CFE biofilm lineages are also less completive against B. subtilis in a biofilm. Collectively, these data align with our initial findings showing Bs CFE treated biofilm populations reduced population size after the 11 day evolution (FIG. 11B).

(iv) S. aureus Populations Evolved in the Presence of B. subtilis 6D1 CFE are Less Virulent than the Ancestor.

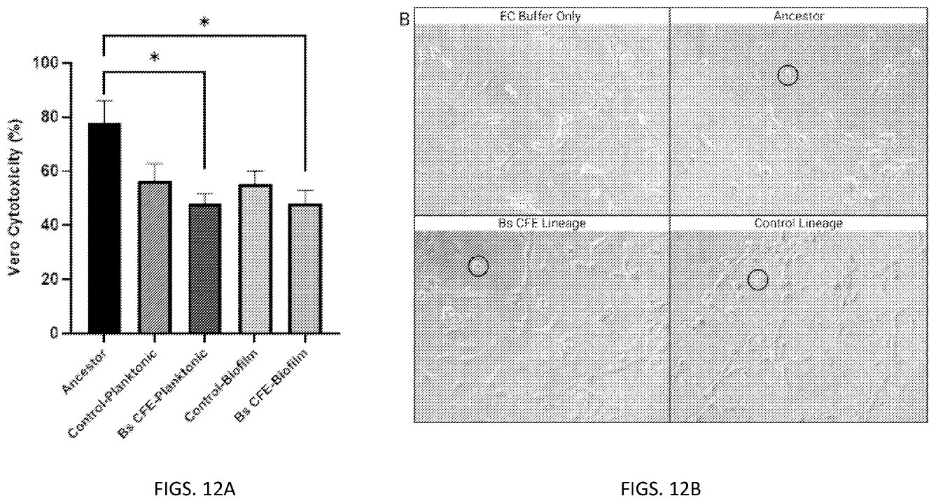

To assess whether fitness defects observed in our competition experiments correlated with an altered virulence potential, the S. aureus ancestor and all evolved lineages were applied to Vero (CCL81) cells and evaluated for cytotoxic effects via lactate dehydrogenase (LDH) release. Vero cell toxicity for both biofilm and planktonic lineages evolved in the presence of Bs CFE was 47.87±5.07% and 47.96±3.66%, respectively, roughly 30% less cytotoxic than the ancestor (77.66±8.43%) (FIG. 12A). LDH also trended lower in similarly evolved control populations, yielding 56.31±6.44% and 55.22±4.88% cytotoxicity for planktonic and biofilm lineages, respectively. Though no difference in LDH release was observed between evolutionary treatments, unhealthy Vero cell phenotypes appeared less frequently and were less pronounced in cell lines infected with CFE evolved lineages than control lineages (FIG. 12B). Together, these data confirm that fitness defects observed in our competition experiments do, indeed, correlate with a diminished virulence potential of S. aureus.

(v) S. aureus Populations Evolved in the Presence of B. subtilis 6D1 CFE do not Harbor Increased Phenotypic Sensitivity.

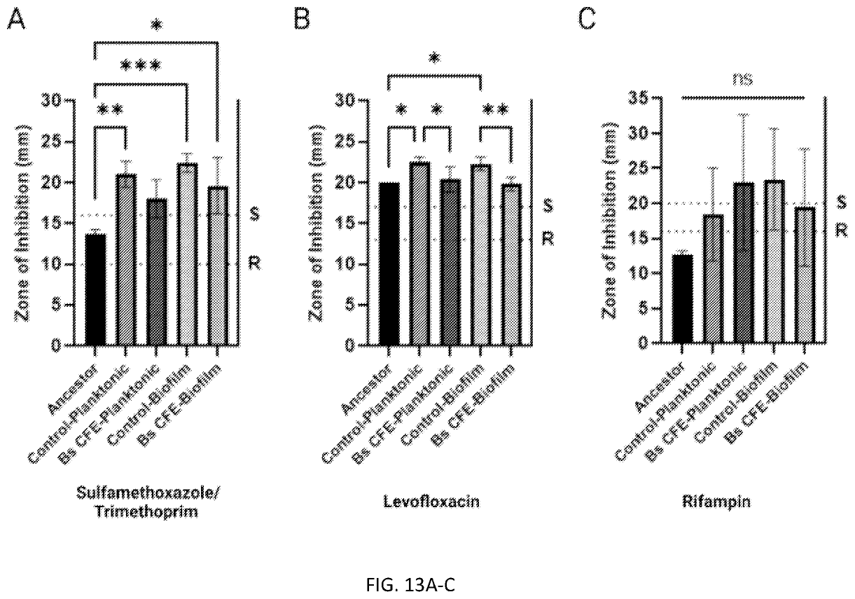

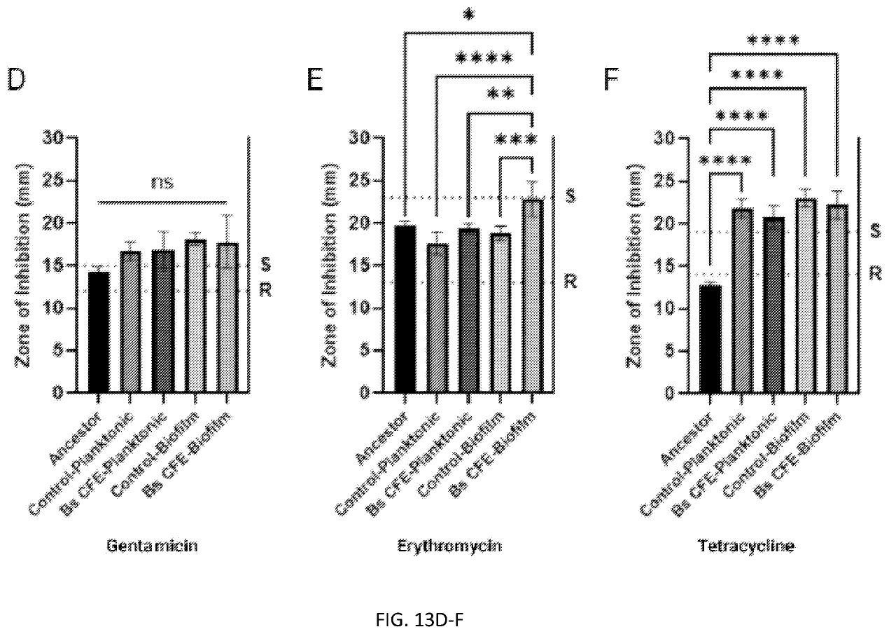

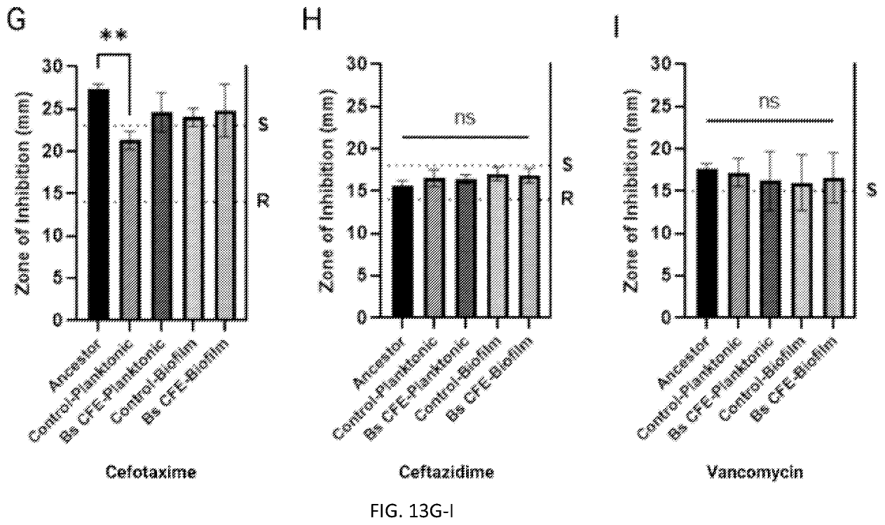

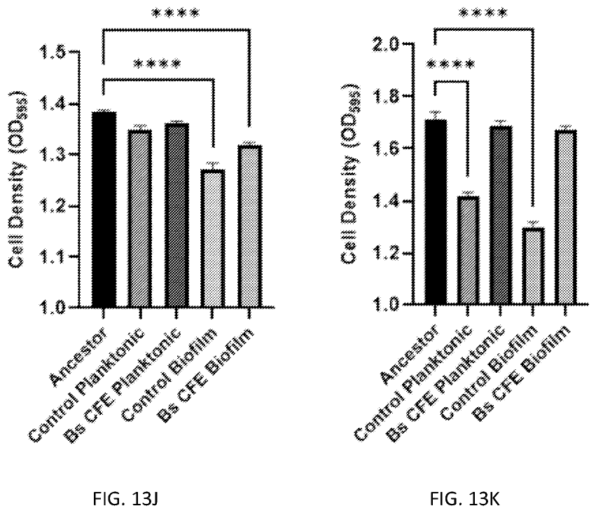

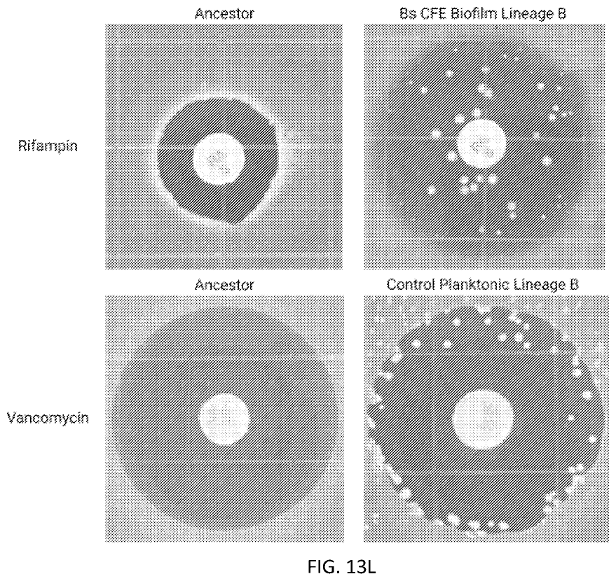

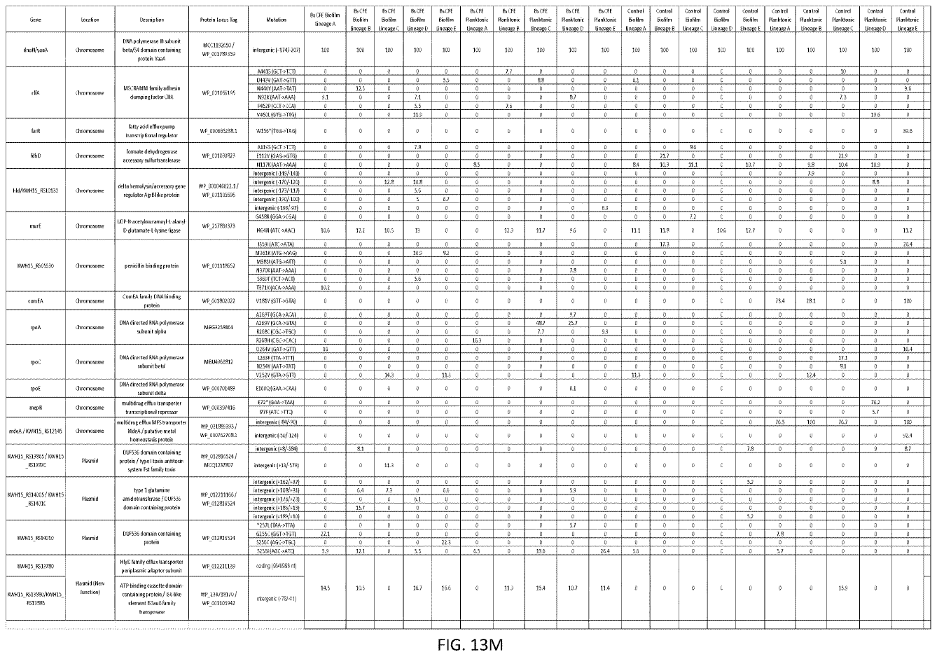

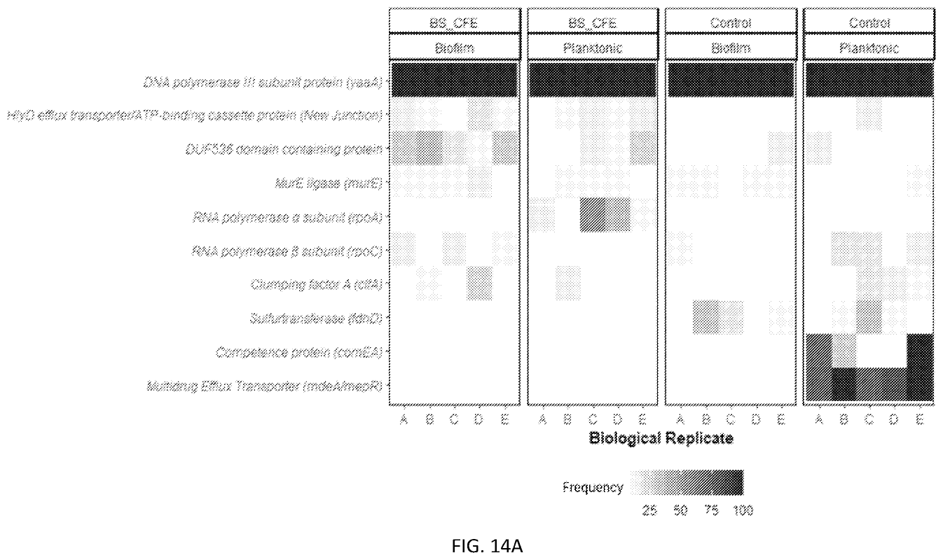

To investigate whether long-term exposure to B. subtilis 6D1 CFE generated antibiotic cross resistance, phenotypic sensitivity testing was carried out using Kirby Bauer disc diffusion and broth microdilution assays. Compared to the ancestor, all lineages became more sensitive to Sulfamethoxazole/Trimethoprim, but only control populations increased mean zone of inhibition significantly (FIG. 13A). Despite control populations exhibiting increased sensitivity to Levofloxacin, all lineages remained susceptible to this antibiotic after evolution (FIG. 13B). Surprisingly, multiple evolved lineages developed greater phenotypic sensitivity to Rifampicin (FIG. 13C), however satellite colonies emerged after three days in many of these lineages (FIG. 13L). Though no significant differences for Gentamicin were observed, the mean zone of inhibition transitioned from intermediate to susceptible for all evolved lineages (FIG. 13D). Furthermore, many of these lineages also exhibited lower MIC values (Table 11) compared to the ancestor.

TABLE 11
Minimum inhibitory concentrations of evolved S. aureus lineages exposed to various antibiotics

| | Antibiotic MIC (ug/mL) | | | | | | | |
|---|---|---|---|---|---|---|---|---|
| Population | Gentamicin | Tetracycline | Rifampin | Erythromycin | Sulfa-Trim | Cefotaxime | Ceftazidime | Vancomycin |
| Ancestor | 2.5 | 15 | 5 | 30 | 23.75 × 1.25 | 1.87 | 30 | <1.87 |
| Control Planktonic A | 2.5 | 7.5 | 5 | 30 | 23.75 × 1.25 | 3.75 | 15 | <1.87 |
| Control Planktonic B | 1.25 | 7.5 | 5 | 15 | 23.75 × 1.25 | 3.75 | 15 | <1.87 |
| Control Planktonic C | 2.5 | 7.5 | 2.5 | 30 | 23.75 × 1.25 | 3.75 | 30 | <1.87 |
| Control Planktonic D | 1.25 | 7.5 | 5 | 30 | 23.75 × 1.25 | 1.87 | 30 | <1.87 |
| Control Planktonic E | 1.25 | 15 | 5 | 30 | 23.75 × 1.25 | 1.87 | 30 | <1.87 |
| Bs CFE Planktonic A | 1.25 | 7.5 | 5 | 15 | 23.75 × 1.25 | 1.87 | 30 | <1.87 |
| Bs CFE Planktonic B | 1.25 | 7.5 | 2.5 | 15 | 23.75 × 1.25 | 0.93 | 30 | <1.87 |
| Bs CFE Planktonic C | 1.25 | 15 | 2.5 | 15 | 23.75 × 1.25 | 0.46 | 30 | <1.87 |
| Bs CFE Planktonic D | 1.25 | 15 | 2.5 | 15 | 23.75 × 1.25 | 1.87 | 30 | <1.87 |
| Bs CFE Planktonic E | 2.5 | 15 | 5 | 15 | 23.75 × 1.25 | 0.93 | 30 | <1.87 |
| Control Biofilm A | 2.5 | 7.5 | 5 | 30 | 23.75 × 1.25 | 1.87 | 15 | <1.87 |
| Control Biofilm B | 1.25 | 7.5 | 2.5 | 30 | 23.75 × 1.25 | 1.87 | 15 | <1.87 |
| Control Biofilm C | 1.25 | 7.5 | 2.5 | 30 | 23.75 × 1.25 | 3.75 | 15 | <1.87 |
| Control Biofilm D | 2.5 | 7.5 | 2.5 | 30 | 23.75 × 1.25 | 1.87 | 15 | <1.87 |
| Control Biofilm E | 1.25 | 7.5 | 5 | 30 | 23.75 × 1.25 | 3.75 | 15 | <1.87 |
| Bs CFE Biofilm A | 2.5 | 7.5 | 2.5 | 15 | 23.75 × 1.25 | 1.87 | 7.5 | <1.87 |
| Bs CFE Biofilm B | 1.25 | 7.5 | 2.5 | 15 | 23.75 × 1.25 | 0.93 | 7.5 | <1.87 |
| Bs CFE Biofilm C | 2.5 | 15 | 5 | 15 | 23.75 × 1.25 | 1.87 | 15 | <1.87 |
| Bs CFE Biofilm D | 2.5 | 7.5 | 5 | 15 | 23.75 × 1.25 | 1.87 | 15 | <1.87 |
| Bs CFE Biofilm E | 1.25 | 7.5 | 5 | 15 | 23.75 × 1.25 | 0.93 | 15 | <1.87 |

All evolved populations remained intermediately resistant to Erythromycin, however biofilm populations evolved in the presence of Bs CFE were more sensitive than all other populations in both KB disc assays (FIG. 13E) and MIC assays (Table 11). Interestingly, all populations were more susceptible, both numerically and categorically, to tetracycline (FIG. 13F), and 75% of populations had lower MIC values compared to the ancestor (Table 11). Control planktonic lineages were the only populations to exhibit increased resistance to cefotaxime (FIG. 13G), and although no differences were observed among populations using the KB disc assay, lower ceftazidime MIC values were observed for all biofilm lineages (FIGS. 13H, 13M). Though no sensitivity differences were observed after Vancomycin exposure (FIG. 13I), four lineages from three different treatment conditions yielded satellite colonies after extended incubation (FIG. 13L). Lastly, it is possible that the composition of MHB contributed to increased sensitivity patterns, since control lineages, but not CFE lineages, succumbed to fitness defects when grown in MHB (FIGS. 13J, 13K). Therefore, sensitivity patterns observed in Bs CFE evolved lineages were not simply an artifact of reduced fitness in MHB media.

(vi) Population Sequencing Reveals Treatment and Lifestyle-Associated Molecular Targets of S. aureus Evolution.

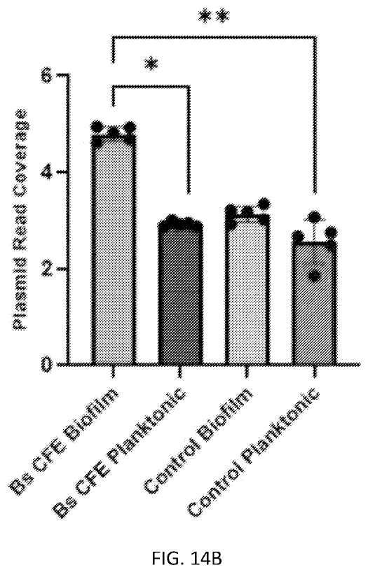

Whole population genome sequencing was performed to identify genetic mutations that might explain the phenotypic characterisitics observed across S. aureus evolved lineages. Collectively, in addition to discernible parallelism observed across treatment replicates, our results revealed mutations incurred by S. aureus ATCC 29213 after about 73 generations followed treatment and lifestyle associated patterns (FIG. 14). Mutations identified in the 2.76 Mb chromosome (GenBank sequence: CP078521.1) affected genes associated with cell surface proteins-namely efflux pumps, competence proteins, clumping factors, and peptidoglycan synthesis enzymes. Mutations were also detected in genes driving RNA polymerase (RNAP) assembly and function.

Interestingly, control planktonic-evolved lineages had the clearest life-style mutational parallelism. A nonsense mutation yielding a premature stop codon was found in mepR (WP_000397416), at residue E72, which truncates MepR 67 amino acids shorter than the full-length protein. This truncation likely abolishes protein activity since hydrophobic residues L76, 177, L93, 198, L100 and V101 that make up the alpha side chain are critical (122). Though no mutations occurred within the mdeA (WP_031889393) coding region, single point mutations (C→A) were identified in four of five lineages 84 bp upstream of the mdeA translation start site (FIG. 13M). Either mdeA or mepR mutations were found at high frequency (>76.2%) in all control planktonic lineages. Mutations in competence pathways were also observed in this treatment group. A synonymous comE (WP_001802022) point mutation (T→A) at residue V181 was detected in control planktonic lineages A, B, and E, at 73.4%, 28.1%, and 100% frequency, respectively (FIG. 14). Natural competence promotes genetic variability and provides S. aureus with additional nutritional and metabolic possibilities, allowing it to proliferate during infection (123).

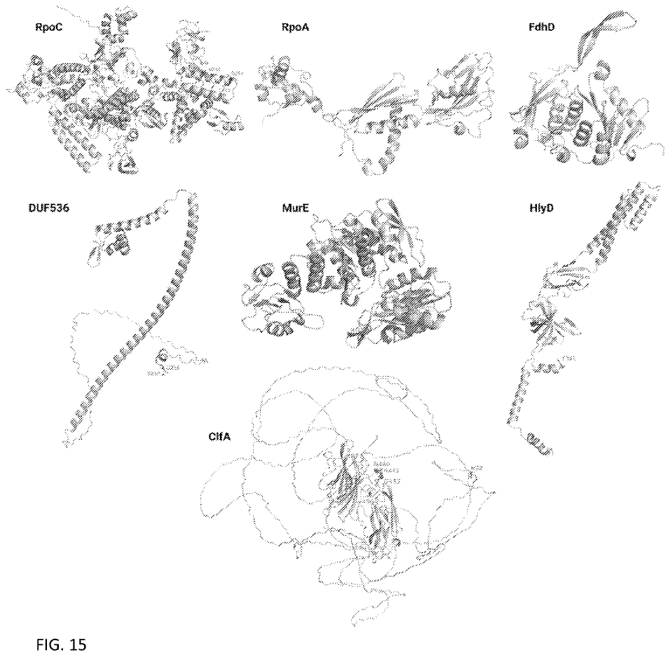

Clumping factor A (WP_001056195), a cell-wall-anchored protein and a virulence factor in various infections incurred mutations at A441S, D443V, N440Y, N92K, P452P, and V450L. These were found in both CFE treated conditions and control planktonic lineages albeit at varying frequencies and with no apparent association between the residue alteration and evolutionary condition. Mutations in MurE (WP_267830373), which encodes an enzyme critical for maintaining peptidoglycan integrity, were also identified predominantly in biofilm and Bs CFE-evolved lineages. A missense murE mutation (C→A) at residue 464 converted isoleucine to asparagine in all afflicted lineages except in control biofilm C where a point mutation (C→A) converted residue 458 from a glycine to an arginine (FIG. 13M). Both positions are located in the main chain of the MurE protein structure (FIG. 15) and involved in L-lysine substrate interactions (124). Mur ligases help to remodel and recycle peptidoglycan in S. aureus, and this recycling is known to drive bacterial aggregation and biofilm formation (156). Therefore, it is possible mutations identified in murE are contributing to the increased aggregation phenotype observed in biofilm evolved and Bs CFE-evolved lineages.

FdhD (WP_001030823) mutations encoding the membrane bound formate dehydrogenase enzyme occurred predominantly at N117K and E112V, and almost exclusively in control lineages (FIG. 13M). Of note, a farE missense mutation was identified at 40% frequency in control planktonic E (FIG. 13M). FarR is required to promote farE expression whose product encodes a resistance-nodulation-division (RND) family efflux pump that is activated in response to the antimicrobial linoleic acid (125).

In addition to mutations affecting cell surface proteins, pathways affecting RNA polymerase (RNAP) structure and function were also identified in a treatment/lifestyle pattern. Specifically, four Bs CFE planktonic lineages harbored non-synonymous rpoA (MBG3258464) mutations at residues A269 and R268 (FIG. 13M). The a subunit encoded by rpoA plays an important role in RNAP assembly since its dimerization is the first step in the sequential assembly of subunits to form the holoenzyme (126). Lineage D also harbored a low frequency mutation (E160Q) in rpoE (WP_000701483) encoding the RNAP δ subunit which is essential for cell survival when facing a competing strain in a changing environment (127). RpoC (MBU4966812) mutations V252V and D264V were found more frequently across affected lineages followed by L263F and N254Y, however only missense mutations were found in Bs CFE biofilm and control planktonic lineages. RpoC encodes the β′ subunit which together with the a dimer and B subunit, form the catalytic center of RNAP. The predicted structure of RpoC (A0A0E1VM47_STAA3) suggest the mutations identified in our experiment occur at residues with high model confidence (pLDDT>90) (FIG. 15). Intergenic mutations were also identified in Bs CFE biofilm lineages between agrB and hld albeit at low frequency (FIG. 13M). However, recent work suggests frameshift mutations that occur in the Agr operon are evolutionarily short lived (128). Media specific parallelism was observed across all treatments, including controls, related to DNA polymerase function. All lineages incurred intergenic point mutations (A→T) at 100% frequency 174 bp downstram of dnaN, which encodes a DNA beta sliding clamp, and 207 bp upstream of yaaA at position 3491, which encodes a S4 domain containing protein.

Breseq also identified mutations in the circular 4.69 Kb plasmid (GenBank sequence: CP078522.1). Specifically, mutations in the DUF536 domain containing protein (WP_012816524), identified more frequently in CFE exposed lineages, were almost exclusively incurred at sites G255 and S256, yielding cysteine and isoleucine alterations at the last two amino acid residues of this protein domain, respectively. Moreover, multiple SNPs were identified 8-37 nucleotides downstream of DUF536 (FIG. 13M). Domains of unknown function (DUFs) are a large set of uncharacterized protein families(129), however a quick blast search of this protein suggests this protein may be involved in replication events. Furthermore, its predicted Alphafold structure (D2J7G1_STAUU), reveal the two residues affected by missense mutations may play an important role in how this protein interacts in the environment (FIG. 15); however, both residues harbor low model confidence scores (pLDDT<63). Perhaps most surprising were the new junctions yielding head to head mutations in CFE evolved lineages. Beginning at position 9348 or 9350 in the coding region transcribing the HlyD family efflux transporter periplasmic adaptor subunit (WP_012211139), these junctions typically ended 76 or 78 base pairs upstream of the gene encoding ATP binding cassette domain containing protein (WP_234718170) (FIG. 13M). The predicted structure of HlyD (A1KWU5_STAAU) suggests this protein may be a periplasmic component of an efflux system, and similar to DUF536, truncation of the outward facing 331^{st} residue by this new junction may play an important role in how this protein interacts in the environment (FIG. 15).

### Discussion

The research goal set out to understand how Bacillus strains with the propensity to inhibit S. aureus biofilm formation achieve this effect through quorum sensing interference. Furthermore, the research sought to identify whether compounds produced by these Bacillus strains were capable of inhibiting biofilm formation in each of the four currently characterized S. aureus Agr QS backgrounds. The results demonstrate a single strain, herein referred to as B. subtilis 6D1, possesses unique abilities to inhibit biofilm growth, disassemble mature biofilm, and improve antibiotic sensitivity of S. aureus biofilms (FIGS. 2A-2H) through an Agr quorum sensing interference mechanism (FIGS. 3A-3B). In addition to harboring unique biosynthetic gene clusters (FIG. 4I), LC-MS/MS and molecular network analysis revealed that the B. subtilis 6D1 100% P1 fraction, harboring surfactin A-D and an unidentified network was driving this antibiofilm activity (FIG. 5B-C). Further comparative analysis confirmed at 500 μg/ml concentrations, this fraction exhibited stronger antibiofilm activity against S. aureus than commercial surfactin obtained from B. subtilis alone (FIG. 6B-E), and also decreased biofilm formation in a number of clinical S. epidermidis strains, while commercial surfactin did not have this effect (FIG. 6F). Lastly, B. subtilis 6D1 can stimulate the human immune response in a manner that reduces S. aureus-induced intestinal epithelial cell toxicity.

Application of B. subtilis 6D1 CFE inhibited S. aureus biofilm growth and removed mature biofilm in a dose dependent manner (FIG. 2C-D) without inhibiting planktonic growth (FIG. 1A), providing the first evidence that compounds produced by this strain were capable of quorum sensing interference(77). Centrifugal filtration of crude CFEs confirmed antibiofilm activity was maintained in fractions <3 kDa (FIG. 2H), suggesting that small peptides were likely driving this effect. Similar antibiofilm activity was observed in B. subtilis 6D1 CFEs obtained from MRS and defined media (data not shown), suggesting compound production is not restricted to growth only in nutrient rich TSB. Moreover, B. subtilis 6D1's ability to outcompete S. aureus ATCC 29213 in a biofilm but not in a planktonic environment further suggest that competitive interactions between these strains likely did not deter production of these antibiofilm compounds (FIG. 1C). Therefore, it's tempting to speculate that competition between B. subtilis 6D1 and S. aureus ATCC 29213 likely involve either competitive exclusion or niche partitioning mechanisms rather than a tit-for-tat bacterial warfare mechanism whereby one strain produces antimicrobial compounds to destroy the perceived threat of nother (78). While these competitive benefits observed in B. subtilis 6D1 may be diminished or abrogated in a more complex microbial environment, previous work conducted by Piewngam and Qiu suggest these effects may indeed persist in complex environments like the human gut and sites of S. aureus infection(79). In fact, B. subtilis 6D1's inability to inhibit S. aureus planktonic growth was also observed in B. subtilis H28, the strain used by Qiu and colleagues to successfully alleviate S. aureus-induced mastitis infection in a murine model(42). Furthermore, the synergistic effects of B. subtilis 6D1 CFE when applied alongside Gentamicin and Ampicillin (FIG. 2E-F), aligns with previous studies investigating the improved inhibitory effects seen when B. subtilis CFE was paired with Gentamicin and Penicillin to treat an osteomyelitis infection in mice(80). Further efforts will investigate whether application of B. subtilis 6D1 reduces S. aureus biofilm formation and virulence in a polymicrobial environment.

Gene expression results further helped to explain why both B. subtilis 6D1 CFE and cell to cell interactions yielded similar antibiofilm activity against S. aureus ATCC 29213. Specifically, these experiments showed both B. subtilis 6D1 culture and its associated CFE increased expression of the same Agr and stress specific genes in S. aureus ATCC 29213 (FIG. 3A). In addition to agrA being upregulated, expression of both RNAIII and hld indicate the phosphorylation of AgrA is prompting the transcription of both the P2 and P3 promoters of the Agr regulon. Recent work has identified that overexpression of RNAIII contributes to cell lethality in Agr positive S. aureus strains (81), suggesting B. subtilis 6D1 might induce cell death indirectly if exposure is increased beyond 24 hours and RNAIII expression increases. In addition to upregulating agrA, RNAIII, and hld, application of B. subtilis 6D1 and its associated CFE also increased the expression of sigB and saeR. Interestingly, both SigB and the SaeR/S system are essential to persist intracellularly and evade the host innate immune system (82,83). However, to accomplish this, S. aureus also needs to silence Agr, and, in doing so, adopt a biofilm lifestyle(84). The Sae-regulon also includes genes associated with biofilm formation (nucleases) and dispersal (proteases) factors; therefore, it is possible that different compounds produced by B. subtilis 6D1 might also trigger the expression of the Sae system independently of the Agr system and negatively impact S. aureus biofilm formation through the production of proteases(69). However, previous work has shown that when S. aureus is grown in TSB, the Sae system was found to be dispensable for biofilm formation (85), so it is unlikely that Sae was the primary driver of biofilm formation given our experimental conditions. Furthermore, previous work demonstrated a sae mutation did not affect the expression of agr, it is proposed that the saeR/S system modulates the production of virulence factors downstream of agr(174-175). Though it was surprising to observe similar S. aureus gene expression responses after expose to B. subtilis 6D1 CFE and live cells, it is possible these responses were driven by different compounds than those driving the antibiofilm activity. Specifically, Lauderdale and colleagues demonstrated that agr-induced RNAIII levels were elevated in sigB mutants(86), however this implies sigB expression should be reduced if agrA expression increases. This assumption contradicts the findings; however, and this difference can be attributed to multiple different compounds independently altering S. aureus gene expression associated with biofilm formation and the global stress response. Nevertheless, it is speculative that QSI strategies employed by probiotic bacteria that facilitate an Agr ‘on’ state might inadvertently reduce the risk of chronic S. aureus infections (26,27).

This is the first disclosure showcasing B. subtilis derived peptides exhibiting broad antibiofilm activity against all four S. aureus Agr backgrounds. Furthermore, antibiofilm activity against a strain unable to produce AgrD, RN7206, suggest that B. subtilis derived surfactin compounds are binding AgrC and initiating the phosphorylation of AgrA (FIG. 3B). Antibiofilm activity of 100% P1 against multiple S. epidermidis strains was also somewhat surprising (FIG. 6E). S. epidermidis also harbors an Agr system that succumbs to QSI and cross-talk between similarly structured AgrD configurations(87), however, the AgrD peptide variants found in S. epidermidis are structurally different than those found in S. aureus(88). Despite this, it has been reported that peptides capable of interfering with the S. aureus Agr system are also capable of interfering with the S. epidermidis Agr systems(63), suggesting peptides produced by B. subtilis 6D1 may possess broader QSI activity than tested here. Further studies using purified compounds rather than crude or refined fractions will help us understand the binding affinity and specificity of these compounds to a variety of AgrC receptors found in multiple Staphylococcal Agr signaling systems.

Despite not working with purified compounds, it was interesting that fraction 100% P1 obtained from B. subtilis 6D1 harbored greater and broader antibiofilm activity than that of commercial surfactin also obtained from B. subtilis (FIGS. 6B-6F). It is possible these differences are due to the presence and increased concentration of multiple surfactin isoforms in fraction 100% P1 (FIGS. 6A-6D) compared to the commercial surfactin that only harbors surfactin C. Alternatively, this activity may be due to the presence of the unique compound found in 100% P1, since refined fractions enriched with this compound also inhibited S. aureus biofilm growth and disassembled mature biofilm (FIGS. 27A-B).

To purify the unique compound and elucidate its structure, batch-culturing was used to increase the abundance of this compound, however the resulting concentrations were too low to deduce an accurate molecular formula or structure. We further attempted to enrich the fraction with the unique compound by knocking out the surfactin gene cluster in B. subtilis 6D1 and obtaining new extracts without any surfactins. However, attempts to knockout the surfactin gene cluster using natural transformation, SPP1 phage transduction, and transposon insertion mutagenesis (89) were all unsuccessful. Further genomic analysis revealed B. subtilis 6D1 has an endogenous pBS32-like plasmid known to carry genes that functionally inhibit the competence of DNA uptake machinery (70,90). Thus, this strain may have an impaired machinery to uptake foreign DNA.

While fraction 100% P1 was less cytotoxic than commercial surfactin (FIG. 7B), only 100% P1 applied at 250 μg/ml ameliorated S. aureus induced cytotoxicity in Vero cells (FIG. 7C). However, the protective and immunomodulatory effects afforded by B. subtilis 6D1 CFE in both Vero and HT29 cells indicates the consortia of molecules produced by this strain can reduce S. aureus ATCC 29213 virulence through stimulation of intestinal adaptive immunity. Moreover, HT29 monolayers treated with 20% v/v CFE harbored less LDH accumulation than untreated monolayers not challenged with S. aureus (data not shown), suggesting this consortium may improve gut epithelial integrity in unchallenged cell types. These results align with previous research demonstrating how probiotic derived small molecules, termed postbiotics, can improve gut physiological processes and adaptive immunity (91,92). HT29 cells have been frequently used to study the intestinal immune response to bacterial infection, adhesion, and survival (93), and although they represent a valuable model due to their similarities with enterocytes of the small intestine, their limitations and the relevance to the in vivo situation are still under debate. Specifically, differentiated HT29 cells express brush-border-associated hydrolases typical of the small intestine although the enzymatic activity is lower than that found in vivo.

A thorough understanding of probiotic strain mechanisms is critical if they are to serve as therapeutic options against biofilm forming pathogens like S. aureus. This work has demonstrated that B. subtilis 6D1 and the production of surfactin lipopeptides and the unique compound may serve multiple roles in preventing chronic S. aureus infection by inhibiting biofilm formation and attachment, increasing sensitivity to antibiotics, and stimulating an immune response capable of reducing S. aureus virulence and improving intestinal health. Further in vivo studies to assess the utility of this strain in complex and highly variable communities seen in the gastrointestinal tract and sites of S. aureus infection are warranted.

How long-term exposure to probiotic derived compounds with antibiofilm and QSI activity drive pathogen evolution, fitness, virulence, and antibiotic sensitivity was further investigated. After 80 generations, B. subtilis 6D1 CFE maintained antibiofilm activity and inhibited auto aggregation against all lineages regardless of treatment or lifestyle (FIGS. 9A-9D, 10B, 10D). B. subtilis 6D1 also maintained higher competitive fitness against all S. aureus lineages in biofilm (FIG. 11B). Furthermore, S. aureus lineages evolved in the presence of B. subtilis 6D1 CFE were less cytotoxic than the ancestor in a Vero cell line model of infection (FIGS. 12A-12B) and did not develop phenotypic resistance to a variety of antibiotics targeting cell wall integrity, protein translation, and nucleic acid synthesis when compared against the broadly susceptible S. aureus ATCC 29213 ancestor(130-131) (FIG. 13). However, lineages evolved in the presence of CFE harbored stronger auto aggregative phenotypes than similarly evolved control lineages and the ancestor (FIG. 10A, 10C). Bacterial auto aggregation or clumping is a process whereby bacteria physically interact with each other and settle to the bottom in a static liquid suspension. Similar to biofilms, disrupting aggregation increases antimicrobial sensitivity(118), however, aggregate formation also improves S. aureus survival in the host and increases antibiotic tolerance(121,132). Thus, aggregation is a promising target for therapeutic development. In fact, a number of small molecule inhibitors designed to inhibit aggregation also reduced S. aureus virulence(133-135), however, previous work has demonstrated that S. aureus cells can exhibit rapid, reversible aggregation if cellular hydrophobicity, surface potential or extracellular cues are recognized. Haaber et al. also showed that, in contrast to biofilms, S. aureus aggregates had 7-fold higher metabolic activity and a slightly increased mutation frequency than planktonic cells(121). Thus, auto aggregation is believed to provide bacteria with the benefits of biofilm while maintaining mobility-both of which may result in an advanced evasion advantage from host defenses and antimicrobial treatment. First identified in S. epidermidis, polysaccharide extracellular adhesin (PIA) is a secreted factor important for intercellular adhesion and aggregation in Staphylococcus (136-138). In addition to polysaccharide-based aggregation, overexpression of certain cell wall anchored proteins can also drive aggregation (139). In S. aureus, Resch et al. identified genes encoding PIA, peptidoglycan modeling factors, and clumping factors were significantly over-expressed during aggregation and early biofilm attachment(140). Though the majority of research seeks to understand how these cell wall anchored proteins contribute to virulence, recent evidence suggests that loss or inactivation of membrane proteins can reduce cell membrane stability, inhibit toxin secretion, and increase susceptibility to the human innate immune system(141).

Periplasmic adaptor proteins (PAPs) are relatively understudied versatile proteins that actively contribute to the architecture of tripartite efflux assemblies, namely multidrug efflux pumps and type I secretion systems, and play diverse roles in the transport process. Recognition between the PAPs and outer membrane factors is essential for pump assembly and function, and targeting this interaction may provide a novel avenue for combating multidrug resistance(142). Several pump proteins that acquired missense mutations in our evolution experiment belong to this category including major facilitator superfamily (MFS) pumps, MdeA and MepA, the ATP-binding cassette ABC superfamily pump, HlyD, and the resistance-nodulation-cell division (RND) pump, FarR. Firstly, MdeA and MepA are both chromosomally encoded MDR efflux pumps(143). MepR is a multidrug binding transcription regulator that represses expression of mepA, as well as its own gene. However, nonsense mutations identified in mepR at residue E72 likely truncated this protein and abolished transcriptional repression of mepA (FIG. 13M). Additionally, mutations identified-84 bp upstream mdeA might also contribute to increased mdeA transcription and subsequent pump activity. Point mutations previously identified at this position were mapped to the −35-promoter consensus sequence of S. aureus strain QM-RM8 and prompted mdeA overexpression and increased resistance to a variety of MdeA substrates(144). Interestingly, S. aureus intra clonal competition has been proposed as a mechanism capable of driving antibiotic resistance evolution(97). Given mdeA mutations were only identified in control planktonic lineages, future work is needed to elucidate the MdeA pump's role in intra clonal competition in S. aureus 29213.

Unlike MFS pumps that use the proton motive force to drive the extrusion of their substrates, ABC superfamily transporters use ATP to drive the extrusion of their substrates. ABC transporters, also known as traffic ATPases, frequently extend beyond the cell membrane into the environment and are responsible for the translocation of a variety of compounds across prokaryotic membranes(145,146). Eighty percent of CFE exposed lineages had new junction evidence on the circular 4.69 Kb plasmid between hlyD) and an ATP binding cassette domain containing protein (FIG. 14). Previous bioinformatic analyses performed by Diebert and colleagues showed that if hlyD is present in Staphylococcus species, it is always located in an operon with two other genes—each encoding for a putative ABC transport system ATP-binding protein and an ABC transport system permease protein(147). Each possesses its own start codon, but they share one Shine-Dalgarno sequence and one promotor region. Interestingly, this group also deduced the ATP-binding protein and the ABC transport permease protein are structurally similar to E. coli FtsE and FtsX, respectively(147). So far, there is no function described for the S. aureus HlyD, FtsE and FtsX proteins, however recent evidence suggests mutations in FtsK, a membrane protein requiring proper FtsEX assembly and recruitment(148), reduced S. aureus cytotoxicity in a T2 cell line infection model(149). In our experiments, the location of this junction yielded frameshift mutations in the FtsE start codon and truncated the 331^{st} residue of the HlyD structure (FIG. 13M). Taken together, it is plausible this junction contributed to the reduced toxicity observed in CFE evolved lineages (FIGS. 12A-12B). Interestingly, recent work in Staphylococcus carnosus discovered that the HlyD, FtsE and FtsX protein complex interacts with penicillin-binding protein 2 and allows this species to adapt to various conditions by modification of their peptidoglycan(147). In line with these findings, our analyses identified penicillin binding protein missense mutations only in lineages where this new junction evidence was identified (FIG. 13M). Therefore, future studies will seek to elucidate the interactions between the HlyD, FtsE and FtsX protein complex and peptidoglycan modeling in S. aureus. Even though peptidoglycan is virtually universal in bacteria, there is a notable degree of diversity in its chemical structure, and modifications in both the sugars and peptides are known to be instrumental for bacteria to cope with diverse environmental challenges(150).

ATP dependent Mur ligases (MurC-F) catalyze the early steps of bacterial peptidoglycan (PG) biosynthesis and are under-exploited targets for antibacterial drug design(151,152). Each Mur ligase possesses specific advantages as an individual antibacterial target, and the four enzymes share conserved features that can be exploited for the design of inhibitors that simultaneously target them all. However, MurE is the only Mur ligase with a substrate specificity that varies among bacteria. Gram-positive bacteria like B. subtilis and S. aureus contain a L-Lys residue at the third position of the PG peptide moiety rather than a meso-diaminopimelic acid (mA2 pm) residue employed by gram negative species. The incorporation of the correct species-specific amino acid substrate by MurE is critical as the third residue of the PG peptide moiety is involved in glycan strand cross-linkages and thus plays a key role in maintaining PG integrity(153). Addition of the incorrect amino acid at the third position of the PG peptide moiety can yield morphological changes and bacterial cell lysis(154, 155). Here, mutations affecting MurE occurred primarily at 1464 (FIG. 13M) and predominantly in Bs CFE and control biofilm evolved lineages (FIG. 14). Previous work by Ruane et al demonstrated that L-lysine's inability to bind the 1464 residue impairs structural stability in the active site, inhibits the L-lysine-binding activity required of functional MurE (124), and leads to increased peptidoglycan recycling and turnover. Peptidoglycan hydrolases help to remodel and recycle peptidoglycan, and these processes are known to drive bacterial aggregation and biofilm formation(156). As demonstrated above, B. subtilis 6D1 CFE upregulates agr, RNAIII, and saeR in S. aureus ATCC 29213, and overexpression of these regulators also increases transcription of peptidoglycan hydrolases lytM, ssaA, sceD(157). Based on these data, it is probable that mutations in murE 1464 identified in our study may have contributed to peptidoglycan modifications that increased aggregation, however, future studies are needed to thoroughly investigate this relationship and deduce which molecule(s) in B. subtilis 6D1 CFE drove the evolution of this phenotype.

In addition to inhibiting aggregation and biofilm formation, anti-virulence therapies that block S. aureus toxin production have shown potential to also thwart bacteria's acquisition of antibiotic resistance(158). Expression and secretion of these virulence factors are tightly controlled by multiple regulatory systems, however almost all rely on functional RNA polymerases(159). RNA polymerase (RNAP) is the key enzyme responsible for transcription of DNA into RNA, and natural products produced by other bacteria have already demonstrated that it's possible to inhibit RNA polymerase function in S. aureus (160). Bacterial RNAP core enzyme consists of several subunits: the α (rpoA) dimer that holds together β (rpoB) and β′ (rpoC), form the catalytic center, and the ω (rpoZ) subunit that binds to β′. This core enzyme, α2ββ′ω, is conserved across the bacterial kingdom, however members of the Firmicutes contain an additional subunit, δ (rpoE), which is also bound to β′ subunit(161). The δ subunit is essential for cell survival when facing a competing strain in a changing environment, and although δ is not essential per se, it is vital for the cell's ability to rapidly adapt and survive in nature(127). In fact, S. aureus mutants where δ or ω fail to bind to the β′ subunit have diminished toxin activity and abundance, are less virulent in a murine model of infection(162), and harbor an impaired ability to resist stress and maintain biofilm structure(163). In our study, rpoC mutations were found in ⅗ Bs CFE biofilm populations and ⅗ control planktonic populations (FIG. 14). Interestingly, both treatments displayed significant competitive defects compared to the ancestor (FIGS. 11A-11B). Alterations in RNAP function also plays an important role in the development of antibiotic resistance. In addition to binding the virulence critical δ subunit, mutations in rpoC at residue P440 augment cell wall peptidoglycan synthesis and create a slow vancomycin intermediate S. aureus (VISA) phenotype (164,165). Mutations identified in our lineages occurred at residues N254Y, L263F and D264V, however, satellite colonies were identified in four different lineages after 72 hr incubation (FIG. 13L). Despite increased phenotypic sensitivity in multiple lineages, satellite colonies were also observed in lineages spanning all evolutionary conditions after treatment with Rifampin (FIG. 13L). Rifampin is an RNAP inhibitor that binds to the β subunit of RNAP and blocks the elongation of RNA(166). Though rpoB mutations conferring susceptibility to Rifampin have been identified(167), our study suggests mutations in rpoA and rpoC, might also play a role in Rifampin susceptibility in S. aureus (FIG. 13C). However, further research is needed to determine how these mutations affect RNAP ribosomal binding and transcription(168). The role of FdhD in virulence and antibiotic sensitivity is less clear, although any changes to the metabolism of a cell are likely to have significant downstream effects on gene expression(169). Specifically, work by Lei et al discovered fdhD is downregulated by rsp, a regulatory protein that represses biofilm formation by affecting production of surface proteins(170). Though these mutations were found almost exclusively in control lineages (FIG. 14), it is tempting to speculate that these mutations conferred fitness defects in MHB media (FIG. 13K). More research is needed to elucidate the molecular detail of how these proteins affect virulence under different metabolic conditions.

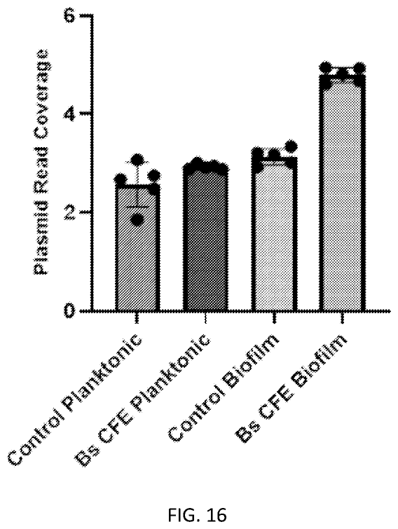

B. subtilis 6D1 CFE does not kill S. aureus (FIG. 9A), and since quorum sensing interference is not believed to induce strong selection pressure, it is possible the increased phenotypic sensitivity and decreased virulence observed in the Bs CFE-evolved populations is due to the fitness cost of harboring excess plasmid replicons or the benefit of losing plasmid borne resistance traits(171). However, higher mean coverage depth was observed in reads mapped to the 4.69 Kb plasmid in CFE evolved lineages compared to their control counterparts (FIG. 16). Furthermore, mutations in plasmid associated genes were found more frequently and in higher abundance in CFE exposed lineages (FIG. 14), suggesting a loss in plasmid borne traits is unlikely driving these phenotypes in CFE evolved S. aureus lineages. However, lower plasmid read coverage experienced by control populations may help to explain the fitness deficiencies observed during planktonic competition against B. subtilis 6D1 (FIG. 10A). It is possible this plasmid plays an important role in mediating fitness in response to environmental stressors. In fact, RacA, the gene purported to transcribe DUF536 is significantly correlated with gut-associated S. epidermidis isolates(172), and loss of the plasmid harboring racA rendered a clinical MRSA strain Agr defective, but hypervirulent(173). In line with these findings, work by Laabei and colleagues found that S. aureus strains that caused the most severe disease were the least toxic due, in part, to within-host fitness differences(149). Despite previous clinical trials showing a reduction in S. aureus colonization after supplementation with a B. subtilis probiotic(79), future studies should aim to investigate how residual S. aureus populations evolve in these host associated systems. Nevertheless, these data strengthen the notion that probiotic derived compounds harboring QSI activity, like those found it B. subtilis 6D1 CFE, can maintain antibiofilm activity after ~80 generations without increasing cross resistance to antibiotics targeting a myriad of vital physiological processes.

In describing the present invention and its various embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention.

Various patents and other publications are referred to throughout the specification. Each of these publications is incorporated by reference herein as if each had been individually incorporated, in its entirety.

### REFERENCES

1. Nelson R E, Hatfield K M, Wolford H, Samore M H, Scott R D II, Reddy S C, et al. National Estimates of Healthcare Costs Associated With Multidrug-Resistant Bacterial Infections Among Hospitalized Patients in the United States. Clin Infect Dis. 2021 Jan. 15; 72(Supplement_1):S17-26.
2 Rose W E, Poppens P T. Impact of biofilm on the in vitro activity of vancomycin alone and in combination with tigecycline and rifampicin against Staphylococcus aureus. J Antimicrob Chemother. 2009 Mar. 1; 63(3):485-8.
3 Savage V J, Chopra I, O'Neill A J. Staphylococcus aureus Biofilms Promote Horizontal Transfer of Antibiotic Resistance. Antimicrob Agents Chemother. 2013 April; 57(4):1968-70.
4. Høiby N, Bjarnsholt T, Givskov M, Molin S, Ciofu O. Antibiotic resistance of bacterial biofilms. Int J Antimicrob Agents. 2010 Apr. 1; 35(4):322-32.
5. Seerengeraj V. Probiotics: The Marvelous Factor and Health Benefits. Biomed Biotechnol Res J BBRJ. 2018 Mar. 8; 2:1-8.
6. Landini P, Antoniani D, Burgess J G, Nijland R. Molecular mechanisms of compounds affecting bacterial biofilm formation and dispersal. Appl Microbiol Biotechnol. 2010 April; 86(3):813-23.
7. Ahmad M, Khan A U. Global economic impact of antibiotic resistance: A review. J Glob Antimicrob Resist. 2019; 19:313-6.
8. Centers for Disease Control and Prevention (U.S.). Antibiotic resistance threats in the United States, 2019 [Internet]. Centers for Disease Control and Prevention (U.S.); 2019 November [cited 2020 Mar. 29]. Available from: https://stacks.cdc.gov/view/cdc/82532
9. Ghosh C, Sarkar P, Issa R, Haldar J. Alternatives to Conventional Antibiotics in the Era of Antimicrobial Resistance. Trends Microbiol. 2019 April; 27(4):323-38.
10. Varankovich N V, Nickerson M T, Korber D R. Probiotic-based strategies for therapeutic and prophylactic use against multiple gastrointestinal diseases. Front Microbiol [Internet]. 2015 Jul. 14 [cited 2020 Mar. 21]; 6. Available from: http://journal.frontiersin.org/Article/10.3389/fmicb.2015.00685/abstract.
11. Plaza-Diaz J, Ruiz-Ojeda F J, Gil-Campos M, Gil A. Mechanisms of Action of Probiotics. Adv Nutr. 2019 January; 10(Suppl 1):S49-66.
12. Zhou L, Zhang Y, Ge Y, Zhu X, Pan J. Regulatory Mechanisms and Promising Applications of Quorum Sensing-Inhibiting Agents in Control of Bacterial Biofilm Formation. Front Microbiol. 2020 Oct. 15; 11:589640.
13. Abraham W R. Going beyond the Control of Quorum-Sensing to Combat Biofilm Infections. Antibiotics. 2016 Jan. 9; 5(1):3.
14. Bosi E, Monk J M, Aziz R K, Fondi M, Nizet V, Palsson BØ. Comparative genome-scale modelling of Staphylococcus aureus strains identifies strain-specific metabolic capabilities linked to pathogenicity. Proc Natl Acad Sci. 2016 Jun. 28; 113(26):E3801-9.
15. Silva K P T, Boedicker J Q. A neural network model predicts community-level signaling states in a diverse microbial community. PLOS Comput Biol. 2019 Jun. 24; 15(6):e1007166.
16. Ji G, Beavis R, Novick R P. Bacterial interference caused by autoinducing peptide variants. Science. 1997 Jun. 27; 276(5321):2027-30.
17. Tan L, Li S R, Jiang B, Hu X M, Li S. Therapeutic Targeting of the Staphylococcus aureus Accessory Gene Regulator (agr) System. Front Microbiol. 2018 Jan. 25; 9:55.
18. Özcan S S, Dieser M, Parker A E, Balasubramanian N, Foreman C M. Quorum sensing inhibition as a promising method to control biofilm growth in metalworking fluids. J Ind Microbiol Biotechnol. 2019 August; 46(8):1103-11.
19. Cheng W J, Zhou J W, Zhang P P, Luo H Z, Tang S, Li J J, et al. Quorum sensing inhibition and tobramycin acceleration in Chromobacterium violaceum by two natural cinnamic acid derivatives. Appl Microbiol Biotechnol. 2020 June; 104(11):5025-37.
20. Tong S Y C, Chen L F, Fowler V G. Colonization, pathogenicity, host susceptibility, and therapeutics for Staphylococcus aureus: what is the clinical relevance? Semin Immunopathol. 2012 March; 34(2):185-200.
21. Loughman J A, Fritz S A, Storch G A, Hunstad D A. Virulence gene expression in human community-acquired Staphylococcus aureus infection. J Infect Dis. 2009 Feb. 1; 199(3):294-301.
22. Novick R P, Geisinger E. Quorum Sensing in Staphylococci. Annu Rev Genet. 2008 December; 42(1):541-64.
23. Sakoulas G, Eliopoulos G M, Moellering R C, Wennersten C, Venkataraman L, Novick R P, et al. Accessory gene regulator (agr) locus in geographically diverse Staphylococcus aureus isolates with reduced susceptibility to vancomycin. Antimicrob Agents Chemother. 2002 May; 46(5):1492-502.
24. Jarraud S, Mougel C, Thioulouse J, Lina G, Meugnier H, Forey F, et al. Relationships between Staphylococcus aureus genetic background, virulence factors, agr groups (alleles), and human disease. Infect Immun. 2002 February; 70(2):631-41.
25. Galia L, Ligozzi M, Bertoncelli A, Mazzariol A, Department of Diagnostics and Public Health, University of Verona, Verona, Italy. Real-time PCR assay for detection of Staphylococcus aureus, Panton-Valentine Leucocidin and Methicillin Resistance directly from clinical samples. AIMS Microbiol. 2019; 5(2):138-46.
26. Yarwood J M, Schlievert P M. Quorum sensing in Staphylococcus infections. J Clin Invest. 2003 Dec. 1; 112(11):1620-5.
27. Yarwood J M, Bartels D J, Volper E M, Greenberg E P. Quorum sensing in Staphylococcus aureus biofilms. J Bacteriol. 2004 March; 186(6):1838-50.
28. Gangwar B, Kumar S, Darokar M P. Glabridin Averts Biofilms Formation in Methicillin-Resistant Staphylococcus aureus by Modulation of the Surfaceome. Front Microbiol. 2020 Sep. 17; 11:1779.
29. Vollaro A, Esposito A, Esposito E P, Zarrilli R, Guaragna A, De Gregorio E. PYED-1 Inhibits Biofilm Formation and Disrupts the Preformed Biofilm of Staphylococcus aureus. Antibiotics. 2020 May 8; 9(5):240.
30. Liu Y, She P, Xu L, Chen L, Li Y, Liu S, et al. Antimicrobial, Antibiofilm, and Anti-persister Activities of Penfluridol Against Staphylococcus aureus. Front Microbiol. 2021 Aug. 18; 12:727692.
31. Sánchez B, Delgado S, Blanco-Míguez A, Lourenço A, Gueimonde M, Margolles A. Probiotics, gut microbiota, and their influence on host health and disease. Mol Nutr Food Res. 2017; 61(1).
32. Warrack S R, Panjikar P, Duster M, Safdar N. Probiotics for the Reduction of Colonization with MRSA. Am J Infect Control. 2013 June; 41(6):S135-6.
33. Luo G, Li B, Yang C, Wang Y, Bian X, Li W, et al. Major Traditional Probiotics: Comparative Genomic Analyses and Roles in Gut Microbiome of Eight Cohorts. Front Microbiol [Internet]. 2019 [cited 2021 Mar. 19]; 10. Available from: https://www.frontiersin.org/articles/10.3389/fmicb.2019.00712/full
34. Wieërs G, Belkhir L, Enaud R, Leclercq S, Philippart de Foy J M, Dequenne I, et al. How Probiotics Affect the Microbiota. Front Cell Infect Microbiol [Internet]. 2020 [cited 2020 Dec. 6]; 9. Available from: https://www.frontiersin.org/articles/10.3389/fcimb.2019.00454/full.
35. Land M, Hauser L, Jun S R, Nookaew I, Leuze M R, Ahn T H, et al. Insights from 20 years of bacterial genome sequencing. Funct Integr Genomics. 2015; 15(2):141-61.
36. Earl A M, Losick R, Kolter R. Ecology and genomics of Bacillus subtilis. Trends Microbiol. 2008 June; 16(6):269-75.
37. Earl A M, Eppinger M, Fricke W F, Rosovitz M J, Rasko D A, Daugherty S, et al. Whole-genome sequences of Bacillus subtilis and close relatives. J Bacteriol. 2012 May; 194(9):2378-9.
38. Steinke K, Mohite O S, Weber T, Kovács A T. Phylogenetic Distribution of Secondary Metabolites in the Bacillus subtilis Species Complex. mSystems [Internet]. 2021 Apr. 27 [cited 2021 May 6]; 6(2). Available from: https://msystems.asm.org/content/6/2/e00057-21
39. Kiesewalter H T, Lozano-Andrade C N, Wibowo M, Strube M L, Maróti G, Snyder D, et al. Genomic and chemical diversity of Bacillus subtilis secondary metabolites against plant pathogenic fungi. bioRxiv. 2020 Aug. 5; 2020.08.05.238063.
40. Caulier S, Nannan C, Gillis A, Licciardi F, Bragard C, Mahillon J. Overview of the Antimicrobial Compounds Produced by Members of the Bacillus subtilis Group. Front Microbiol. 2019 Feb. 26; 10:302.
41. Kimelman H, Shemesh M. Probiotic Bifunctionality of Bacillus subtilis-Rescuing Lactic Acid Bacteria from Desiccation and Antagonizing Pathogenic Staphylococcus aureus. Microorganisms. 2019 Sep. 29; 7(10).
42. Qiu M, Feng L, Zhao C, Gao S, Bao L, Zhao Y, et al. Commensal Bacillus subtilis from cow milk inhibits Staphylococcus aureus biofilm formation and mastitis in mice. FEMS Microbiol Ecol. 2022 Jun. 1; fiac065.
43. Balouiri M, Sadiki M, Ibnsouda S K. Methods for in vitro evaluating antimicrobial activity: A review. J Pharm Anal. 2016 April; 6(2):71-9.
44. Yang I C, Shih DYC, Huang T P, Huang Y P, Wang J Y, Pan™. Establishment of a novel multiplex PCR assay and detection of toxigenic strains of the species in the Bacillus cereus group. J Food Prot. 2005 October; 68(10):2123-30.
45. Scribner M R, Santos-Lopez A, Marshall C W, Deitrick C, Cooper V S. Parallel evolution of tobramycin resistance across species and environments [Internet]. Microbiology; 2019 September [cited 2020 Apr. 1]. Available from: http://biorxiv.org/lookup/doi/10.1101/758979
46. Turner C B, Marshall C W, Cooper V S. Parallel genetic adaptation across environments differing in mode of growth or resource availability. Evol Lett. 2018; 2(4):355-67.
47. Santos-Lopez A, Marshall C W, Scribner M R, Snyder D J, Cooper V S. Evolutionary pathways to antibiotic resistance are dependent upon environmental structure and bacterial lifestyle. eLife. 2019 Sep. 13; 8: e47612.
48. O'Neill E, Pozzi C, Houston P, Humphreys H, Robinson D A, Loughman A, et al. A Novel Staphylococcus aureus Biofilm Phenotype Mediated by the Fibronectin-Binding Proteins, FnBPA and FnBPB. J Bacteriol. 2008 June; 190(11):3835-50.
49. Travisano M, Lenski R E. Long-Term Experimental Evolution in Escherichia coli. IV. Targets of Selection and the Specificity of Adaptation.:12.
50. Humphries R M, Abbott A N, Hindler J A. Understanding and Addressing CLSI Breakpoint Revisions: a Primer for Clinical Laboratories. Kraft C S, editor. J Clin Microbiol. 2019 Apr. 10; 57(6): e00203-19,/jcm/57/6/JCM.00203-19.atom.
51. bcl2fastq2 Conversion Software v2.20 Software Guide (15051736).:27.
52. Wick R. rrwick/Porechop [Internet]. 2021 [cited 2021 May 6]. Available from: https://github.com/rrwick/Porechop
53. Wick R R, Judd L M, Gorrie C L, Holt K E. Unicycler: Resolving bacterial genome assemblies from short and long sequencing reads. PLOS Comput Biol. 2017 June; 13(6): e1005595.
54. Gurevich A, Saveliev V, Vyahhi N, Tesler G. QUAST: quality assessment tool for genome assemblies. Bioinforma Oxf Engl. 2013 Apr. 15; 29(8):1072-5.
55. Seemann T. Prokka: rapid prokaryotic genome annotation. Bioinforma Oxf Engl. 2014 Jul. 15; 30(14):2068-9.
56. Blin K, Shaw S, Kloosterman A M, Charlop-Powers Z, van Wezel G P, Medema M H, et al. antiSMASH 6.0: improving cluster detection and comparison capabilities. Nucleic Acids Res. 2021 Jul. 2; 49 (W1): W29-35.
57. Gilchrist CLM, Chooi Y H. Clinker & clustermap.js: Automatic generation of gene cluster comparison figures. Bioinforma Oxf Engl. 2021 Jan. 18; btab007.
58. Dimkić I, Stanković S, Nišavić M, Petković M, Ristivojević P, Fira D, et al. The Profile and Antimicrobial Activity of Bacillus Lipopeptide Extracts of Five Potential Biocontrol Strains. Front Microbiol. 2017; 8:925.
59. Harwood C R, Mouillon J M, Pohl S, Arnau J. Secondary metabolite production and the safety of industrially important members of the Bacillus subtilis group. FEMS Microbiol Rev. 2018 Nov. 1; 42(6):721-38.
60. Sharma D, Singh S S, Baindara P, Sharma S, Khatri N, Grover V, et al. Surfactin Like Broad Spectrum Antimicrobial Lipopeptide Co-produced With Sublancin From Bacillus subtilis Strain A52: Dual Reservoir of Bioactives. Front Microbiol. 2020 Jun. 11; 11:1167.
61. Vincenti F, Montesano C, Di Ottavio F, Gregori A, Compagnone D, Sergi M, et al. Molecular Networking: A Useful Tool for the Identification of New Psychoactive Substances in Seizures by LC-HRMS. Front Chem. 2020; 8:1039.
62. Beltrame C O, Cortes M F, Bandeira P T, Figueiredo A M S. Optimization of the RNeasy Mini Kit to obtain high-quality total RNA from sessile cells of Staphylococcus aureus. Braz J Med Biol Res Rev Bras Pesqui Medicas E Biol. 2015 December; 48(12):1071-6.
63. Canovas J, Baldry M, Bojer M S, Andersen P S, Gless B H, Grzeskowiak P K, et al. Cross-Talk between Staphylococcus aureus and Other Staphylococcal Species via the agr Quorum Sensing System. Front Microbiol. 2016; 7:1733.
64. Livak K J, Schmittgen T D. Analysis of relative gene expression data using real-time quantitative PCR and the 2(-Delta Delta C(T)) Method. Methods San Diego Calif. 2001 December; 25(4):402-8.
65. Monnappa A K, Bari W, Choi S Y, Mitchell R J. Investigating the Responses of Human Epithelial Cells to Predatory Bacteria. Sci Rep. 2016 Sep. 15; 6(1):33485.
66. Kornienko M A, Kopyltsov V N, Shevlyagina N V, Didenko L V, Lyubasovskaya L A, Priputnevich T V, et al. [ABILITY O F STAPHYLOCOCCUS O F VARIOUS STRAINS T O CREATE BIOFILMS AND THEIR EFFECT O N HUMAN BODY CELLS]. Mol Genet Mikrobiol Virusol. 2016; 34(1):18-25.
67. Moormeier D E, Bose J L, Horswill A R, Bayles K W. Temporal and Stochastic Control of Staphylococcus aureus Biofilm Development. Dunny G M, Greenberg E P, editors. mBio. 2014 Oct. 14; 5(5): e01341-14.
68. Vuong C, Saenz H L, Götz F, Otto M. Impact of the agr Quorum-Sensing System on Adherence to Polystyrene in Staphylococcus aureus. J Infect Dis. 2000 Dec. 1; 182(6):1688-93.
69. Liu Q, Yeo W S, Bae T. The SaeRS Two-Component System of Staphylococcus aureus. Genes. 2016 Oct. 3; 7(10):81.
70. Burton A T, Kearns D B. The Large pBS32/pLS32 Plasmid of Ancestral Bacillus subtilis. J Bacteriol. 2020 Aug. 25; 202(18): e00290-20.
71. Tanaka T, Koshikawa T. Isolation and characterization of four types of plasmids from Bacillus subtilis (natto). J Bacteriol. 1977 August; 131(2):699-701.
72. Piewngam P, Zheng Y, Nguyen T H, Dickey S W, Joo H S, Villaruz A E, et al. Pathogen elimination by probiotic Bacillus via signalling interference. Nature. 2018 October; 562(7728):532-7.
73. Liu J, Li W, Zhu X, Zhao H, Lu Y, Zhang C, et al. Surfactin effectively inhibits Staphylococcus aureus adhesion and biofilm formation on surfaces. Appl Microbiol Biotechnol. 2019 June; 103(11):4565-74.
74. Vahidinasab M, Lilge L, Reinfurt A, Pfannstiel J, Henkel M, Morabbi Heravi K, et al. Construction and description of a constitutive plipastatin mono-producing Bacillus subtilis. Microb Cell Factories. 2020 Nov. 10; 19(1):205.
75. Kranjec C, Morales Angeles D, Torrissen Mårli M, Fernández L, García P, Kjos M, et al. Staphylococcal Biofilms: Challenges and Novel Therapeutic Perspectives. Antibiotics. 2021 Jan. 29; 10(2):131.
76. Otto M. Staphylococcal Biofilms. Microbiol Spectr. 2018 Aug. 16; 6(4):6.4.27.
77. Rémy B, Mion S, Plener L, Elias M, Chabrière E, Daudé D. Interference in Bacterial Quorum Sensing: A Biopharmaceutical Perspective. Front Pharmacol. 2018; 9:203.
78. Gonzalez D, Sabnis A, Foster K R, Mavridou DAI. Costs and benefits of provocation in bacterial warfare. Proc Natl Acad Sci. 2018 Jul. 17; 115(29):7593-8.
79. Piewngam P, Khongthong S, Roekngam N, Theapparat Y, Sunpaweravong S, Faroongsarng D, et al. Probiotic for pathogen-specific Staphylococcus aureus decolonisation in Thailand: a phase 2, double-blind, randomised, placebo-controlled trial. Lancet Microbe [Internet]. 2023 Jan. 13 [cited 2023 Jan. 16]; 0(0). Available from: https://www.thelancet.com/journals/lanmic/article/PIIS2666-5247(22) 00322-6/fulltext.
80. Zhang F, Wang B, Liu S, Chen Y, Lin Y, Liu Z, et al. Bacillus subtilis revives conventional antibiotics against Staphylococcus aureus osteomyelitis. Microb Cell Factories. 2021 May 17; 20(1):102.
81. Paulander W, Varming A N, Bojer M S, Friberg C, Bæk K, Ingmer H. The agr quorum sensing system in Staphylococcus aureus cells mediates death of sub-population. BMC Res Notes. 2018 Jul. 24; 11:503.
82. Voyich J M, Vuong C, DeWald M, Nygaard T K, Kocianova S, Griffith S, et al. The SaeR/S Gene Regulatory System is Essential for Innate Immune Evasion by Staphylococcus aureus. J Infect Dis. 2009 Jun. 1; 199(11):1698-706.
83. Tuchscherr L, Bischoff M, Lattar S M, Noto Llana M, Pförtner H, Niemann S, et al. Sigma Factor SigB Is Crucial to Mediate Staphylococcus aureus Adaptation during Chronic Infections. PLOS Pathog. 2015 April; 11(4): e1004870.
84. Boles B R, Horswill A R. agr-Mediated Dispersal of Staphylococcus aureus Biofilms. PLOS Pathog. 2008 Apr. 25; 4(4): e1000052.
85. Zapotoczna M, McCarthy H, Rudkin J K, O'Gara J P, O'Neill E. An Essential Role for Coagulase in Staphylococcus aureus Biofilm Development Reveals New Therapeutic Possibilities for Device-Related Infections. J Infect Dis. 2015 Dec. 15; 212(12):1883-93.
86. Lauderdale K J, Boles B R, Cheung A L, Horswill A R. Interconnections between Sigma B, agr, and proteolytic activity in Staphylococcus aureus biofilm maturation. Infect Immun. 2009 April; 77(4):1623-35.
87. Olson M E, Todd D A, Schaeffer C R, Paharik A E, Van Dyke M J, Büttner H, et al. Staphylococcus epidermidis agr Quorum-Sensing System: Signal Identification, Cross Talk, and Importance in Colonization. J Bacteriol. 2014 October; 196(19):3482-93.
88. Yang T, Tal-Gan Y, Paharik A E, Horswill A R, Blackwell H E. Structure-Function Analyses of a Staphylococcus epidermidis Autoinducing Peptide Reveals Motifs Critical for AgrC-type Receptor Modulation. ACS Chem Biol. 2016 Jul. 15; 11(7):1982-91.
89. Dempwolff F, Sanchez S, Kearns D B. TnFLX: a Third-Generation mariner-Based Transposon System for Bacillus subtilis. Appl Environ Microbiol. 2020 May 5; 86(10): e02893-19.
90. Konkol M A, Blair K M, Kearns D B. Plasmid-Encoded ComI Inhibits Competence in the Ancestral 3610 Strain of Bacillus subtilis. J Bacteriol. 2013 Sep. 15; 195(18):4085-93.
91. Villena J, Vizoso-Pinto M G, Kitazawa H. Intestinal Innate Antiviral Immunity and Immunobiotics: Beneficial Effects against Rotavirus Infection. Front Immunol. 2016; 7:563.
92. Iida H, Tohno M, Islam M A, Sato N, Kobayashi H, Albarracin L, et al. Paraimmunobiotic Bifidobacteria Modulate the Expression Patterns of Peptidoglycan Recognition Proteins in Porcine Intestinal Epitheliocytes and Antigen Presenting Cells. Cells. 2019 Aug. 14; 8(8): E891.
93. Martínez-Maqueda D, Miralles B, Recio I. HT29 Cell Line. In: Verhoeckx K, Cotter P, López-Expósito I, Kleiveland C, Lea T, Mackie A, et al., editors. The Impact of Food Bioactives on Health: in vitro and ex vivo models [Internet]. Cham: Springer International Publishing; 2015 [cited 2022 September
15]. p. 113-24. Available from: https://doi.org/10.1007/978-3-319-16104-4 11.
94. Yan S, Wu G. Can Biofilm Be Reversed Through Quorum Sensing in Pseudomonas aeruginosa? Front Microbiol. 2019 Jul. 23; 10:1582.
95. Zhao X, Yu Z, Ding T. Quorum-Sensing Regulation of Antimicrobial Resistance in Bacteria. Microorganisms. 2020 Mar. 17; 8(3):425.
96. García-Contreras R, Maeda T, Wood T K. Can resistance against quorum-sensing interference be selected? ISME J. 2016 January; 10(1):4-10.
97. Koch G, Yepes A, Förstner K U, Wermser C, Stengel S T, Modamio J, et al. Evolution of Resistance to a Last-Resort Antibiotic in Staphylococcus aureus via Bacterial Competition. Cell. 2014 August; 158(5):1060-71.
98. Dillon M M, Sung W, Sebra R, Lynch M, Cooper V S. Genome-Wide Biases in the Rate and Molecular Spectrum of Spontaneous Mutations in Vibrio cholerae and Vibrio fischeri. Molecular Biology and Evolution. 2017 Jan. 1; 34(1):93-109.
99. Lynch M, Ackerman M S, Gout J F, Long H, Sung W, Thomas W K, et al. Genetic drift, selection and the evolution of the mutation rate. Nat Rev Genet. 2016 Oct. 14; 17(11):704-14.
100. Kowalska-Krochmal B, Dudek-Wicher R. The Minimum Inhibitory Concentration of Antibiotics: Methods, Interpretation, Clinical Relevance. Pathogens. 2021 Feb. 4; 10(2):165.
101. Zhao J, Carmody L A, Kalikin L M, Li J, Petrosino J F, Schloss P D, et al. Impact of Enhanced Staphylococcus DNA Extraction on Microbial Community Measures in Cystic Fibrosis Sputum. PLOS One. 2012 Mar. 8; 7(3): e33127.
102. Soni I, Chakrapani H, Chopra S. Draft Genome Sequence of Methicillin-Sensitive Staphylococcus aureus ATCC 29213. Genome Announcements. 3(5): e01095-15.
103. Deatherage D E, Barrick J E. Identification of mutations in laboratory evolved microbes from next-generation sequencing data using breseq. Methods Mol Biol. 2014; 1151:165-88.
104. Roberts P H, Davis K C, Garstka W R, Bhunia A K. Lactate dehydrogenase release assay from Vero cells to distinguish verotoxin producing Escherichia coli from non-verotoxin producing strains. J Microbiol Methods. 2001 January; 43(3):171-81.
105. Vogwill T, MacLean R C. The genetic basis of the fitness costs of antimicrobial resistance: a meta-analysis approach. Evol Appl. 2015 March; 8(3):284-95.
106. Sonesson A, Przybyszewska K, Eriksson S, Mörgelin M, Kjellström S, Davies J, et al. Identification of bacterial biofilm and the Staphylococcus aureus derived protease, staphopain, on the skin surface of patients with atopic dermatitis. Sci Rep. 2017 Aug. 18; 7:8689.
107. Di Domenico E G, Cavallo I, Capitanio B, Ascenzioni F, Pimpinelli F, Morrone A, et al. Staphylococcus aureus and the Cutaneous Microbiota Biofilms in the Pathogenesis of Atopic Dermatitis. Microorganisms. 2019 Aug. 29; 7(9):301.
108. Algburi A, Al-Hasani H M, Ismael T K, Abdelhameed A, Weeks R, Ermakov A M, et al. Antimicrobial Activity of Bacillus subtilis KATMIRA1933 and Bacillus amyloliquefaciens B-1895 Against Staphylococcus aureus Biofilms Isolated from Wound Infection. Probiotics & Antimicro Prot. 2021 February; 13(1):125-34.
109. Park S, Ronholm J. Staphylococcus aureus in Agriculture: Lessons in Evolution from a Multispecies Pathogen. Clin Microbiol Rev. 2021 Feb. 10; 34(2): e00182-20.
110. Young B C, Golubchik T, Batty E M, Fung R, Larner-Svensson H, Votintseva A A, et al. Evolutionary dynamics of Staphylococcus aureus during progression from carriage to disease. Proc Natl Acad Sci USA. 2012 Mar. 20; 109(12):4550-5.
111. Mellbye B, Schuster M. The sociomicrobiology of antivirulence drug resistance: a proof of concept. mBio. 2011; 2(5): e00131-11.
112. Stefanic P, Belcijan K, Kraigher B, Kostanjšek R, Nesme J, Madsen J S, et al. Kin discrimination promotes horizontal gene transfer between unrelated strains in Bacillus subtilis. Nat Commun. 2021 Jun. 8; 12(1):3457.
113. Maan H, Itkin M, Malitsky S, Friedman J, Kolodkin-Gal I. Resolving the conflict between antibiotic production and rapid growth by recognition of peptidoglycan of susceptible competitors. Nat Commun. 2022 Jan. 20; 13(1):431.
114. Nair R R, Andersson D I. Interspecies interaction reduces selection for antibiotic resistance in Escherichia coli. Commun Biol. 2023 Mar. 27; 6(1):1-9.
115. Lade H, Park J H, Chung S H, Kim I H, Kim J M, Joo H S, et al. Biofilm Formation by Staphylococcus aureus Clinical Isolates is Differentially Affected by Glucose and Sodium Chloride Supplemented Culture Media. Journal of Clinical Medicine. 2019 November; 8(11):1853.
116. Missiakas D M, Schneewind O. Growth and laboratory maintenance of Staphylococcus aureus. Curr Protoc Microbiol. 2013 February; Chapter 9: Unit 9C.1.
117. Trunk T, Khalil H S, Leo J C. Bacterial autoaggregation. AIMS Microbiol. 2018 Mar. 1; 4(1):140-64.
118. Burel C, Dreyfus R, Purevdorj-Gage L. Physical mechanisms driving the reversible aggregation of Staphylococcus aureus and response to antimicrobials. Sci Rep. 2021 Jul. 22; 11(1):15048.
119. Jumper J, Evans R, Pritzel A, Green T, Figurnov M, Ronneberger O, et al. Highly accurate protein structure prediction with AlphaFold. Nature. 2021 August; 596(7873):583-9.
120. Schrödinger, LLC. The PyMOL Molecular Graphics System, Version 1.8. 2015.
121. Haaber J, Cohn M T, Frees D, Andersen T J, Ingmer H. Planktonic Aggregates of Staphylococcus aureus Protect against Common Antibiotics. PLOS ONE. 2012 Jul. 18; 7(7): e41075.
122. Kumaraswami M, Schuman J T, Seo S M, Kaatz G W, Brennan R G. Structural and biochemical characterization of MepR, a multidrug binding transcription regulator of the Staphylococcus aureus multidrug efflux pump MepA. Nucleic Acids Res. 2009 March; 37(4):1211-24.
123. Cordero M, García-Fernández J, Acosta I C, Yepes A, Avendano-Ortiz J, Lisowski C, et al. The induction of natural competence adapts staphylococcal metabolism to infection. Nat Commun. 2022 Mar. 21; 13:1525.
124. Ruane K M, Lloyd A J, Fülöp V, Dowson C G, Barreteau H, Boniface A, et al. Specificity Determinants for Lysine Incorporation in Staphylococcus aureus Peptidoglycan as Revealed by the Structure of a MurE Enzyme Ternary Complex*. Journal of Biological Chemistry. 2013 Nov. 15; 288(46):33439-48.
125. Alnaseri H, Kuiack R C, Ferguson K A, Schneider JET, Heinrichs D E, McGavin M J. DNA Binding and Sensor Specificity of FarR, a Novel TetR Family Regulator Required for Induction of the Fatty Acid Efflux Pump FarE in Staphylococcus aureus. J Bacteriol. 2019 Feb. 1; 201(3): e00602-18.
126. Igarashi K, Ishihama A. Bipartite functional map of the E. coli RNA polymerase alpha subunit: involvement of the C-terminal region in transcription activation by cAMP-CRP. Cell. 1991 Jun. 14; 65(6):1015-22.
127. Rabatinová A, Šanderová H, Jirát Matějčková J, Korelusová J, Sojka L, Barvík I, et al. The δ Subunit of RNA Polymerase Is Required for Rapid Changes in Gene Expression and Competitive Fitness of the Cell. J Bacteriol. 2013 June; 195(11):2603-11.
128. Raghuram V, Alexander A M, Loo H Q, Petit R A, Goldberg J B, Read T D. Species-Wide Phylogenomics of the Staphylococcus aureus Agr Operon Revealed Convergent Evolution of Frameshift Mutations. Microbiology Spectrum. 2022 Jan. 19; 10(1): e01334-21.
129. Bateman A, Coggill P, Finn R D. DUFs: families in search of function. Acta Crystallogr Sect F Struct Biol Cryst Commun. 2010 Mar. 5; 66 (Pt 10):1148-52.
130. Locke J B, Zuill D E, Scharn C R, Deane J, Sahm D F, Denys G A, et al. Linezolid-Resistant Staphylococcus aureus Strain 1128105, the First Known Clinical Isolate Possessing the cfr Multidrug Resistance Gene. Antimicrob Agents Chemother. 2014 November; 58(11):6592-8.
131. Iqbal Z, Seleem M N, Hussain H I, Huang L, Hao H, Yuan Z. Comparative virulence studies and transcriptome analysis of Staphylococcus aureus strains isolated from animals. Sci Rep. 2016 Oct. 14; 6:35442.
132. Crosby H A, Kwiecinski J, Horswill A R. Staphylococcus aureus aggregation and coagulation mechanisms, and their function in host-pathogen interactions. Adv Appl Microbiol. 2016; 96:1-41.
133. Sun F, Zhou L, Zhao B C, Deng X, Cho H, Yi C, et al. Targeting MgrA-mediated virulence regulation in Staphylococcus aureus. Chem Biol. 2011 Aug. 26; 18(8):1032-41.
134. Kunzmann M H, Bach N C, Bauer B, Sieber S A. a-Methylene-γ-butyrolactones attenuate Staphylococcus aureus virulence by inhibition of transcriptional regulation. Chem Sci. 2014 Feb. 4; 5(3):1158-67.
135. McAdow M, Kim H K, Dedent A C, Hendrickx APA, Schneewind O, Missiakas D M. Preventing Staphylococcus aureus sepsis through the inhibition of its agglutination in blood. PLOS Pathog. 2011 October; 7(10): e1002307.
136. Mack D, Nedelmann M, Krokotsch A, Schwarzkopf A, Heesemann J, Laufs R. Characterization of transposon mutants of biofilm-producing Staphylococcus epidermidis impaired in the accumulative phase of biofilm production: genetic identification of a hexosamine-containing polysaccharide intercellular adhesin. Infect Immun. 1994 August; 62(8):3244-53.
137. Heilmann C, Schweitzer O, Gerke C, Vanittanakom N, Mack D, Götz F. Molecular basis of intercellular adhesion in the biofilm-forming Staphylococcus epidermidis. Mol Microbiol. 1996 June; 20(5):1083-91.
138. Fluckiger U, Ulrich M, Steinhuber A, Döring G, Mack D, Landmann R, et al. Biofilm formation, icaADBC transcription, and polysaccharide intercellular adhesin synthesis by staphylococci in a device-related infection model. Infect Immun. 2005 March; 73(3):1811-9.
139. Atshan S S, Shamsudin M N, Sekawi Z, Thian Lung L T, Barantalab F, Liew Y K, et al. Comparative proteomic analysis of extracellular proteins expressed by various clonal types of Staphylococcus aureus and during planktonic growth and biofilm development. Front Microbiol. 2015; 6:524.
140. Resch A, Rosenstein R, Nerz C, Götz F. Differential gene expression profiling of Staphylococcus aureus cultivated under biofilm and planktonic conditions. Appl Environ Microbiol. 2005 May; 71(5):2663-76.
141. Duggan S, Laabei M, Alnahari A A, O'Brien E C, Lacey K A, Bacon L, et al. A Small Membrane Stabilizing Protein Critical to the Pathogenicity of Staphylococcus aureus. Infect Immun. 2020 Aug. 19; 88(9): e00162-20.
142. Bavro V, Marshall R, Symmons M. Architecture and roles of periplasmic adaptor proteins in tripartite efflux assemblies. Frontiers in Microbiology [Internet]. 2015 [cited 2023 Jan. 31]; 6. Available from: https://www.frontiersin.org/articles/10.3389/fmicb.2015.00513
143. Costa S S, Viveiros M, Amaral L, Couto I. Multidrug Efflux Pumps in Staphylococcus aureus: an Update. Open Microbiol J. 2013 Mar. 22; 7:59-71.
144. Huang J, O'Toole P W, Shen W, Amrine-Madsen H, Jiang X, Lobo N, et al. Novel Chromosomally Encoded Multidrug Efflux Transporter MdeA in Staphylococcus aureus. Antimicrobial Agents and Chemotherapy. 2004 March; 48(3):909-17.
145. Baichwal V, Liu D, Ames G F. The ATP-binding component of a prokaryotic traffic ATPase is exposed to the periplasmic (external) surface. Proceedings of the National Academy of Sciences. 1993 Jan. 15; 90(2):620-4.
146. Hung L W, Wang I X, Nikaido K, Liu P Q, Ames G F, Kim S H. Crystal structure of the ATP-binding subunit of an ABC transporter. Nature. 1998 Dec. 17; 396(6712):703-7.
147. Deibert J, Kühner D, Stahl M, Koeksoy E, Bertsche U. The Peptidoglycan Pattern of Staphylococcus carnosus TM300-Detailed Analysis and Variations Due to Genetic and Metabolic Influences. Antibiotics (Basel). 2016 Sep. 23; 5(4):33.
148. Du S, Henke W, Pichoff S, Lutkenhaus J. How FtsEX localizes to the Z ring and interacts with FtsA to regulate cell division. Molecular Microbiology. 2019; 112(3):881-95.
149. Laabei M, Uhlemann A C, Lowy F D, Austin E D, Yokoyama M, Ouadi K, et al. Evolutionary Trade-Offs Underlie the Multi-faceted Virulence of Staphylococcus aureus. PLOS Biol. 2015; 13(9): e1002229.
150. Yadav A K, Espaillat A, Cava F. Bacterial Strategies to Preserve Cell Wall Integrity Against Environmental Threats. Front Microbiol. 2018 Aug. 31; 9:2064.
151. Zouhir A, Jemli S, Omrani R, kthiri A, Jridi T, sebei K. In Silico Molecular Analysis and Docking of Potent Antimicrobial Peptides Against MurE Enzyme of Methicillin Resistant Staphylococcus Aureus. Int J Pept Res Ther. 2021 Jun. 1; 27(2):1253-63.
152. Frapwell C J, Skipp P J, Howlin R P, Angus E M, Hu Y, Coates ARM, et al. Antimicrobial Activity of the Quinoline Derivative HT61 against Staphylococcus aureus Biofilms. Antimicrob Agents Chemother. 2020 Apr. 21; 64(5): e02073-19.
153. Kouidmi I, Levesque R C, Paradis-Bleau C. The biology of Mur ligases as an antibacterial target. Molecular Microbiology. 2014; 94(2):242-53.
154. Consaul S A, Wright L F, Mahapatra S, Crick D C, Pavelka M S. An unusual mutation results in the replacement of diaminopimelate with lanthionine in the peptidoglycan of a mutant strain of Mycobacterium smegmatis. J Bacteriol. 2005 March; 187(5):1612-20.
155. Mengin-Lecreulx D, Falla T, Blanot D, van Heijenoort J, Adams D J, Chopra I. Expression of the Staphylococcus aureus UDP—N-Acetylmuramoyl-1-Alanyl-d-Glutamate: l-Lysine Ligase in Escherichia coli and Effects on Peptidoglycan Biosynthesis and Cell Growth. J Bacteriol. 1999 October; 181(19):5909-14.
156. Do T, Page J E, Walker S. Uncovering the activities, biological roles, and regulation of bacterial cell wall hydrolases and tailoring enzymes. J Biol Chem. 2020 Mar. 6; 295(10):3347-61.
157. Wang M, Buist G, van Dijl J M. Staphylococcus aureus cell wall maintenance—the multifaceted roles of peptidoglycan hydrolases in bacterial growth, fitness, and virulence. FEMS Microbiol Rev. 2022 Jun. 8; 46(5): fuac025.
158. Kong C, Neoh H min, Nathan S. Targeting Staphylococcus aureus Toxins: A Potential form of Anti-Virulence Therapy. Toxins. 2016 March; 8(3):72.
159. Abushahba M F, Mohammad H, Seleem M N. Targeting Multidrug-resistant Staphylococci with an anti-rpoA Peptide Nucleic Acid Conjugated to the HIV-1 TAT Cell Penetrating Peptide. Mol Ther Nucleic Acids. 2016 July; 5(7): e339.
160. Moy T I, Daniel A, Hardy C, Jackson A, Rehrauer O, Hwang Y S, et al. Evaluating the activity of the RNA polymerase inhibitor myxopyronin B against Staphylococcus aureus. FEMS Microbiol Lett. 2011 June; 319(2):176-9.
161. Lin Z, Wang F, Shang Z, Lin W. Biochemical and structural analyses reveal critical residues in δ subunit affecting its bindings to β′ subunit of Staphylococcus aureus RNA polymerase. Biochemical and Biophysical Research Communications. 2021 Mar. 19; 545:98-104.
162. Weiss A, Ibarra J A, Paoletti J, Carroll R K, Shaw L N. The δ Subunit of RNA Polymerase Guides Promoter Selectivity and Virulence in Staphylococcus aureus. Infect Immun. 2014 April; 82(4):1424-35.
163. Weiss A, Moore B D, Tremblay MHJ, Chaput D, Kremer A, Shaw L N. The ω Subunit Governs RNA Polymerase Stability and Transcriptional Specificity in Staphylococcus aureus. J Bacteriol. 2016 Dec. 28; 199(2): e00459-16.
164. Matsuo M, Hishinuma T, Katayama Y, Hiramatsu K. A Mutation of RNA Polymerase β′ Subunit (RpoC) Converts Heterogeneously Vancomycin-Intermediate Staphylococcus aureus (hVISA) into “Slow VISA.” Antimicrob Agents Chemother. 2015 July; 59(7):4215-25.
165. Matsuo M, Cui L, Kim J, Hiramatsu K. Comprehensive identification of mutations responsible for heterogeneous vancomycin-intermediate Staphylococcus aureus (hVISA)-to-VISA conversion in laboratory-generated VISA strains derived from hVISA clinical strain Mu3. Antimicrob Agents Chemother. 2013 Dec. 1; 57(12):5843-53.
166. Campbell E A, Korzheva N, Mustaev A, Murakami K, Nair S, Goldfarb A, et al. Structural mechanism for rifampicin inhibition of bacterial rna polymerase. Cell. 2001 Mar. 23; 104(6):901-12.
167. Zulfiana R, Suharjono null, Kuntaman null. Genetic profile mutation rpoB in clinical isolate of rifampicin-resistant Staphylococcus aureus. J Basic Clin Physiol Pharmacol. 2021 Jun. 25; 32(4):773-6.
168. Wen J D, Kuo S T, Chou HHD. The diversity of Shine-Dalgarno sequences sheds light on the evolution of translation initiation. RNA Biology. 2021 Nov. 2; 18(11):1489-500.
169. Somerville G A, Proctor R A. At the crossroads of bacterial metabolism and virulence factor synthesis in Staphylococci. Microbiol Mol Biol Rev. 2009 June; 73(2):233-48.
170. Lei M G, Cue D, Roux C M, Dunman P M, Lee C Y. Rsp Inhibits Attachment and Biofilm Formation by Repressing fnbA in Staphylococcus aureus MW2. J Bacteriol. 2011 October; 193(19):5231-41.
171. Vogwill T, MacLean R C. The genetic basis of the fitness costs of antimicrobial resistance: a meta-analysis approach. Evol Appl. 2015 March; 8(3):284-95.
172. Garcia-Gutierrez E, Walsh C J, Sayavedra L, Diaz-Calvo T, Thapa D, Ruas-Madiedo P, et al. Genotypic and Phenotypic Characterization of Fecal Staphylococcus epidermidis Isolates Suggests Plasticity to Adapt to Different Human Body Sites. Front Microbiol. 2020 Apr. 21; 11:688.
173. Altman D R, Sullivan M J, Chacko K I, Balasubramanian D, Pak T R, Sause W E, et al. Genome Plasticity of agr-Defective Staphylococcus aureus during Clinical Infection. Infect Immun. 2018 Sep. 21; 86(10): e00331-18.
174. Eren A M, Esen O C, Quince C, Vineis J H, Morrison H G, Sogin M L, Delmont T O. 2015. 'Anvi′o: an advanced analysis and visualization platform for ‘omics data. PeerJ 3: e1319.
175. Price M N, Dehal P S, Arkin A P. 2009. FastTree: computing large minimum evolution trees with profiles instead of a distance matrix. Mol Biol Evol 26:1641-1650.
176. Letunic I, Bork P. 2021. Interactive Tree Of Life (iTOL) v5: an online tool for phylogenetic tree display and annotation. Nucleic Acids Res 49: W293-W296.
177. Buchfink B, Xie C, Huson D H. 2015. Fast and sensitive protein alignment using DIAMOND. 1. Nat Methods 12:59-60.
178. Redman W K, Welch G S, Williams A C, Damron A J, Northcut W O, Rumbaugh K P. 2021. Efficacy and safety of biofilm dispersal by glycoside hydrolases in wounds. Biofilm 3:100061.
179. Fleming D, Chahin L, Rumbaugh K. 2017. Glycoside Hydrolases Degrade Polymicrobial Bacterial Biofilms in Wounds. Antimicrob Agents Chemother 61: e01998-16.
180. O'Neill E, Pozzi C, Houston P, Humphreys H, Robinson D A, Loughman A, Foster T J, O'Gara J P. 2008. A Novel Staphylococcus aureus Biofilm Phenotype Mediated by the Fibronectin-Binding Proteins, FnBPA and FnBPB. Journal of Bacteriology 190:3835-3850.
181. Turner C B, Marshall C W, Cooper V S. 2018. Parallel genetic adaptation across environments differing in mode of growth or resource availability. Evolution Letters 2:355-367.
182. Scribner M R, Santos-Lopez A, Marshall C W, Deitrick C, Cooper V S. 2020. Parallel Evolution of Tobramycin Resistance across Species and Environments, mBio 11: e00932-20.
183. Travisano M, Lenski R E. Long-Term Experimental Evolution in Escherichia coli. I V.Targets of Selection and the Specificity of Adaptation 12.

We claim:

1. An antimicrobial composition comprising a Bacillus subtilis 6D1 cell free extract (CFE) and an antibiotic.

2. The antimicrobial composition of claim 1, wherein the Bacillus subtilis 6D1 CFE is a 75% to 100% P1 glycoprotein cell-free fraction.

3. The antimicrobial composition of claim 1, wherein the composition has anti-biofilm activity in a pathogenic bacterium.

4. The antimicrobial composition of claim 1, wherein the Bacillus subtilis 6D1 CFE has anti-biofilm activity in a pathogenic bacterium.

5. The antimicrobial composition of claim 3, wherein the pathogenic bacterium is resistant to antibiotics.

6. The antimicrobial composition of claim 3, wherein anti-biofilm activity is preventing formation of biofilm, interrupting biofilm formation, inhibiting biofilm growth, or disassembling mature biofilm.

7. The antimicrobial composition of claim 1, wherein the composition inhibits growth of a pathogenic bacterium.

8. The antimicrobial composition of claim 4, wherein the Bacillus subtilis 6D1 CFE does not inhibit growth of the pathogenic bacterium.

9. The antimicrobial composition of claim 1, wherein the Bacillus subtilis 6D1 CFE inhibits bacterial quorum sensing activity in a pathogenic bacterium.

10. The antimicrobial composition of claim 9, wherein the anti-quorum sensing activity is an accessory gene regulator (Agr) quorum sensing interference mechanism.

11. The antimicrobial composition of claim 3, wherein the pathogenic bacterium is Staphylococcus aureus (S. aureus).

12. The antimicrobial composition of claim 11, wherein the S. aureus is resistant to antibiotics.

13. The antimicrobial composition of claim 11, wherein the composition reduces S. aureus fitness, virulence, and resistance to antibiotics.

14. The antimicrobial composition of claim 11, wherein the antibiotic sensitivity of S. aureus is improved.

15. The antimicrobial composition of claim 14, wherein the improved antibiotic sensitivity of S. aureus is the result of quorum sensing interference.

16. The antimicrobial composition of claim 1, wherein;
(i) the B. subtilis 6D1 CFE comprises surfactin A-D or Compound E;
(ii) the antibiotic is selected from Ampicillin, Gentamicin, and Trimethoprim, or combinations thereof; and/or
(iii) the composition further comprises a pharmaceutically acceptable carrier.

17. (canceled)

18. The antimicrobial composition of claim 2, wherein the B. subtilis 6D1 CFE 100% P1 cell-free fraction inhibits formation or disassembles S. aureus biofilm.

19. (canceled)

20. (canceled)

21. A method of obtaining the B. subtilis 6D1 CFE of claim 1 comprising:
preparing a cell-free extract from an isolated Bacillus subtilis culture; and
fractionating the cell-free extract with ethyl acetate to obtain fractionated extract with anti-biofilm activity.

22. The method of claim 21, further comprising (i) separating the fractionated extract to obtain an active B. subtilis 6D1 CFE P1 glycoprotein cell-free fraction and/or (ii) purifying the B. subtilis 6D1 CFE.

23. The method of claim 21, wherein the Bacillus subtilis 6D1 CFE P1 fraction is a 75% to 100% P1 glycoprotein cell-free fraction.

24. The method of claim 23, further comprising separating the B. subtilis 6D1 CFE 100% P1 cell-free fraction to obtain an active fraction comprising Compound E, wherein the active fraction does not contain surfactins.

25. (canceled)

26. A method of inhibiting biofilm activity in S. aureus comprising delivering or administering the composition of claim 1 to a S. aureus infection or infectious environment.

27. The method of claim 26, wherein;
(i) the composition is delivered or administered directly to the S. aureus infection or infectious environment;
(ii) the composition is delivered or administered to a biomedical or agricultural device;
(iii) the composition is administered to a subject;
(iv) the composition reduces S. aureus fitness, virulence, and resistance to antibiotics; or
(v) the biofilm activity inhibited is preventing formation of biofilm, interrupting biofilm formation, inhibiting biofilm growth, or disassembling mature biofilm.

28.-31. (canceled)
